(19) United States
(12) Patent Application Publication
(10) Pub. No.: US 2021/0369775 A1
(43) Pub. Date: Dec. 2, 2021

(54) SYSTEMS AND METHODS FOR DETERMINING THE BENEFICIAL ADMINISTRATION OF TUMOR INFILTRATING LYMPHOCYTES, AND METHODS OF USE THEREOF AND BENEFICIAL ADMINISTRATION OF TUMOR INFILTRATING LYMPHOCYTES, AND METHODS OF USE THEREOF

(71) Applicant: Iovance Biotherapeutics, Inc., San Carlos, CA (US)

(72) Inventors: Maria Fardis, San Carlos, CA (US); Heinrich Roder, Boulder, CO (US); Joanna Roder, Boulder, CO (US)

(21) Appl. No.: 16/772,135

(22) PCT Filed: Dec. 14, 2018
(86) PCT No.: PCT/US2018/065745

§ 371 Date: Jun. 11, 2020

Related U.S. Application Data
(60) Provisional application No. 62/599,385, filed on Dec. 15, 2017.

(51) Int. Cl. A61K 35/17; C12N 5/0783; A61K 31/675; A61K 38/20; G16H 20/40; G16H 20/70

(52) U.S. Cl. CPC A61K 35/17; C12N 5/0636; A61K 31/675; C12N 2501/998; G16H 20/40; G16H 20/70; C12N 2501/2302; A61K 38/2013

(57) ABSTRACT

The invention provides systems and methods for determining and predicting the effect of providing a population of tumor infiltrating lymphocytes (TILs) on a condition associated with an entity, for example the effect of providing a population of tumor infiltrating lymphocytes (TILs) on a subject having cancer. The systems and methods rely on acquiring a computer readable analytical signature from a sample of the entity, obtaining a trained model output value for the entity by inputting the computer readable analytical signature into a tier trained model panel, and classifying the entity based upon the trained model output value with a time-to-event class in an enumerated set of time-to-event classes, each of whom is associated with a different effect of providing a population of TILs to the entity.

The invention provides methods of treating cancer in a patient by administering a therapeutically effective population of TILs to the patient, which is at the same determined to be likely to benefit from the administration of TILs comparative to other cancer patients that have been administered TILs. Such methods of treatment include obtaining from the patient a tumor fragment, contacting the tumor fragment with one or more cell culture mediums, thereby performing one or more expansions of population of TILs existing in the tumor, and producing one or more subsequent populations of TILs. The invention also provides methods of treating cancer in a patient exhibiting an increased or decreased level of expression of various biological markers.

### FIELD OF THE INVENTION

The invention provides systems and methods for determining and predicting the effect of providing a population of tumor infiltrating lymphocytes (TILs) on a condition associated with an entity, for example the effect of providing a population of tumor infiltrating lymphocytes (TILs) on a subject having cancer. The systems and methods rely on acquiring a computer readable analytical signature from a sample of the entity, obtaining a trained model output value for the entity by inputting the computer readable analytical signature into a tier trained model panel, and classifying the entity based upon the trained model output value with a time-to-event class in an enumerated set of time-to-event classes, each of whom is associated with a different effect of providing a population of TILs to the entity. The invention also provides methods of treating cancer in a patient by administering a therapeutically effective population of TILs to the patient, which is at the same determined to be likely to benefit from the administration of TILs comparative to other cancer patients that have been administered TILs. Such methods of treatment include obtaining from the patient a tumor fragment, contacting the tumor fragment with one or more cell culture mediums, thereby performing one or more expansions of population of TILs existing in the tumor, and producing one or more subsequent populations of TILs. The invention also provides methods of treating cancer in a patient exhibiting an increased or decreased level of expression of various biological markers such as proteins or protein groups described herein.

### BACKGROUND OF THE INVENTION

Treatment of bulky, refractory cancers using adoptive autologous transfer of tumor infiltrating lymphocytes (TILs) represents a powerful approach to therapy for patients with poor prognoses. Gattinoni, et al., Nat. Rev. Immunol. 2006, 6, 383-393. TILs are dominated by T cells, and IL-2-based TIL expansion followed by a “rapid expansion process” (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al. J. Clin. Oncol. 2008, 26, 5233-39; Riddell, et al., Science 1992, 257, 238-41; Dudley, et al., J. Immunother. 2003, 26, 332-42. A number of approaches to improve responses to TIL therapy in melanoma and to expand TIL therapy to other tumor types have been explored with limited success, and the field remains challenging. Goff, et al., J. Clin. Oncol. 2016, 34, 2389-97; Dudley, et al., J. Clin. Oncol. 2008, 26, 5233-39; Rosenberg, et al., Clin. Cancer Res. 2011, 17, 4550-57.

### SUMMARY OF THE INVENTION

One aspect of the present disclosure provides a method of predicting whether a cancer patient is likely to benefit from administration of a population of T cells, either alone or in addition to another anti-cancer therapy, the method including the steps of: obtaining an analytical signature of a blood-derived sample from the patient, comparing the analytical signature with a training set of class-labeled analytical signatures of samples from a group of other cancer patients that have been administered T cells, and classifying the sample with a class label. In some such embodiments, the class label predicts whether the patient is likely to benefit from the administration of T cells, either alone or in addition to other anti-cancer therapies. In some such embodiments, subgroups of the other cancer patients that have been administered T cells achieved a complete response, a partial response, no response, a stable disease state, or a progressive disease state. In some embodiments, subgroups of the other cancer patients that have been administered T cells had no disease progression for about one year, about two years, about three years, about four years, about five years, or more than five years. In some embodiments, subgroups of the other cancer patients that have been administered T cells achieved progression free existence of less than 6 months, about 6 months, about 12 months, about 18 months, about 24 months, about 30 months, about 36 months, about 42 months, about 48 months, about 54 months, about 60 months, up to 60 months, or more than 60 months. For instance, in some embodiments, the class label is good, intermediate, bad, late, early, plus (+), or minus (−). In some embodiments, the class label good, late, or plus (+), is associated with progression free survival of about 24 months, about 30 months, about 36 months, about 42 months, about 48 months, about 54 months, about 60 months, up to 60 months, or more than 60 months. In some such embodiments, for example, a patient whose sample has been classified good, late, or plus (+), is likely to benefit from administration of a population of T cells. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the analytical signature is obtained by a mass spectrometry method, an electrophoresis method, or a chromatography method. In some embodiments, the analytical signature is obtained by a mass spectrometry method, and includes integrated values of selected mass spectral features over predefined m/z ranges. In some embodiments, the mass spectral features are correlated or anti-correlated with the complement system protein functional group, the acute inflammation protein functional group, the acute response protein functional group, or the acute phase protein functional group. In some embodiments, the mass spectral features are correlated or anti-correlated with the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

One aspect of the present disclosure provides a method of predicting whether a cancer patient is likely to benefit from administration of a population of tumor infiltrating lymphocytes (TILs), either alone or in addition to another anti-cancer therapy, the method including the steps of: obtaining an analytical signature of a blood-derived sample from the patient, comparing the analytical signature with a training set of class-labeled analytical signatures of samples from a group of other cancer patients that have been administered TILs, and classifying the sample with a class label. In some such embodiments, the class label predicts whether the patient is likely to benefit from the administration of TILs, either alone or in addition to other anti-cancer therapies. In some such embodiments, subgroups of the other cancer patients that have been administered TILs achieved a complete response, a partial response, no response, a stable disease state, or a progressive disease state. In some embodiments, subgroups of the other cancer patients that have been administered TILs had no disease progression for about one year, about two years, about three years, about four years, about five years, or more than five years. In some embodiments, subgroups of the other cancer patients that have been administered TILs achieved progression free existence of less than 6 months, about 6 months, about 12 months, about 18 months, about 24 months, about 30 months, about 36 months, about 42 months, about 48 months, about 54 months, about 60 months, up to 60 months, or more than 60 months. For instance, in some embodiments, the class label is good, intermediate, bad, late, early, plus (+), or minus (−). In some embodiments, the class label good, late, or plus (+), is associated with progression free survival of about 24 months, about 30 months, about 36 months, about 42 months, about 48 months, about 54 months, about 60 months, up to 60 months, or more than 60 months. In some such embodiments, for example, a patient whose sample has been classified good, late, or plus (+), is likely to benefit from administration of a population of TILs. In some embodiments, the analytical signature is obtained by a mass spectrometry method, an electrophoresis method, or a chromatography method. In some embodiments, the analytical signature is obtained by a mass spectrometry method, and includes integrated values of selected mass spectral features over predefined m/z ranges. In some embodiments, the mass spectral features are correlated or anti-correlated with the complement system protein functional group, the acute inflammation protein functional group, the acute response protein functional group, or the acute phase protein functional group. In some embodiments, the mass spectral features are correlated or anti-correlated with the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In one embodiment, the invention relates to a system for screening a target entity to determine whether it has a first property, the system including at least one processor and memory addressable by the at least one processor, the memory storing at least one program for execution by the at least one processor, the at least one program including instructions for: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property includes a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In one embodiment, the acquiring includes acquiring values of selected m/z of the sample using a spectrometer. In one embodiment, the acquiring includes acquiring integrated values of selected m/z of the sample across each subset in a plurality of predetermined subsets of m/z ranges using a spectrometer thereby forming the first computer readable analytical signature. In one embodiment, each subset in the plurality of predetermined subsets of m/z ranges is selected from Table 16. In one embodiment, the spectrometer is a mass-spectrometer conducted in positive ion mode.

In one embodiment, the invention relates to a system for screening a target entity to determine whether it has a first property, the system including at least one processor and memory addressable by the at least one processor, the memory storing at least one program for execution by the at least one processor, the at least one program including instructions for: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property includes a discernable effect of providing a population of tumor infiltrating lymphocytes (TILs) on a condition associated with the first entity. In one embodiment, the acquiring includes acquiring values of selected m/z of the sample using a spectrometer. In one embodiment, the acquiring includes acquiring integrated values of selected m/z of the sample across each subset in a plurality of predetermined subsets of m/z ranges using a spectrometer thereby forming the first computer readable analytical signature. In one embodiment, each subset in the plurality of predetermined subsets of m/z ranges is selected from Table 16. In one embodiment, the spectrometer is a mass-spectrometer conducted in positive ion mode.

In some embodiments, the acquiring A) includes acquiring integrated m/z values of the sample across each respective subset in a plurality of predetermined subsets of m/z ranges using a spectrometer thereby forming the first computer readable analytical signature, the first tier trained model panel includes a plurality of first master-classifiers; and the inputting the first computer readable analytical signature of the entity into the first tier trained model panel includes: (i) providing each respective first master-classifier in the plurality of first master-classifiers with the first computer readable analytical signature thereby obtaining a corresponding first component output value of the respective first master-classifier in a plurality of first component output values, and (ii) combining the plurality of first component output values to form the first trained model output value for the entity.

In some embodiments, the at least one program further includes instructions for: applying a cutoff threshold to each first component output value in the plurality of first component output values prior to the combining (ii), and the combining the plurality of first component output values to form the first trained model output value for the target entity (ii) includes an unweighted voting across the plurality of first component output values to form the first trained model output value for the target entity.

In one embodiment, a respective first master-classifier in the plurality of first master-classifiers includes a logistic expression of a plurality of mini-classifiers, and each respective mini-classifier in the plurality of mini-classifiers contributes to the logistic expression using a unique subset of the plurality of predetermined subsets of m/z ranges that corresponds to the respective mini-classifier. In one embodiment, each respective mini-classifier in the plurality of mini-classifiers contributes to the logistic expression by applying the unique subset of the plurality of predetermined subsets of m/z ranges that corresponds to the respective mini-classifier against a different test set associated with the first master-classifier using nearest neighbor analysis, and the different test set includes a first plurality of test entities, and for each respective test entity in the first plurality of test entities, (i) measured values across each m/z subset in the plurality of predetermined subsets of m/z ranges from a test sample from the respective test entity and (ii) a specified time-to-event class in the enumerated set of time-to-event classes for the respective test entity. In one embodiment, the nearest neighbor analysis is k-nearest neighbor analysis, wherein k is a positive integer. In one embodiment, each respective first master-classifier in the plurality of first master-classifiers includes a different logistic expression of a different plurality of mini-classifiers, and each respective mini-classifier in the different plurality of mini-classifiers for a respective first master-classifier in the plurality of first master-classifiers contributes to the corresponding logistic expression by applying a unique subset of the plurality of predetermined subsets of m/z ranges that corresponds to the respective mini-classifier against a different test set, in a plurality of test sets, wherein the different test set is associated with the respective first master-classifier, using nearest neighbor analysis, and the different test set associated with the respective first master-classifier includes a respective plurality of test entities, and for each respective test entity in the respective plurality of test entities, (i) measured integrated m/z values of a test sample from a respective test entity in the respectively plurality of test entities across each respective subset in the plurality of predetermined subsets of m/z ranges and (ii) a specified time-to-event class in the enumerated set of time-to-event classes. In one embodiment, there is partial overlap between each respective test set in the plurality of test sets.

In one embodiment, each predetermined subset of m/z ranges in the plurality of predetermined subsets of m/z ranges is centered on an m/z value provided in column one of Table 21. In one embodiment, at least 10 predetermined subsets of m/z ranges in the plurality of predetermined subsets of m/z ranges is centered on a different m/z value provided in column one of Table 21. In one embodiment, at least 40 predetermined subsets of m/z ranges in the plurality of predetermined subsets of m/z ranges is centered on a different m/z value provided in column one of Table 21. In one embodiment, at least 80 predetermined subsets of m/z ranges in the plurality of predetermined subsets of m/z ranges is centered on a different m/z value provided in column one of Table 21. In one embodiment, at least 120 predetermined subsets of m/z ranges in the plurality of predetermined subsets of m/z ranges is centered on a different m/z value provided in column one of Table 21.

In some embodiments, the acquiring A) includes: acquiring integrated m/z values of the sample across each respective subset in a first plurality of predetermined subsets of m/z ranges thereby forming the first computer readable analytical signature, and acquiring integrated m/z values of the sample across each respective subset in a second plurality of predetermined subsets of m/z ranges thereby forming a second computer readable analytical signature, and the classifying C) includes: classifying the target entity with a first time-to-event class in the enumerated set of time-to-event classes when the first trained model output value is in a first value range; and performing a follow up procedure when the first trained model output value is in a second value range; wherein the follow up procedure includes: i) inputting the second computer readable analytical signature of the target entity into a second tier trained model panel thereby obtaining a second trained model output value for the entity; and ii) classifying the target entity based upon the second trained model output value with a time-to-event class in the enumerated set of time-to-event classes. In one embodiment, the first tier trained model panel includes a plurality of first master-classifiers; and the inputting the first computer readable analytical signature of the target entity into the first tier trained model panel includes: (i) providing each respective first master-classifier in the plurality of first master-classifiers with the first computer readable analytical signature thereby obtaining a corresponding first component output value of the respective first master-classifier in a plurality of first component output values, and (ii) combining the plurality of first component output values to form the first trained model output value for the entity. In one embodiment, the second tier trained model panel includes a plurality of second master-classifiers; and the inputting the second computer readable analytical signature of the target entity into the second tier trained model panel includes: (i) providing each respective second master-classifier in the plurality of second master-classifiers with the second computer readable analytical signature thereby obtaining a corresponding second component output value of the respective second master-classifier in a plurality of second component output values, and (ii) combining the plurality of second component output values to form the second trained model output value for the entity. In one embodiment, the at least one program further includes instructions for: applying a cutoff threshold to each second component output value in the plurality of second component output values prior to the combining the plurality of second component output values (ii), and the combining the plurality of second component output values to form the second trained model output value for the entity (ii) includes an unweighted voting across the plurality of second component output values to form the second trained model output value for the entity. In one embodiment, a respective first master-classifier in the plurality of first master-classifiers includes a first logistic expression of the first plurality of mini-classifiers, each respective mini-classifier in the first plurality of mini-classifiers contributes to the first logistic expression using a unique subset of the plurality of predetermined subsets of m/z ranges that corresponds to the respective mini-classifier, a respective second master-classifier in the plurality of second master-classifiers includes a second logistic expression of the second plurality of mini-classifiers, and each respective mini-classifier in the second plurality of mini-classifiers contributes to the second logistic expression using a unique subset of the plurality of predetermined subsets of m/z ranges that corresponds to the respective mini-classifier. In one embodiment, each respective mini-classifier in the first plurality of mini-classifiers contributes to the first logistic expression by applying the unique subset of the plurality of predetermined subsets of m/z ranges that corresponds to the respective mini-classifier against a different test set associated with the first master-classifier using nearest neighbor analysis, the different test set includes a first plurality of test entities, and for each respective test entity in the first plurality of test entities, (i) measured values for the selected m/z of a test sample from the respective test entity at each respective subset in the plurality of predetermined subsets of m/z ranges and (ii) a specified time-to-event class in the enumerated set of time-to-event classes, each respective mini-classifier in the second plurality of mini-classifiers contributes to the second logistic expression by applying the unique subset of the plurality of predetermined subsets of m/z ranges that corresponds to the respective mini-classifier against a different test set associated with the second master-classifier using nearest neighbor analysis, the different test set includes a second plurality of test entities, and for each respective test entity in the second plurality of test entities, (i) measured values for the selected m/z of a test sample from the respective test entity at each respective subset in the plurality of predetermined subsets of m/z ranges and (ii) a specified time-to-event class in the enumerated set of time-to-event classes. In one embodiment, the nearest neighbor analysis is k-nearest neighbor analysis, wherein k is a positive integer.

In some embodiments, each respective first master-classifier in the plurality of first master-classifiers includes a different logistic expression of a different plurality of mini-classifiers, and each respective mini-classifier in the different plurality of mini-classifiers for a respective first master-classifier in the plurality of first master-classifiers contributes to the first logistic expression by applying a unique subset of the plurality of predetermined subsets of m/z ranges that corresponds to the respective mini-classifier against a different test set, in a first plurality of test sets, wherein the different test set is associated with the respective first master-classifier using nearest neighbor analysis, the different test set associated with the respective first master-classifier includes a respective plurality of test entities, and for each respective test entity in the plurality of test entities, (i) measured values for the selected m/z of a test sample from a respective test entity in the respectively plurality of test entities at each respective subset in the plurality of predetermined subsets of m/z ranges and (ii) a specified time-to-event class in the enumerated set of time-to-event classes, each respective second master-classifier in the plurality of second master-classifiers includes a different logistic expression of a different plurality of mini-classifiers, and each respective mini-classifier in the different plurality of mini-classifiers for a respective second master-classifier in the plurality of second master-classifiers contributes to the second logistic expression by applying a unique subset of the plurality of predetermined subsets of m/z ranges that corresponds to the respective mini-classifier against a different test set, in a second plurality of test sets, wherein the different test set is associated with the respective second master-classifier, using nearest neighbor analysis, the different test set associated with the respective second master-classifier includes a respective plurality of test entities, and for each respective test entity in the respective plurality of test entities, (i) measured values for the selected m/z of a test sample from a respective test entity in the respectively plurality of test entities at each respective subset in the plurality of predetermined subsets of m/z ranges and (ii) a specified time-to-event class in the enumerated set of time-to-event classes.

In some embodiments, each predetermined subset of m/z ranges in the first plurality of predetermined subsets of m/z ranges is centered on an m/z value provided in column one of Table 21, and each predetermined subset of m/z ranges in the second plurality of predetermined subsets of m/z ranges is centered on an m/z value provided in column two of Table 21. In some embodiments, at least 10 predetermined subsets of m/z ranges in the first plurality of predetermined subsets of m/z ranges is centered on a different m/z value provided in column one of Table 21, and at least 4 predetermined subsets of m/z ranges in the second plurality of predetermined subsets of m/z ranges is centered on a different m/z value provided in column two of Table 21. In some embodiments, at least 40 predetermined subsets of m/z ranges in the first plurality of predetermined subsets of m/z ranges is centered on a different m/z value provided in column one of Table 21, and at least 8 predetermined subsets of m/z ranges in the second plurality of predetermined subsets of m/z ranges is centered on a different m/z value provided in column two of Table 21. In some embodiments, at least 80 predetermined subsets of m/z ranges in the first plurality of predetermined subsets of m/z ranges is centered on a different m/z value provided in column one of Table 21, and at least 12 predetermined subsets of m/z ranges in the second plurality of predetermined subsets of m/z ranges is centered on a different m/z value provided in column two of Table 21. In some embodiments, at least 120 predetermined subsets of m/z ranges in the plurality of predetermined subsets of m/z ranges is centered on a different m/z value provided in column one of Table 21, and at least 16 predetermined subsets of m/z ranges in the second plurality of predetermined subsets of m/z ranges is centered on a different m/z value provided in column two of Table 21.

In some embodiments, the acquiring A) includes deriving characteristic values of the sample by electrophoresis or chromatography. In some embodiments, the enumerated set of classes consists of good, intermediate, bad, late, early, plus (+), and minus (−). In some embodiments, the enumerated set of classes includes good, intermediate, bad, late, early, plus (+), and minus (−). In some embodiments, the discernable effect for the good, late, or plus (+) class is progression free existence of the entity for a first epic commencing at the first time point, and the first epic is selected from the group consisting of about 24 months, about 30 months, about 36 months, about 42 months, about 48 months, about 54 months, about 60 months, up to 60 months, and more than 60 months. In some embodiments, the discernable effect for the good, late or plus (+) class occurs with a likelihood that is greater than a predetermined threshold level. In some embodiments, the predetermined threshold level is fifty percent, sixty percent, seventy percent, eighty percent, or ninety percent. In some embodiments, the providing the population of T cells further includes co-providing another therapy with the population of T cells for the condition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the providing the population of TILs further includes co-providing another therapy with the population of TILs for the condition.

In some embodiments, the at least one program further includes instructions for: training, prior to the inputting B), one or more models to thereby form the first tier trained model. In one embodiment, the training includes: obtaining a training set that represents a plurality of training entities, wherein each training entity in the plurality of training entities has the condition and, for each respective training entity, the training set includes (i) a computer readable analytical signature from a sample of the respective training entity and (ii) an effect that providing the population of TILs had on the condition, and using the training set to train the one or more models thereby forming the first tier trained model panel. In one embodiment, the enumerated set of classes consists of good, intermediate, bad, late, early, plus (+), and minus (−), and the training set includes a different plurality of training entities for each class in the enumerated set of classes. In one embodiment, the enumerated set of classes includes good, intermediate, bad, late, early, plus (+), and minus (−), and the training set includes a different plurality of training entities for each class in the enumerated set of classes.

In some embodiments, the training set includes: a first subset of entities that have been provided T cells and had no condition progression for a first period of time, a second subset of entities that have been provided T cells and had no condition progression for a second period of time, and a third subset of entities that have been provided T cells and had no condition progression for a third period of time. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In one embodiment, the first period of time, the second period time and third period of time are each independently selected from the group consisting of about one year, about two years, about three years, about four years, about five years, and more than five years. In one embodiment, the first period of time, the second period time and third period of time are each independently selected from the group consisting of less than 6 months, about 6 months, about 12 months, about 18 months, about 24 months, about 30 months, about 36 months, about 42 months, about 48 months, about 54 months, about 60 months, up to 60 months, and more than 60 months.

In some embodiments, the training set includes: a first subset of entities that have been provided TILs and had no condition progression for a first period of time, a second subset of entities that have been provided TILs and had no condition progression for a second period of time, and a third subset of entities that have been provided TILs and had no condition progression for a third period of time. In one embodiment, the first period of time, the second period time and third period of time are each independently selected from the group consisting of about one year, about two years, about three years, about four years, about five years, and more than five years. In one embodiment, the first period of time, the second period time and third period of time are each independently selected from the group consisting of less than 6 months, about 6 months, about 12 months, about 18 months, about 24 months, about 30 months, about 36 months, about 42 months, about 48 months, about 54 months, about 60 months, up to 60 months, and more than 60 months.

In some embodiments, the target entity is human and the sample of the entity is a serum sample or a plasma sample from the entity. In some embodiments, each subset in the first plurality of predetermined subsets of m/z ranges is correlated or anti-correlated with the complement system protein functional group, the acute inflammation protein functional group, the acute response protein functional group, or the acute phase protein functional group. In some embodiments, each subset in the first plurality of predetermined subsets of m/z ranges is correlated or anti-correlated with a level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the condition is cancer. In some embodiments, the condition is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the condition is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁻/HER2⁺), triple negative breast cancer (ER⁻/PR⁻/HER2⁻), double-refractory melanoma, and uveal (ocular) melanoma. In some embodiments, the first tier trained model panel consists of a single support vector machine. In some embodiments, the first tier trained model panel consists of a plurality of support vector machines.

In some embodiments, the invention relates to a method for screening a target entity to determine whether it has a first property, method including: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property includes a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the invention relates to a method for screening a target entity to determine whether it has a first property, method including: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property includes a discernable effect of providing a population of tumor infiltrating lymphocytes (TILs) on a condition associated with the first entity.

In one embodiment, the invention provides a method of predicting whether a cancer patient is likely to benefit from administration of a population of T cells, either alone or in addition to another anti-cancer therapy, including the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of predicting whether a cancer patient is likely to benefit from administration of a population of tumor infiltrating lymphocytes (TILs), either alone or in addition to another anti-cancer therapy, including the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In some embodiments, the analytical signature is obtained by a mass spectrometry method, an electrophoresis method, or a chromatography method. In some embodiments, the analytical signature is obtained by a mass spectrometry method, and includes integrated intensity values of selected mass spectral features over predefined m/z ranges. In some embodiments, the mass spectral m/z ranges are one or more ranges listed in Table 16. In some embodiments, the mass spectral features are one or more features listed in Table 22. In some embodiments, mass-spectrometry is conducted in positive ion mode.

In one embodiment, the invention relates to a method of treating cancer in a patient having a cancer-related tumor, wherein the patient is likely to benefit from administration of T cells comparative to a group of other cancer patients that have been administered T cells, including the steps of: obtaining from the patient a first population of T cells; contacting the population with a first cell culture medium; and performing an initial expansion of the first population of T cells in the first cell culture medium to obtain a second population of T cells. In some embodiments, the second population of T cells is at least 5-fold greater in number than the first population of T cells. In some embodiments, the first cell culture medium includes IL-2. In some embodiments, the method further includes performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium includes IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further includes harvesting the third population of T cells. In some embodiments, the method further includes administering a therapeutically effective portion of the third population of T cells to the patient. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention relates to a method of treating cancer in a patient having a cancer-related tumor, wherein the patient is likely to benefit from administration of TILs comparative to a group of other cancer patients that have been administered TILs, including the steps of: obtaining from the patient a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium includes IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium includes IL-2, OKT-3 (anti-CD3 antibody), and irradiated allogeneic peripheral blood mononuclear cells (PBMCs); and wherein the rapid expansion is performed over a period of 14 days or less; harvesting the third population of TILs; and administering a therapeutically effective portion of the third population of TILs to the patient.

In some embodiments, the likelihood of beneficial administration of T cells is determined by a serum based analytical assay including: obtaining an analytical signature of a blood-derived sample from the patient; comparing the analytical signature with a training set of analytical signatures of samples from a group of other cancer patients that have been administered T cells, wherein the analytical signatures are class-labeled good, intermediate, bad, late, early, plus (+), or minus (−); and classifying the patient sample with the class label good, late, or plus (+). In some embodiments, subgroups of the other cancer patients that have been administered T cells achieved a complete response, a partial response, no response, a stable disease state, or a progressive disease state. In some embodiments, subgroups of the other cancer patients that have been administered T cells had no disease progression for about one year, about two years, about three years, about four years, about five years, or more than five years. In some embodiments, subgroups of the other cancer patients that have been administered T cells achieved progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the class label good, late, or plus (+), is associated with progression free survival of about 24 months, about 30 months, about 36 months, about 42 months, about 48 months, about 54 months, about 60 months, up to 60 months, or more than 60 months. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the likelihood of beneficial administration of TILs is determined by a serum based analytical assay including: obtaining an analytical signature of a blood-derived sample from the patient; comparing the analytical signature with a training set of analytical signatures of samples from a group of other cancer patients that have been administered TILs, wherein the analytical signatures are class-labeled good, intermediate, bad, late, early, plus (+), or minus (−); and classifying the patient sample with the class label good, late, or plus (+). In some embodiments, subgroups of the other cancer patients that have been administered TILs achieved a complete response, a partial response, no response, a stable disease state, or a progressive disease state. In some embodiments, subgroups of the other cancer patients that have been administered TILs had no disease progression for about one year, about two years, about three years, about four years, about five years, or more than five years. In some embodiments, subgroups of the other cancer patients that have been administered TILs achieved progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the class label good, late, or plus (+), is associated with progression free survival of about 24 months, about 30 months, about 36 months, about 42 months, about 48 months, about 54 months, about 60 months, up to 60 months, or more than 60 months.

In some embodiments, the analytical signature is obtained by a mass spectrometry method, an electrophoresis method, or a chromatography method. In some embodiments, the analytical signature is obtained by a mass spectrometry method, and the analytical signature includes integrated intensity values of selected mass spectral features over predefined m/z ranges. In some embodiments, the mass spectral features are correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In some embodiments, the invention relates to a method of treating cancer in a patient having a cancer-related tumor, wherein the patient is likely to benefit from administration of T cells, including the steps of: obtaining a first population of T cells; contacting the population with a first cell culture medium; and performing an initial expansion of the first population of T cells in the first cell culture medium to obtain a second population of T cells. In some embodiments, the second population of T cells is at least 5-fold greater in number than the first population of T cells. In some embodiments, the first cell culture medium includes IL-2. In some embodiments, the method further includes performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium includes IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further includes harvesting the third population of T cells. In some embodiments, the method further includes administering a therapeutically effective portion of the third population of T cells to the patient. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the invention relates to a method of treating cancer in a patient having a cancer-related tumor, wherein the patient is likely to benefit from administration of TILs, including the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium includes IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium includes IL-2, OKT-3 (anti-CD3 antibody), and irradiated allogeneic peripheral blood mononuclear cells (PBMCs); and wherein the rapid expansion is performed over a period of 14 days or less; harvesting the third population of TILs; and administering a therapeutically effective portion of the third population of TILs to the patient.

In some embodiments, the likelihood of beneficial administration of T cells is determined by a serum based analytical method, including the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the likelihood of beneficial administration of TILs is determined by a serum based analytical method, including the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In some embodiments, the analytical signature is obtained by a mass spectrometry method, an electrophoresis method, or a chromatography method. In some embodiments, the analytical signature is obtained by a mass spectrometry method, and the analytical signature includes integrated intensity values of selected mass spectral features over predefined m/z ranges. In some embodiments, the mass spectral m/z ranges are one or more ranges listed in Table 16. In some embodiments, the mass spectral features are one or more features listed in Table 22. In some embodiments, mass-spectrometry is conducted in positive ion mode. In some embodiments, the initial expansion is performed over a period of 21 days or less. In some embodiments, the initial expansion is performed over a period of 11 days or less. In some embodiments, the rapid expansion is performed over a period of 7 days or less. In some embodiments, the IL-2 is present at an initial concentration of between 1000 IU/mL and 6000 IU/mL in the first cell culture medium. In some embodiments, the IL-2 is present at an initial concentration of between 1000 IU/mL and 6000 IU/mL and the OKT-3 antibody is present at an initial concentration of about 30 ng/mL in the second cell culture medium. In some embodiments, the initial expansion is performed using a gas permeable container. In some embodiments, the rapid expansion is performed using a gas permeable container. In some embodiments, the first cell culture medium further includes a cytokine selected from the group consisting of IL-4, IL-7, IL-15, IL-21, and combinations thereof. In some embodiments, the second cell culture medium further includes a cytokine selected from the group consisting of IL-4, IL-7, IL-15, IL-21, and combinations thereof.

In some embodiments, the method further includes the step of treating the patient with a non-myeloablative lymphodepletion regimen prior to administering the third population of T cells to the patient. In some embodiments, the non-myeloablative lymphodepletion regimen includes the steps of administration of cyclophosphamide at a dose of 60 mg/m²/day for two days followed by administration of fludarabine at a dose of 25 mg/m²/day for five days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the method further includes the step of treating the patient with a non-myeloablative lymphodepletion regimen prior to administering the third population of TILs to the patient. In some embodiments, the non-myeloablative lymphodepletion regimen includes the steps of administration of cyclophosphamide at a dose of 60 mg/m²/day for two days followed by administration of fludarabine at a dose of 25 mg/m²/day for five days.

In some embodiments, the method further includes the step of treating the patient with a high-dose IL-2 regimen starting on the day after administration of the third population of T cells to the patient. In some embodiments, the high-dose IL-2 regimen further includes aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the method further includes the step of treating the patient with a high-dose IL-2 regimen starting on the day after administration of the third population of TILs to the patient. In some embodiments, the high-dose IL-2 regimen further includes aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance.

In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁻/PR⁺/HER2⁺), triple negative breast cancer (ER⁻/PR⁻/HER2⁺), double-refractory melanoma, and uveal (ocular) melanoma.

In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method including the steps of: obtaining a first population of T cells; and contacting the population with a first cell culture medium. In some embodiments, the method further includes performing an initial expansion of the first population of T cells in the first cell culture medium to obtain a second population of T cells. In some embodiments, the second population of T cells is at least 5-fold greater in number than the first population of T cells. In some embodiments, the first cell culture medium includes IL-2. In some embodiments, the method further includes performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium includes IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further includes harvesting the third population of T cells. In some embodiments, the method further includes administering a therapeutically effective portion of the third population of T cells to the patient. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the level of protein expression is increased or decreased as compared to a healthy subject. In some embodiments, the level of protein expression is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method including the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium includes IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium includes IL-2, OKT-3 (anti-CD3 antibody), and irradiated allogeneic peripheral blood mononuclear cells (PBMCs); and wherein the rapid expansion is performed over a period of 14 days or less; harvesting the third population of TILs; and administering a therapeutically effective portion of the third population of TILs to the patient. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁻/HER2⁺), triple negative breast cancer (ER⁻/PR⁻/HER2⁺), double-refractory melanoma, and uveal (ocular) melanoma. In some embodiments, the level of protein expression is increased or decreased as compared to a healthy subject. In some embodiments, the level of protein expression is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the invention relates to a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method including the steps of: obtaining a first population of T cells; and contacting the population with a first cell culture medium. In some embodiments, the method further includes performing an initial expansion of the first population of T cells in the first cell culture medium to obtain a second population of T cells. In some embodiments, the second population of T cells is at least 5-fold greater in number than the first population of T cells. In some embodiments, the first cell culture medium includes IL-2. In some embodiments, the method further includes performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium includes IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further includes harvesting the third population of T cells. In some embodiments, the method further includes administering a therapeutically effective portion of the third population of T cells to the patient. In some embodiments, the different cancer patient has been previously treated with a population of T cells. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the other cancer patient achieved a post-treatment complete response, partial response, or a stable disease state. In some embodiments, the other cancer patient achieved had no post-treatment disease progression for about one year, about two years, about three years, about four years, about five years, or more than five years. In some embodiments, the other cancer patient achieved post-treatment progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the level of protein expression similarity is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the invention relates to a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method including the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium includes IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium includes IL-2, OKT-3 (anti-CD3 antibody), and irradiated allogeneic peripheral blood mononuclear cells (PBMCs); and wherein the rapid expansion is performed over a period of 14 days or less; harvesting the third population of TILs; and administering a therapeutically effective portion of the third population of TILs to the patient, wherein the different cancer patient has been previously treated with a population of TILs. In some embodiments, the other cancer patient achieved a post-treatment complete response, partial response, or a stable disease state. In some embodiments, the other cancer patient achieved had no post-treatment disease progression for about one year, about two years, about three years, about four years, about five years, or more than five years. In some embodiments, the other cancer patient achieved post-treatment progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the level of protein expression similarity is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the initial expansion is performed over a period of 21 days or less. In some embodiments, the initial expansion is performed over a period of 11 days or less. In some embodiments, the rapid expansion is performed over a period of 7 days or less. In some embodiments, the IL-2 is present at an initial concentration of between 1000 IU/mL and 6000 IU/mL in the first cell culture medium. In some embodiments, the IL-2 is present at an initial concentration of between 1000 IU/mL and 6000 IU/mL and the OKT-3 antibody is present at an initial concentration of about 30 ng/mL in the second cell culture medium. In some embodiments, the initial expansion is performed using a gas permeable container. In some embodiments, the rapid expansion is performed using a gas permeable container. In some embodiments, the first cell culture medium further includes a cytokine selected from the group consisting of IL-4, IL-7, IL-15, IL-21, and combinations thereof. In some embodiments, the second cell culture medium further includes a cytokine selected from the group consisting of IL-4, IL-7, IL-15, IL-21, and combinations thereof.

In some embodiments, the method further includes the step of treating the patient with a non-myeloablative lymphodepletion regimen prior to administering the third population of T cells to the patient. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the non-myeloablative lymphodepletion regimen includes the steps of administration of cyclophosphamide at a dose of 60 mg/m²/day for two days followed by administration of fludarabine at a dose of 25 mg/m²/day for five days.

In some embodiments, the method further includes the step of treating the patient with a non-myeloablative lymphodepletion regimen prior to administering the third population of TILs to the patient. In some embodiments, the non-myeloablative lymphodepletion regimen includes the steps of administration of cyclophosphamide at a dose of 60 mg/m²/day for two days followed by administration of fludarabine at a dose of 25 mg/m²/day for five days.

In some embodiments, the method further includes the step of treating the patient with a high-dose IL-2 regimen starting on the day after administration of the third population of T cells to the patient. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the high-dose IL-2 regimen further includes aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance.

In some embodiments, the method further includes the step of treating the patient with a high-dose IL-2 regimen starting on the day after administration of the third population of TILs to the patient. In some embodiments, the high-dose IL-2 regimen further includes aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance.

### BRIEF DESCRIPTION OF THE DRAWINGS

The foregoing summary, as well as the following detailed description of the invention, will be better understood when read in conjunction with the appended drawings.

FIG. 1 illustrates the Kaplan-Meier plot of progression-free survival (PFS) for the analysis cohort.

FIG. 2 illustrates the Kaplan-Meier plot of PFS by BDX008 classification for the analysis cohort.

FIG. 3 illustrates the Kaplan-Meier plot of PFS by IL2 test classification for the analysis cohort of 85 patients.

FIG. 4 illustrates the distribution of bin normalization scalars by response group.

FIG. 5 illustrates an example of features defined in the dataset.

FIGS. 6A, 6B, and 6C illustrate the batch correction plots pre-correction.

FIGS. 7A, 7B, and 7C illustrate the batch correction plots post-correction.

FIG. 8 illustrates the distribution of PIC normalization scalars by response group.

FIG. 9 illustrates the diagnostic cortex.

FIG. 10 illustrates the Gene (Protein) Set Enrichment Analysis approach to associating mass spectral features and test classifications with biological functions.

FIG. 11 illustrates the schema of Classifier 1.

FIG. 12 illustrates the Kaplan-Meier plot of PFS by Classifier 1 classifications.

FIG. 13 illustrates the classification schema for Classifier 2.

FIG. 14 illustrates the Kaplan-Meier plot of PFS by Classifier 2 classifications.

FIG. 15 illustrates the running sum, RS, as a function of protein index, i, for FIG. 15(a): acute inflammation, FIG. 15(b): complement, FIG. 15(c): acute response, and FIG. 15(d): acute phase.

FIG. 16 illustrates a TIL expansion and treatment process. Step 1 refers to the addition of 4 tumor fragments into 10 G-Rex 10 flasks. At step 2, approximately 40×10⁶ TILs or greater are obtained. At step 3, a split occurs into 36 G-Rex 100 flasks for REP. TILs are harvested by centrifugation at step 4. Fresh TIL product is obtained at step 5 after a total process time of approximate 43 days, at which point TILs may be infused into a patient.

FIG. 17 illustrates a treatment protocol for use with TILs. Surgery and tumor resection occurs at the start, and lymphodepletion chemo refers to non-myeloablative lymphodepletion with chemotherapy as described elsewhere herein.

FIG. 18 illustrates an exemplary system topology for a discovery system for screening a target entity to determine whether it has a first property, in accordance with an embodiment of the present disclosure.

FIG. 19 illustrates a discovery system for screening a target entity to determine whether it has a first property, in accordance with an embodiment of the present disclosure.

FIG. 20 illustrates exemplary data structures, in accordance with an embodiment of the present disclosure.

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is the amino acid sequence of a recombinant human IL-4 protein.

SEQ ID NO:6 is the amino acid sequence of a recombinant human IL-7 protein.

SEQ ID NO:7 is the amino acid sequence of a recombinant human IL-15 protein.

SEQ ID NO:8 is the amino acid sequence of a recombinant human IL-21 protein.

### DETAILED DESCRIPTION OF THE INVENTION

The invention relates to determining the beneficial administration of T cells, for example tumor infiltrating lymphocytes (TILs), to a cancer patient, including systems and methods of determining such beneficial administration, and methods of treatment including administration of TILs to cancer patients likely to benefit from such administration. The methods include the use of the mass spectrum of the cancer patient's serum or plasma sample acquired pre-treatment, and a general purpose computer configured as a classifier which assigns a class label to the mass spectrum. The class label can take the form of “late,” or an equivalent label, e.g., “good,” or “early,” or an equivalent label, e.g., “bad,” with the class label “late” or “good” indicating that the patient is a member of a class of patients that are likely to obtain relatively greater benefit from TILs therapy compared to patients that are members of the class of patients having the class label “early” or “bad.” The particular moniker used for the class label is not particularly important. Predictive tests for a melanoma patient benefit from an antibody drug and related classifier development methods are described for example in International Patent Application Publication WO 2017/011439, the content of which is incorporated herein in its entirety. Progression-free survival, and/or overall survival, are indicators for assessing the benefit of TILs therapy. Hence, when considering the meaning of the labels late and early, or good and bad, the “relatively greater benefit” associated with the late or good label means a patient whose sample is assigned the late or good label is likely to have significantly greater, i.e., longer progression-free and/or overall survival than a patient with the early or bad class label.

### Definitions

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a mammalian subject's body.

The term “ex vivo” refers to an event that takes place outside of a mammalian subject's body, in an artificial environment.

The term “in vitro” refers to an event that takes places in a test system. In vitro assays encompass cell-based assays in which alive or dead cells may be are employed and may also encompass a cell-free assay in which no intact cells are employed.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. Preferably, the peripheral blood mononuclear cells are irradiated allogeneic peripheral blood mononuclear cells.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab.

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| Muromonab heavy | NQKFKDKATL TTDKSSSTAY MQLSSLTSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| Muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4902,502, the disclosures of which are incorporated by reference herein. Formulations of IL-2 suitable for use in the invention are described in U.S. Pat. No. 6,706,289, the disclosure of which is incorporated by reference herein.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | MHKCDITLQE IIKTLNSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 6 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 7 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 8 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, N.J., USA (Cat. No. CYT-211) and ThermoFisher Scientific, Inc., Waltham, Mass., USA (human IL-4 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, N.J., USA (Cat. No. CYT-254) and ThermoFisher Scientific, Inc., Waltham, Mass., USA (human IL-7 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15, and includes all forms of IL-15 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, N.J., USA (Cat. No. CYT-230-b) and ThermoFisher Scientific, Inc., Waltham, Mass., USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, N.J., USA (Cat. No. CYT-408-b) and ThermoFisher Scientific, Inc., Waltham, Mass., USA (human IL-21 recombinant protein, Cat. No. 14-8219-80). The amino acid sequence of recombinant human IL-21 suitable for use in the invention is given in Table 2 (SEQ ID NO:8).

The terms “antibody” and its plural form “antibodies” refer to whole immunoglobulins and any antigen-binding fragment (“antigen-binding portion”) or single chains thereof Δn “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (C1q) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In an embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The terms “fusion protein” or “fusion polypeptide” refer to proteins that combine the properties of two or more individual proteins. Such proteins have at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order and may include more than one of either or both of the constituent polypeptides. The term encompasses conservatively modified variants, polymorphic variants, alleles, mutants, subsequences, interspecies homologs, and immunogenic fragments of the antigens that make up the fusion protein. Fusion proteins of the disclosure can also comprise additional copies of a component antigen or immunogenic fragment thereof. The fusion protein may contain one or more binding domains linked together and further linked to an Fc domain, such as an IgG Fc domain. Fusion proteins may be further linked together to mimic a monoclonal antibody and provide six or more binding domains. Fusion proteins may be produced by recombinant methods as is known in the art. Preparation of fusion proteins are known in the art and are described, e.g., in International Patent Application Publication Nos. WO 1995/027735 A1, WO 2005/103077 A1, WO 2008/025516 A1, WO 2009/007120 A1, WO 2010/003766 A1, WO 2010/010051 A1, WO 2010/078966 A1, U.S. Patent Application Publication Nos. US 2015/0125419 A1 and US 2016/0272695 A1, and U.S. Pat. No. 8,921,519, the disclosures of each of which are incorporated by reference herein.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “conservative amino acid substitutions” in means amino acid sequence modifications which do not abrogate the binding of an antibody or fusion protein to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an antibody is preferably replaced with another amino acid residue from the same class. Methods of identifying amino acid conservative substitutions which do not eliminate antigen binding are well-known in the art (see, e.g., Brummell, et al., Biochemistry 1993, 32, 1180-1187; Kobayashi, et al., Protein Eng. 1999, 12, 879-884 (1999); and Burks, et al., Proc. Natl. Acad. Sci. USA 1997, 94, 412-417.

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

Nucleic acid sequences implicitly encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. Batzer, et al., Nucleic Acid Res. 1991, 19, 5081; Ohtsuka, et al., J. Biol. Chem. 1985, 260, 2605-2608; Rossolini, et al., Mol. Cell. Probes 1994, 8, 91-98. The term nucleic acid is used interchangeably with cDNA, mRNA, oligonucleotide, and polynucleotide.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99%, or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

The term “hematological malignancy” refers to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, and non-Hodgkin's lymphomas. The term “B cell hematological malignancy” refers to hematological malignancies that affect B cells.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the lung, breast, prostate, colon, rectum, and bladder. The tissue structure of solid tumors includes interdependent tissue compartments including the parenchyma (cancer cells) and the supporting stromal cells in which the cancer cells are dispersed and which may provide a supporting microenvironment.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### Systems and Methods for Determining the Beneficial Administration of TILs

As described herein, providing a population of TILs to a target entity having a condition, can lead to a discernable effect on the condition, provided that the target entity has a first property. Determining whether such target entity does in fact possess such property can be of interest for determining whether providing the population of TILs to the target entity is warranted or not, because the lack of the first property would indicate that it is not. In order to determine whether such first property is present, the target entity can be classified into a time-to-event class. In some embodiments, a time-to-event class is associated with a certain likelihood that the target entity has the first property.

In some embodiments, the target entity can be a patient having cancer, for example a mammal, or more specifically a human. In some embodiments, the condition associated with the target entity is a disease or disorder, for example cancer. In some embodiments, the first property is the ability of the target entity to respond in a certain way to administration of T cells, for example by exhibiting a discernable effect on its condition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the first property is the ability of the target entity to respond in a certain way to administration of TILs, for example by exhibiting a discernable effect on its condition. In some embodiments, the discernable effect on the condition is remission of the condition, for example remission of cancer, such as complete remission or partial remission, or lack of progression of the condition for a period of time, for example lack of cancer progression. In some embodiments, the event is a change in the status of the target entity, for example renewed progression of the condition. In some embodiments, the discernable effect is a complete response, a partial response, no response, stable disease, or progressive disease.

The first property of the target entity can be determined from samples of the target entity, for example biological samples from a human. In some embodiments, the first property of the target can be determined by comparing a sample of the target entity with samples of other entities which have been provided T cells in the past, and on which entities a discernable effect of providing T cells, or lack thereof, is known. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells. In some embodiments, the first property of the target can be determined by comparing a sample of the target entity with samples of other entities which have been provided TILs in the past, and on which entities a discernable effect of providing TILs, or lack thereof, is known. The samples of these other entities can be used to build time-to-event classes. In some embodiments, the samples of these other entities were collected prior to the providing of TILs. In some embodiments, the other entities had conditions such as metastatic melanoma.

In some embodiments, the samples of both the target entity, and the samples of the other entities, are used to generate an analytical signature prior to comparison. In some embodiments, the analytical signature comprises one or more features. In some embodiments, the analytical signature is derived from electrophoresis or chromatography data. As described herein, in some embodiments, the analytical signature is derived from mass spectra data. In some embodiments, the mass spectra data is derived from MALDI mass spectra, for example MALDI-TOF data. In some embodiments, the analytical signature includes selected m/z values from the mass spectra data. Through various mass spectra processing techniques described herein, the one or more features of the analytical signature are derived from the mass spectra data. In some embodiments, the features manifest themselves in specific m/z regions of the spectra where spectral peaks change in intensity and shape. In some embodiments, such features are defined by certain m/z ranges. In some embodiments, the m/z ranges comprise an m/z range left limit. In some embodiments, the m/z ranges comprise an m/z range center. In some embodiments, the m/z ranges comprise an m/z range right limit. In some embodiments, the feature is assigned a value. In some embodiments, the feature value for a specific spectrum is the area under the spectrum within the m/z span of the feature definition. In some embodiments, the feature definition is according to the ranges described in Table 16.

In one embodiment, the invention provides a system for screening a target entity to determine whether it has a first property, the system comprising: at least one processor and memory addressable by the at least one processor, the memory storing at least one program for execution by the at least one processor, the at least one program comprising instructions for: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property comprises a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method for screening a target entity to determine whether it has a first property, the method comprising: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property comprises a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a system for screening a target entity to determine whether it has a first property, the system comprising: at least one processor and memory addressable by the at least one processor, the memory storing at least one program for execution by the at least one processor, the at least one program comprising instructions for: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property comprises a discernable effect of providing a population of tumor infiltrating lymphocytes (TILs) on a condition associated with the first entity.

In one embodiment, the invention provides a method for screening a target entity to determine whether it has a first property, the method comprising: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property comprises a discernable effect of providing a population of tumor infiltrating lymphocytes (TILs) on a condition associated with the first entity.

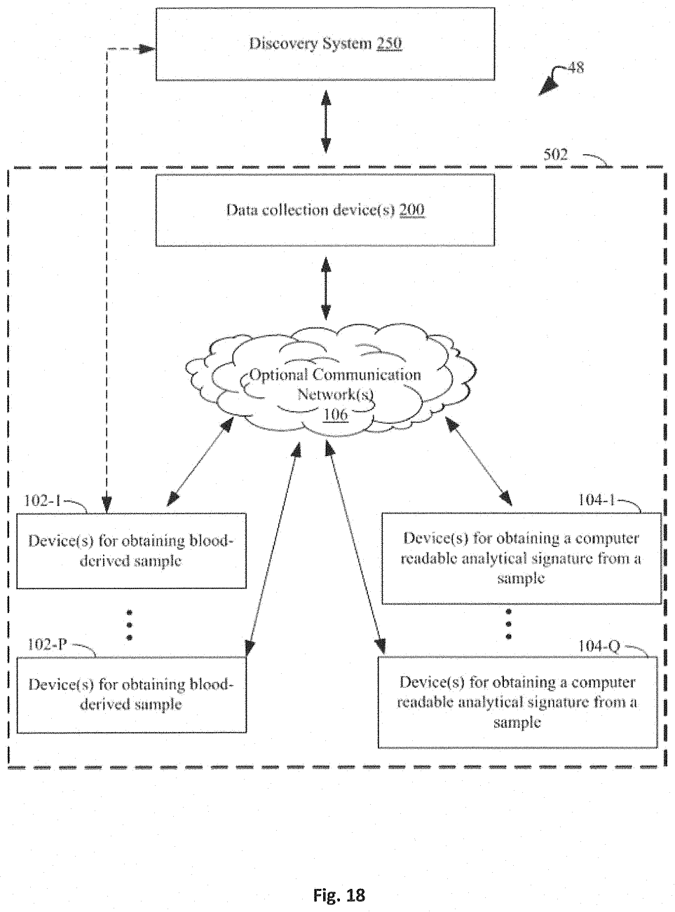

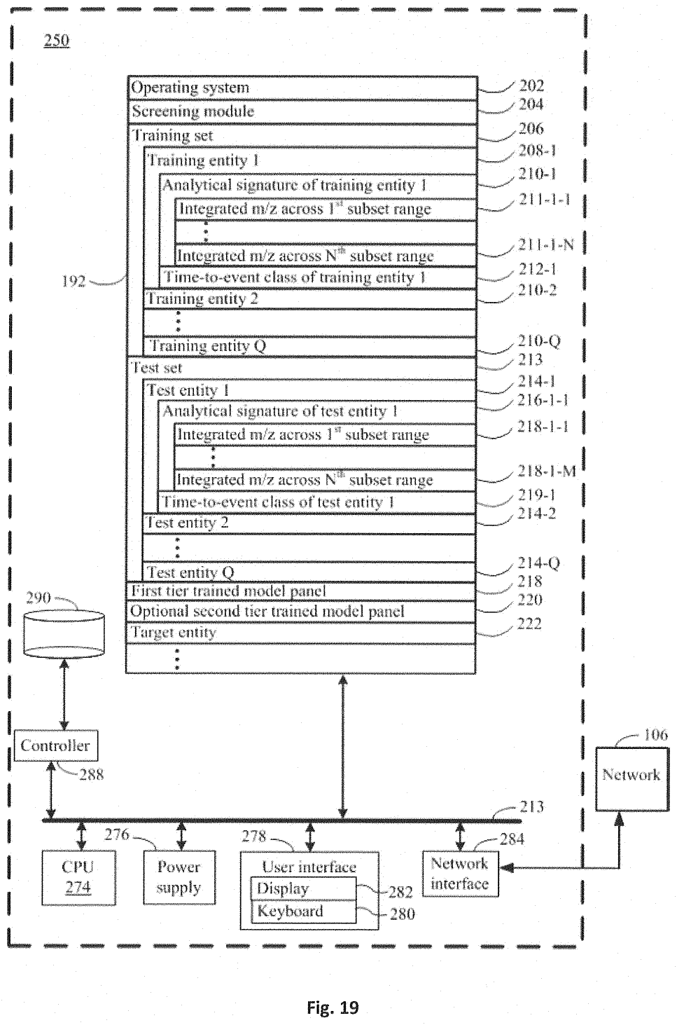

A detailed description of a system 48 for screening a target entity to determine whether it has a first property in accordance with the present disclosure is described in conjunction with FIGS. 18 through 20. As such, FIGS. 18 through 20 collectively illustrate the topology of the system in accordance with the present disclosure. In the topology, there is a discovery system for screening a target entity to determine whether it has a first property (“discovery system 250”) (FIGS. 18, and 19), one or more data collection devices 200, devices for obtaining blood-derived samples 102, and devices for obtaining computer readable analytical signatures from such samples 104 (FIG. 18). Throughout the present disclosure, the data collection devices 200 and the discovery system 250 will be referenced as separate devices solely for purposes of clarity. That is, the disclosed functionality of the data collection device 200 and the disclosed functionality of the discovery system 250 are contained in separate devices as illustrated in FIG. 18. However, it will be appreciated that, in fact, in some embodiments, the disclosed functionality of the one or more data collection devices 200 and the disclosed functionality of the discovery system 250 are contained in a single device. Likewise, in some embodiments, the data collection device 200 and the devices for obtaining blood-derived samples 102 and/or the devices for obtaining computer readable analytical signatures from such samples 104 are the same devices.

Referring to FIG. 18, the discovery system 250 screens a target entity to determine whether it has a first property. To do this, the data collection device 200, which is in electrical communication with the discovery system 250, A) acquires a first computer readable analytical signature from a sample of the target entity at a first time point, inputs the first computer readable analytical signature of the target entity into a first tier trained model panel thereby obtaining a first trained model output value for the entity, and C) classifies the target entity based upon the first trained model output value with a time-to-event class in an enumerated set of time-to-event classes. Each respective time-to-event class in the enumerated set of time-to-event classes is associated with a different likelihood that the target entity has the first property. Moreover, the first property includes a discernable effect of providing a population of tumor infiltrating lymphocytes (TILs) on a condition associated with the first entity. In some embodiments, the first property includes a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the data collection device 200 receives such data directly from the device(s) 102 and the device(s) 104. For instance, in some embodiments the data collection device 200 receives this data wirelessly through radio-frequency signals. In some embodiments such signals are in accordance with an 802.11 (WiFi), Bluetooth, ZigBee, or by RFID communication. In some embodiments, the data collection device 200 receives such data directly, analyzes the data, and passes the analyzed data to the discover system 250.

In some embodiments, the data collection device 200 and/or the discovery system 250 is not proximate to the devices 102 and/or devices 104 and/or does not have direct wireless capabilities or such wireless capabilities are not used for the purpose of acquiring data. In such embodiments, a communication network 106 may be used to communicate measurements of the first computer readable analytical signature (and/or second computer readable analytical signatures) from the devices 102 and the devices 104 to the data collection device 200 and/or the discovery system 250.

Examples of networks 106 include, but are not limited to, the World Wide Web (WWW), an intranet and/or a wireless network, such as a cellular telephone network, a local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 48 are possible. For instance, rather than relying on a communications network 106, the one or more devices 102 and the one or more devices 104 may wirelessly transmit information directly to the data collection device 200 and/or discovery system 250. Further, the data collection device 200 and/or the discovery system 250 may constitute a portable electronic device, a server computer, or in fact constitute several computers that are linked together in a network or be a virtual machine in a cloud computing context. As such, the exemplary topology shown in FIG. 18 merely serves to describe the features of an embodiment of the present disclosure in a manner that will be readily understood to one of skill in the art.

Referring to FIG. 19, in typical embodiments, the discovery system 250 comprises one or more computers. For purposes of illustration in FIG. 19, the discovery system 250 is represented as a single computer that includes all of the functionality for screening a target entity to determine whether it has a first property. However, the disclosure is not so limited. In some embodiments, the functionality for screening a target entity to determine whether it has a first property is spread across any number of networked computers and/or resides on each of several networked computers and/or is hosted on one or more virtual machines at a remote location accessible across the communications network 106. One of skill in the art will appreciate that any of a wide array of different computer topologies are used for the application and all such topologies are within the scope of the present disclosure.

Turning to FIG. 19 with the foregoing in mind, an exemplary discovery system 250 for screening a target entity to determine whether it has a first property comprises one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the discovery system 250 but that can be electronically accessed by the discovery system 250 over an Internet, intranet, or other form of network or electronic cable (illustrated as element 106 in FIG. 19) using network interface 284.

In some embodiments, the memory 192 of the discovery system 250 for screening a target entity to determine whether it has a first property stores:
an operating system 202 that includes procedures for handling various basic system services;
a screening module 204 for screening a target entity to determine whether it has a first property;
a training set 206 that comprises an analytical signature 210 for each training entity 208 in a plurality of training entities and, for each respective analytical signature, (i) one or more integrated m/z 211 across a different independent subset range of an m/z spectra obtained by mass spectrometry from a sample from the corresponding training entity and (ii) a time-to-event class 212 of the training entity 208;
a test set 213 that comprises an analytical signature 216 for each test entity 214 in a plurality of test entities and, for each respective analytical signature 216, (i) one or more integrated m/z 218 across a different independent subset range of an m/z spectra obtained by mass spectrometry from a sample from the corresponding test entity and (ii) a time-to-event class 219 of the test entity 214;
a first tier trained model panel 218 for screening a target entity to determine whether it has a first property;
an optional second tier trained model panel 220 for screening a target entity to determine whether it has a first property; and
data for a target entity 222 including an analytical signature for the target entity.

In some embodiments, the screening module 204 is accessible within any browser (phone, tablet, laptop/desktop). In some embodiments, the screening module 204 runs on native device frameworks, and is available for download onto the discovery system 250 running an operating system 202 such as Android or iOS.

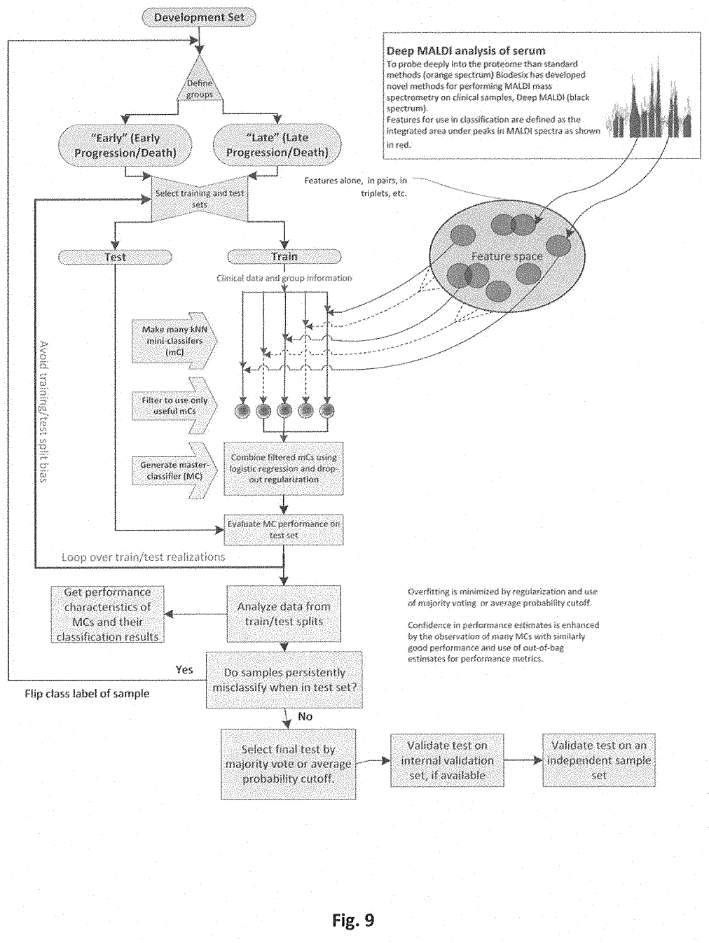

In some embodiments, the training set 206 is the training set referenced in FIG. 9. In some embodiments, the test set 213 is the test set referenced in FIG. 9.

In some embodiments, the first tier trained model panel consists of a single support vector machine. In some embodiments, the first tier trained model panel consists of a plurality of support vector machines.

In some embodiments, the target entity is a live entity, such as a mammal. In some embodiments, the target entity is an animal, for example a farm animal or a companion animal such as a pet. In some embodiments, the target entity is a human. In some embodiments, the target entity is a patient having a diseases or disorder. In some embodiments, the target entity is a female. In some embodiments, the target entity is a male. In some embodiments, the target entity is white or Caucasian. In some embodiments, the target entity is Black or African-american. In some embodiments, the target entity is Asian. In some embodiments, the target entity is multiracial. In some embodiments, the diseases or disorder is a cancer described herein.

In some embodiments, the target entity can have any age. In some embodiments, the target entity is between about 1 year old, and about 5 years old. In some embodiments, the target entity is between about 3 years old, and about 10 years old. In some embodiments, the target entity is between about 5 years old, and about 15 years old. In some embodiments, the target entity is between about 7 years old, and about 18 years old. In some embodiments, the target entity is between about 12 years old, and about 20 years old. In some embodiments, the target entity is between about 16 years old, and about 25 years old. In some embodiments, the target entity is between about 20 years old, and about 35 years old. In some embodiments, the target entity is between about 33 years old, and about 45 years old. In some embodiments, the target entity is between about 40 years old, and about 55 years old. In some embodiments, the target entity is between about 48 years old, and about 65 years old. In some embodiments, the target entity is between about 50 years old, and about 70 years old. In some embodiments, the target entity is between about 60 years old, and about 80 years old. In some embodiments, the target entity is between about 70 years old, and about 90 years old. In some embodiments, the target entity is more than 85 years old.

In some embodiments, the target entity is about 1 year old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, about 17 years old, about 18 years old, about 19 years old, about 20 years old, about 21 years old, about 22 years old, about 23 years old, about 24 years old, about 25 years old, about 26 years old, about 27 years old, about 28 years old, about 29 years old, about 30 years old, about 31 years old, about 32 years old, about 33 years old, about 34 years old, about 35 years old, about 36 years old, about 37 years old, about 38 years old, about 39 years old, about 40 years old, about 41 years old, about 42 years old, about 43 years old, about 44 years old, about 45 years old, about 46 years old, about 47 years old, about 48 years old, about 49 years old, about 50 years old, about 51 years old, about 52 years old, about 53 years old, about 54 years old, about 55 years old, about 56 years old, about 57 years old, about 58 years old, about 59 years old, about 60 years old, about 61 years old, about 62 years old, about 63 years old, about 64 years old, about 65 years old, about 66 years old, about 67 years old, about 68 years old, about 69 years old, about 70 years old, about 71 years old, about 72 years old, about 73 years old, about 74 years old, about 75 years old, about 76 years old, about 77 years old, about 78 years old, about 79 years old, about 80 years old, about 81 years old, about 82 years old, about 83 years old, about 84 years old, about 85 years old, about 86 years old, about 87 years old, about 88 years old, about 89 years old, about 90 years old, about 91 years old, about 92 years old, about 93 years old, about 94 years old, about 95 years old, about 96 years old, about 97 years old, about 98 years old, about 99 years old, or about 100 years old.

In some embodiments, the sample of the entity is any sample of a tissue or bodily fluid of the entity. In some embodiments, the sample of the entity is a blood sample or a lymph sample from the entity. In some embodiments, the sample of the entity is a serum sample or a plasma sample from the entity. In some embodiments, the sample of the entity is a tumor sample, for example a cancer tumor sample. In some embodiments, the sample is a pre-treatment sample, a post-treatment sample, or a sample obtained during treatment.

In some embodiments, the condition is a disease or disorder. In some embodiments, the condition is cancer. In some embodiments, the condition is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the condition is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁻/HER2⁺), triple negative breast cancer (ER⁻/PR⁻/HER2⁻), double-refractory melanoma, and uveal (ocular) melanoma.

In some embodiments, the acquiring comprises acquiring values of selected m/z of the sample using a spectrometer. In some embodiments, the acquiring comprises acquiring integrated values of selected m/z of the sample across each subset in a plurality of predetermined subsets of m/z ranges using a spectrometer thereby forming the first computer readable analytical signature. In some embodiments, each subset in the plurality of predetermined subsets of m/z ranges is selected from Table 16. In some embodiments, the acquiring comprises acquiring values of selected m/z of the sample using a mass-spectrometer conducted in positive ion mode.

In some embodiments, each subset in the first plurality of predetermined subsets of m/z ranges is correlated or anti-correlated with the complement system protein functional group, the acute inflammation protein functional group, the acute response protein functional group, or the acute phase protein functional group. In some embodiments, each subset in the first plurality of predetermined subsets of m/z ranges is correlated or anti-correlated with a level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In some embodiments, the acquiring A) comprises acquiring integrated m/z values of the sample across each respective subset in a plurality of predetermined subsets of m/z ranges using a spectrometer thereby forming the first computer readable analytical signature, the first tier trained model panel comprises a plurality of first master-classifiers; and the inputting the first computer readable analytical signature of the entity into the first tier trained model panel comprises: (i) providing each respective first master-classifier in the plurality of first master-classifiers with the first computer readable analytical signature thereby obtaining a corresponding first component output value of the respective first master-classifier in a plurality of first component output values, and (ii) combining the plurality of first component output values to form the first trained model output value for the entity. In some embodiments, the at least one program further includes instructions for: applying a cutoff threshold to each first component output value in the plurality of first component output values prior to the combining (ii), and the combining the plurality of first component output values to form the first trained model output value for the target entity (ii) comprises an unweighted voting across the plurality of first component output values to form the first trained model output value for the target entity. In some embodiments, a respective first master-classifier in the plurality of first master-classifiers comprises a logistic expression of a plurality of mini-classifiers, and each respective mini-classifier in the plurality of mini-classifiers contributes to the logistic expression using a unique subset of the plurality of predetermined subsets of m/z ranges that corresponds to the respective mini-classifier. In some embodiments, each respective mini-classifier in the plurality of mini-classifiers contributes to the logistic expression by applying the unique subset of the plurality of predetermined subsets of m/z ranges that corresponds to the respective mini-classifier against a different test set associated with the first master-classifier using nearest neighbor analysis, and the different test set comprises a first plurality of test entities, and for each respective test entity in the first plurality of test entities, (i) measured values across each m/z subset in the plurality of predetermined subsets of m/z ranges from a test sample from the respective test entity and (ii) a specified time-to-event class in the enumerated set of time-to-event classes for the respective test entity. In some embodiments, the nearest neighbor analysis is k-nearest neighbor analysis, wherein k is a positive integer.

In some embodiments, each respective first master-classifier in the plurality of first master-classifiers comprises a different logistic expression of a different plurality of mini-classifiers, and each respective mini-classifier in the different plurality of mini-classifiers for a respective first master-classifier in the plurality of first master-classifiers contributes to the corresponding logistic expression by applying a unique subset of the plurality of predetermined subsets of m/z ranges that corresponds to the respective mini-classifier against a different test set, in a plurality of test sets, wherein the different test set is associated with the respective first master-classifier, using nearest neighbor analysis, and the different test set associated with the respective first master-classifier comprises a respective plurality of test entities, and for each respective test entity in the respective plurality of test entities, (i) measured integrated m/z values of a test sample from a respective test entity in the respectively plurality of test entities across each respective subset in the plurality of predetermined subsets of m/z ranges and (ii) a specified time-to-event class in the enumerated set of time-to-event classes. In some embodiments, there is partial overlap between each respective test set in the plurality of test sets.

In some embodiments, each predetermined subset of m/z ranges in the plurality of predetermined subsets of m/z ranges is centered on an m/z value provided in column one of Table 21. In some embodiments, at least 10 predetermined subsets of m/z ranges in the plurality of predetermined subsets of m/z ranges is centered on a different m/z value provided in column one of Table 21. In some embodiments, at least 15 predetermined subsets of m/z ranges in the plurality of predetermined subsets of m/z ranges is centered on a different m/z value provided in column one of Table 21. In some embodiments, at least 20 predetermined subsets of m/z ranges in the plurality of predetermined subsets of m/z ranges is centered on a different m/z value provided in column one of Table 21. In some embodiments, at least 25 predetermined subsets of m/z ranges in the plurality of predetermined subsets of m/z ranges is centered on a different m/z value provided in column one of Table 21. In some embodiments, at least 30 predetermined subsets of m/z ranges in the plurality of predetermined subsets of m/z ranges is centered on a different m/z value provided in column one of Table 21. In some embodiments, at least 35 predetermined subsets of m/z ranges in the plurality of predetermined subsets of m/z ranges is centered on a different m/z value provided in column one of Table 21. In some embodiments, at least 40 predetermined subsets of m/z ranges in the plurality of predetermined subsets of m/z ranges is centered on a different m/z value provided in column one of Table 21. In some embodiments, at least 45 predetermined subsets of m/z ranges in the plurality of predetermined subsets of m/z ranges is centered on a different m/z value provided in column one of Table 21. In some embodiments, at least 50 predetermined subsets of m/z ranges in the plurality of predetermined subsets of m/z ranges is centered on a different m/z value provided in column one of Table 21. In some embodiments, at least 55 predetermined subsets of m/z ranges in the plurality of predetermined subsets of m/z ranges is centered on a different m/z value provided in column one of Table 21. In some embodiments, at least 60 predetermined subsets of m/z ranges in the plurality of predetermined subsets of m/z ranges is centered on a different m/z value provided in column one of Table 21. In some embodiments, at least 65 predetermined subsets of m/z ranges in the plurality of predetermined subsets of m/z ranges is centered on a different m/z value provided in column one of Table 21. In some embodiments, at least 70 predetermined subsets of m/z ranges in the plurality of predetermined subsets of m/z ranges is centered on a different m/z value provided in column one of Table 21. In some embodiments, at least 75 predetermined subsets of m/z ranges in the plurality of predetermined subsets of m/z ranges is centered on a different m/z value provided in column one of Table 21. In some embodiments, at least 80 predetermined subsets of m/z ranges in the plurality of predetermined subsets of m/z ranges is centered on a different m/z value provided in column one of Table 21. In some embodiments, at least 85 predetermined subsets of m/z ranges in the plurality of predetermined subsets of m/z ranges is centered on a different m/z value provided in column one of Table 21. In some embodiments, at least 90 predetermined subsets of m/z ranges in the plurality of predetermined subsets of m/z ranges is centered on a different m/z value provided in column one of Table 21. In some embodiments, at least 95 predetermined subsets of m/z ranges in the plurality of predetermined subsets of m/z ranges is centered on a different m/z value provided in column one of Table 21. In some embodiments, at least 100 predetermined subsets of m/z ranges in the plurality of predetermined subsets of m/z ranges is centered on a different m/z value provided in column one of Table 21. In some embodiments, at least 105 predetermined subsets of m/z ranges in the plurality of predetermined subsets of m/z ranges is centered on a different m/z value provided in column one of Table 21. In some embodiments, at least 110 predetermined subsets of m/z ranges in the plurality of predetermined subsets of m/z ranges is centered on a different m/z value provided in column one of Table 21. In some embodiments, at least 115 predetermined subsets of m/z ranges in the plurality of predetermined subsets of m/z ranges is centered on a different m/z value provided in column one of Table 21. In some embodiments, at least 120 predetermined subsets of m/z ranges in the plurality of predetermined subsets of m/z ranges is centered on a different m/z value provided in column one of Table 21. In some embodiments, at least 125 predetermined subsets of m/z ranges in the plurality of predetermined subsets of m/z ranges is centered on a different m/z value provided in column one of Table 21. In some embodiments, at least 130 predetermined subsets of m/z ranges in the plurality of predetermined subsets of m/z ranges is centered on a different m/z value provided in column one of Table 21. In some embodiments, at least 135 predetermined subsets of m/z ranges in the plurality of predetermined subsets of m/z ranges is centered on a different m/z value provided in column one of Table 21. In some embodiments, at least 140 predetermined subsets of m/z ranges in the plurality of predetermined subsets of m/z ranges is centered on a different m/z value provided in column one of Table 21. In some embodiments, at least 145 predetermined subsets of m/z ranges in the plurality of predetermined subsets of m/z ranges is centered on a different m/z value provided in column one of Table 21. In some embodiments, at least 150 predetermined subsets of m/z ranges in the plurality of predetermined subsets of m/z ranges is centered on a different m/z value provided in column one of Table 21.

In one embodiment, the invention provides a system for screening a target entity to determine whether it has a first property, the system comprising: at least one processor and memory addressable by the at least one processor, the memory storing at least one program for execution by the at least one processor, the at least one program comprising instructions for: A) acquiring a first computer readable analytical signature from a sample of the target entity at a first time point; B) inputting the first computer readable analytical signature of the target entity into a first tier trained model panel thereby obtaining a first trained model output value for the entity; and C) classifying the target entity based upon the first trained model output value with a time-to-event class in an enumerated set of time-to-event classes, wherein each respective time-to-event class in the enumerated set of time-to-event classes is associated with a different likelihood that the target entity has the first property, wherein the first property comprises a discernable effect of providing a population of tumor infiltrating lymphocytes (TILs) on a condition associated with the first entity; wherein the acquiring A) comprises: acquiring integrated m/z values of the sample across each respective subset in a first plurality of predetermined subsets of m/z ranges thereby forming the first computer readable analytical signature, and acquiring integrated m/z values of the sample across each respective subset in a second plurality of predetermined subsets of m/z ranges thereby forming a second computer readable analytical signature, and the classifying C) comprises: classifying the target entity with a first time-to-event class in the enumerated set of time-to-event classes when the first trained model output value is in a first value range; and performing a follow up procedure when the first trained model output value is in a second value range; wherein the follow up procedure comprises: i) inputting the second computer readable analytical signature of the target entity into a second tier trained model panel thereby obtaining a second trained model output value for the entity; and ii) classifying the target entity based upon the second trained model output value with a time-to-event class in the enumerated set of time-to-event classes. In other embodiments, the first property comprises a discernable effect of providing a population of T cells on a condition associated with the first entity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the first tier trained model panel comprises a plurality of first master-classifiers; and the inputting the first computer readable analytical signature of the target entity into the first tier trained model panel comprises: (i) providing each respective first master-classifier in the plurality of first master-classifiers with the first computer readable analytical signature thereby obtaining a corresponding first component output value of the respective first master-classifier in a plurality of first component output values, and (ii) combining the plurality of first component output values to form the first trained model output value for the entity. In some embodiments, the second tier trained model panel comprises a plurality of second master-classifiers; and the inputting the second computer readable analytical signature of the target entity into the second tier trained model panel comprises: (i) providing each respective second master-classifier in the plurality of second master-classifiers with the second computer readable analytical signature thereby obtaining a corresponding second component output value of the respective second master-classifier in a plurality of second component output values, and (ii) combining the plurality of second component output values to form the second trained model output value for the entity. In some embodiments, the at least one program further comprises instructions for: applying a cutoff threshold to each second component output value in the plurality of second component output values prior to the combining the plurality of second component output values (ii), and the combining the plurality of second component output values to form the second trained model output value for the entity (ii) comprises an unweighted voting across the plurality of second component output values to form the second trained model output value for the entity. In some embodiments, a respective first master-classifier in the plurality of first master-classifiers comprises a first logistic expression of the first plurality of mini-classifiers, each respective mini-classifier in the first plurality of mini-classifiers contributes to the first logistic expression using a unique subset of the plurality of predetermined subsets of m/z ranges that corresponds to the respective mini-classifier, a respective second master-classifier in the plurality of second master-classifiers comprises a second logistic expression of the second plurality of mini-classifiers, and each respective mini-classifier in the second plurality of mini-classifiers contributes to the second logistic expression using a unique subset of the plurality of predetermined subsets of m/z ranges that corresponds to the respective mini-classifier. In some embodiments, each respective mini-classifier in the first plurality of mini-classifiers contributes to the first logistic expression by applying the unique subset of the plurality of predetermined subsets of m/z ranges that corresponds to the respective mini-classifier against a different test set associated with the first master-classifier using nearest neighbor analysis, the different test set comprises a first plurality of test entities, and for each respective test entity in the first plurality of test entities, (i) measured values for the selected m/z of a test sample from the respective test entity at each respective subset in the plurality of predetermined subsets of m/z ranges and (ii) a specified time-to-event class in the enumerated set of time-to-event classes, each respective mini-classifier in the second plurality of mini-classifiers contributes to the second logistic expression by applying the unique subset of the plurality of predetermined subsets of m/z ranges that corresponds to the respective mini-classifier against a different test set associated with the second master-classifier using nearest neighbor analysis, the different test set comprises a second plurality of test entities, and for each respective test entity in the second plurality of test entities, (i) measured values for the selected m/z of a test sample from the respective test entity at each respective subset in the plurality of predetermined subsets of m/z ranges and (ii) a specified time-to-event class in the enumerated set of time-to-event classes. In some embodiments, the nearest neighbor analysis is k-nearest neighbor analysis, wherein k is a positive integer. In some embodiments, each respective first master-classifier in the plurality of first master-classifiers comprises a different logistic expression of a different plurality of mini-classifiers, and each respective mini-classifier in the different plurality of mini-classifiers for a respective first master-classifier in the plurality of first master-classifiers contributes to the first logistic expression by applying a unique subset of the plurality of predetermined subsets of m/z ranges that corresponds to the respective mini-classifier against a different test set, in a first plurality of test sets, wherein the different test set is associated with the respective first master-classifier using nearest neighbor analysis, the different test set associated with the respective first master-classifier comprises a respective plurality of test entities, and for each respective test entity in the plurality of test entities, (i) measured values for the selected m/z of a test sample from a respective test entity in the respectively plurality of test entities at each respective subset in the plurality of predetermined subsets of m/z ranges and (ii) a specified time-to-event class in the enumerated set of time-to-event classes, each respective second master-classifier in the plurality of second master-classifiers comprises a different logistic expression of a different plurality of mini-classifiers, and each respective mini-classifier in the different plurality of mini-classifiers for a respective second master-classifier in the plurality of second master-classifiers contributes to the second logistic expression by applying a unique subset of the plurality of predetermined subsets of m/z ranges that corresponds to the respective mini-classifier against a different test set, in a second plurality of test sets, wherein the different test set is associated with the respective second master-classifier, using nearest neighbor analysis, the different test set associated with the respective second master-classifier comprises a respective plurality of test entities, and for each respective test entity in the respective plurality of test entities, (i) measured values for the selected m/z of a test sample from a respective test entity in the respectively plurality of test entities at each respective subset in the plurality of predetermined subsets of m/z ranges and (ii) a specified time-to-event class in the enumerated set of time-to-event classes.

In some embodiments, each predetermined subset of m/z ranges in the first plurality of predetermined subsets of m/z ranges is centered on an m/z value provided in column one of Table 21, and each predetermined subset of m/z ranges in the second plurality of predetermined subsets of m/z ranges is centered on an m/z value provided in column two of Table 21. In some embodiments, at least 10 predetermined subsets of m/z ranges in the first plurality of predetermined subsets of m/z ranges is centered on a different m/z value provided in column one of Table 21, and at least 4 predetermined subsets of m/z ranges in the second plurality of predetermined subsets of m/z ranges is centered on a different m/z value provided in column two of Table 21. In some embodiments, at least 40 predetermined subsets of m/z ranges in the first plurality of predetermined subsets of m/z ranges is centered on a different m/z value provided in column one of Table 21, and at least 8 predetermined subsets of m/z ranges in the second plurality of predetermined subsets of m/z ranges is centered on a different m/z value provided in column two of Table 21. In some embodiments, at least 80 predetermined subsets of m/z ranges in the first plurality of predetermined subsets of m/z ranges is centered on a different m/z value provided in column one of Table 21, and at least 12 predetermined subsets of m/z ranges in the second plurality of predetermined subsets of m/z ranges is centered on a different m/z value provided in column two of Table 21. In some embodiments, at least 120 predetermined subsets of m/z ranges in the plurality of predetermined subsets of m/z ranges is centered on a different m/z value provided in column one of Table 21, and at least 16 predetermined subsets of m/z ranges in the second plurality of predetermined subsets of m/z ranges is centered on a different m/z value provided in column two of Table 21.

In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 147, 148, 149, or 150 predetermined subsets of m/z ranges in the plurality of predetermined subsets of m/z ranges is centered on a different m/z value provided in column one of Table 21, and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 predetermined subsets of m/z ranges in the second plurality of predetermined subsets of m/z ranges is centered on a different m/z value provided in column two of Table 21.

In some embodiments, the acquiring A) comprises deriving characteristic values of the sample by electrophoresis or chromatography. In some embodiments, the enumerated set of classes consists of good, intermediate, bad, late, early, plus (+), and minus (−). In some embodiments, the enumerated set of classes comprises good, intermediate, bad, late, early, plus (+), and minus (−). In some embodiments, the discernable effect for the good, late, or plus (+) class is progression free existence of the entity for a first epic commencing at the first time point, and the first epic is selected from the group consisting of about 24 months, about 30 months, about 36 months, about 42 months, about 48 months, about 54 months, about 60 months, up to 60 months, and more than 60 months. In some embodiments, the first epic is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, about 30 months, about 31 months, about 32 months, about 33 months, about 34 months, about 35 months, about 36 months, about 37 months, about 38 months, about 39 months, about 40 months, about 41 months, about 42 months, about 43 months, about 44 months, about 45 months, about 46 months, about 47 months, about 48 months, about 49 months, about 50 months, about 51 months, about 52 months, about 53 months, about 54 months, about 55 months, about 56 months, about 57 months, about 58 months, about 59 months, about 60 months, about 61 months, about 62 months, about 63 months, about 64 months, about 65 months, about 66 months, about 67 months, about 68 months, about 69 months, about 70 months, about 71 months, about 72 months, about 73 months, about 74 months, about 75 months, about 76 months, about 77 months, about 78 months, about 79 months, about 80 months, about 81 months, about 82 months, about 83 months, about 84 months, about 85 months, about 86 months, about 87 months, about 88 months, about 89 months, about 90 months, about 91 months, about 92 months, about 93 months, about 94 months, about 95 months, about 96 months, about 97 months, about 98 months, about 99 months, about 100 months, about 101 month, about 102 months, about 103 months, about 104 months, about 105 months, about 106 months, about 107 months, about 108 months, about 109 months, about 110 months, about 111 months, about 112 months, about 113 months, about 114 months, about 115 months, about 116 months, about 117 months, about 118 months, about 119 months, or about 120 months. In some embodiments, the discernable effect for the good, late or plus (+) class occurs with a likelihood that is greater than a predetermined threshold level. In some embodiments, the predetermined threshold level is fifty percent, sixty percent, seventy percent, eighty percent, or ninety percent. In some embodiments, the providing the population of TILs further comprises co-providing another therapy with the population of TILs for the condition. In some embodiments, the providing the population of T cells further comprises co-providing another therapy with the population of T cells for the condition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the at least one program further comprises instructions for: training, prior to the inputting B), one or more models to thereby form the first tier trained model. In some embodiments, the training comprises: obtaining a training set that represents a plurality of training entities, wherein each training entity in the plurality of training entities has the condition and, for each respective training entity, the training set comprises (i) a computer readable analytical signature from a sample of the respective training entity and (ii) an effect that providing the population of TILs had on the condition, and using the training set to train the one or more models thereby forming the first tier trained model panel. In some embodiments, the enumerated set of classes consists of good, intermediate, bad, late, early, plus (+), and minus (−), and the training set comprises a different plurality of training entities for each class in the enumerated set of classes. In some embodiments, the enumerated set of classes comprises good, intermediate, bad, late, early, plus (+), and minus (−), and the training set comprises a different plurality of training entities for each class in the enumerated set of classes. In some embodiments, the training set comprises: a first subset of entities that have been provided TILs and had no condition progression for a first period of time, a second subset of entities that have been provided TILs and had no condition progression for a second period of time, and a third subset of entities that have been provided TILs and had no condition progression for a third period of time. In some embodiments, the first period of time, the second period time and third period of time are each independently selected from the group consisting of about one year, about two years, about three years, about four years, about five years, and more than five years. In some embodiments, the first period of time, the second period time and third period of time are each independently selected from the group consisting of less than 6 months, about 6 months, about 12 months, about 18 months, about 24 months, about 30 months, about 36 months, about 42 months, about 48 months, about 54 months, about 60 months, up to 60 months, and more than 60 months.

In some embodiments, the at least one program further comprises instructions for: training, prior to the inputting B), one or more models to thereby form the first tier trained model. In some embodiments, the training comprises: obtaining a training set that represents a plurality of training entities, wherein each training entity in the plurality of training entities has the condition and, for each respective training entity, the training set comprises (i) a computer readable analytical signature from a sample of the respective training entity and (ii) an effect that providing the population of T cells had on the condition, and using the training set to train the one or more models thereby forming the first tier trained model panel. In some embodiments, the enumerated set of classes consists of good, intermediate, bad, late, early, plus (+), and minus (−), and the training set comprises a different plurality of training entities for each class in the enumerated set of classes. In some embodiments, the enumerated set of classes comprises good, intermediate, bad, late, early, plus (+), and minus (−), and the training set comprises a different plurality of training entities for each class in the enumerated set of classes. In some embodiments, the training set comprises: a first subset of entities that have been provided T cells and had no condition progression for a first period of time, a second subset of entities that have been provided T cells and had no condition progression for a second period of time, and a third subset of entities that have been provided T cells and had no condition progression for a third period of time. In some embodiments, the first period of time, the second period time and third period of time are each independently selected from the group consisting of about one year, about two years, about three years, about four years, about five years, and more than five years. In some embodiments, the first period of time, the second period time and third period of time are each independently selected from the group consisting of less than 6 months, about 6 months, about 12 months, about 18 months, about 24 months, about 30 months, about 36 months, about 42 months, about 48 months, about 54 months, about 60 months, up to 60 months, and more than 60 months. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

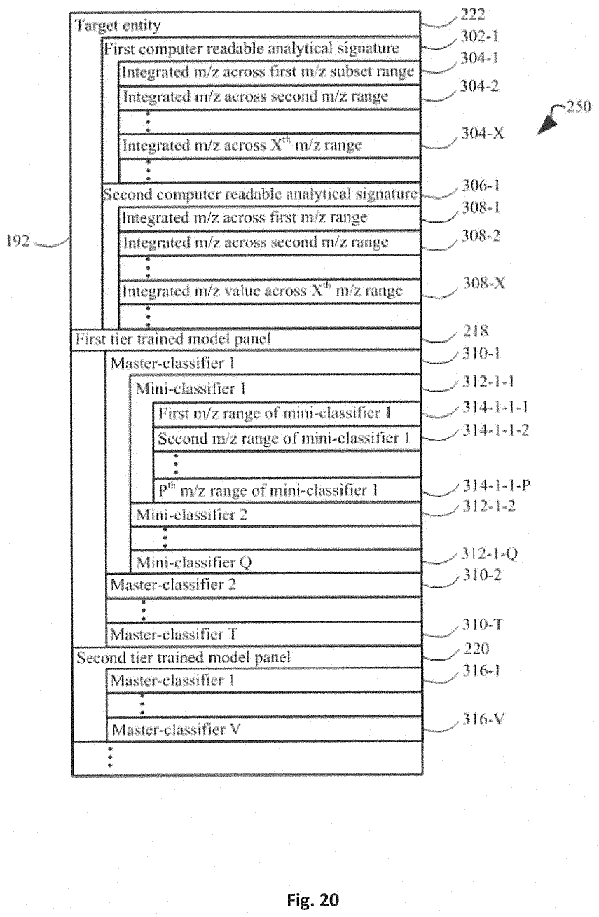

Referring to FIG. 20, in some embodiments, the target entity 222 has a first computer readable analytical signature 302 that comprises a separate integrated m/z value 304 across each respective m/z subset range in a first plurality of m/z subset ranges. For instance, in some embodiments, the first computer readable analytical signature 302 comprises a different subset of m/z ranges for each m/z value provided in column one of Table 21. In such embodiments, the respective m/z value provided in column one of Table 21 is the center value for the subset of m/z ranges and the extent of the range is provided in Table 16. For example, for the feature “3125” listed in the first column of Table 21, a mass spectrograph of a sample from the target entity is integrated between 3118.81 (m/z) and 3130.38 (m/z) as specified in Table 16 (entry number 3: 3118.81, 3124.60, 3130.38) in order to arrive at the integrated m/z value 304 of the target sample from the target entity across the corresponding subset m/z range. Here, the corresponding subset m/z range represents the “feature” and the integrated m/z value of the target sample from the target entity across the corresponding subset m/z range represents the “feature value” for this “feature.”

Referring to FIG. 20, in some embodiments, the target entity 222 has a second computer readable analytical signature 306 that comprises a separate integrated m/z value 308 across each respective m/z subset range in a second plurality of m/z subset ranges. For instance, in some embodiments, the second computer readable analytical signature 302 comprises a different subset of m/z ranges for each m/z value provided in column two of Table 21. In such embodiments, the respective m/z value provided in column two of Table 21 is the center value for the subset of m/z ranges and the extent of the range is provided in Table 16. For example, for the feature “3611” listed in the second column of Table 21, a mass spectrograph of a sample from the target entity is integrated between about 3603.78 m/z and about 3617.35 m/z as specified in Table 16 (entry number 26: 3603.78, 3610.56, 3617.35) in order to arrive at the integrated m/z value 308 of the target sample from the target entity across the corresponding subset m/z range. Here, the corresponding subset m/z range represents the “feature” and the integrated m/z value of the target sample from the target entity across the corresponding subset m/z range represents the “feature value” for this “feature.”

Referring to FIG. 20, in some embodiments, the master-classifier 310 is a single classifier. In some alternative embodiments, the master classifier 310 is a composite of a plurality of mini-classifiers 312. In such embodiments, each mini-classifier 312 comprises, as input, a select number of m/z ranges 314 (subsets). For instance, in some embodiments each m/z range 314 corresponds to one or two of the subset ranges 304 of the first computer readable analytical signature. In some embodiments, each m/z range 314 for a given mini-classifier 314 corresponds to three, four, five, six, seven, eight, nine, or ten of the subset ranges 304 of the first computer readable analytical signature. In some embodiments, each mini-classifier comprises, as input, less than 10 m/z ranges, less than 9 m/z ranges, less than 8 m/z ranges, less than 7 m/z ranges, less than 6 m/z ranges, less than 5 m/z ranges, less than 4 m/z ranges, less than 3 m/z ranges or less than 2 m/z ranges. In some embodiments, each mini-classifier comprises, as input, less than 10 m/z ranges, less than 9 m/z ranges, less than 8 m/z ranges, less than 7 m/z ranges, less than 6 m/z ranges, less than 5 m/z ranges, less than 4 m/z ranges, less than 3 m/z ranges or less than 2 m/z ranges selected from Table 16. In some embodiments each master-classifier is trained using a different subset of the training set 206.

In some embodiments, each master-classifier 310 is a nearest neighbor analysis against the test set 213. That is, select integrated m/z subset ranges in an analytical signature from a target entity 222 serve as input into the first tier trained model panel 218 and/or second tier trained model panel 220 and nearest neighbor analysis is used to determine the most similar entities in the test set 212 to the target entity 222. Then, the time-to-event class of these most similar test entities are polled and combined to form the time-to-event class called by the first tier trained model panel 218 and/or second tier trained model panel 220 for the target entity 222.

In some embodiments, each master-classifier 310 is panel of nearest neighbor analyses against the test set 213. In such embodiments, each nearest neighbor analysis in the panel is a mini-classifier 314. In such embodiments, select integrated m/z subset ranges 314 in an analytical signature 302/306 from the target entity 222 serve as input into each mini-classifier 312 and nearest neighbor analysis is used by each mini-classifier 314 to determine the most similar entities in the test set 213 to the target entity 222. Then, the time-to-event class of these most similar test entities are polled and combined to form the time-to-event class called by each respective master-classifier 310 for the target entity 222.

In some embodiments, the first trained model panel 218 and/or second trained model panel 218 is an artificial neural network. In some embodiments, the first trained model panel 218 and/or second trained model panel 218 is linear regression, non-linear regression, logistic regression, multivariate data analysis, classification using a regression tree, partial least squares projection to latent variables, computation of a neural network, computation of a Bayesian model, computation of a generalized additive model, use of a support vector machine, or modeling comprising boosting or adaptive boosting. See, for example, Duda et al., 2001, Pattern Classification, Second Edition, John Wiley & Sons, Inc., New York; Hastie, 2003, The Elements of Statistical Learning, Springer, New York; and Agresti 1996, An Introduction to Categorical Data Analysis, John Wiley & Sons, New York, each of which is hereby incorporated by reference herein for such purpose.

In some embodiments, the first trained model panel 218 and/or second trained model panel 218 comprises a plurality of mini-classifiers 312 and each respective mini-classifier is an artificial neural network. In some embodiments, the first trained model panel 218 and/or second trained model panel 218 comprises a plurality of mini-classifiers 312 and each respective mini-classifier is a linear regression, non-linear regression, logistic regression, multivariate data analysis, classification using a regression tree, partial least squares projection to latent variables, computation of a neural network, computation of a Bayesian model, computation of a generalized additive model, use of a support vector machine, or modeling comprising boosting or adaptive boosting. See, for example, Duda et al., 2001, Pattern Classification, Second Edition, John Wiley & Sons, Inc., New York; Hastie, 2003, The Elements of Statistical Learning, Springer, New York; and Agresti 1996, An Introduction to Categorical Data Analysis, John Wiley & Sons, New York, each of which is hereby incorporated by reference herein for such purpose. In such embodiments, the mini-classifiers are combined to form a final value for the respective first trained model panel 218 and/or second trained model panel 218

In some implementations, one or more of the above identified data elements or modules of the discovery system 250 for screening a target entity to determine whether it has a first property are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments, the memory 192 and/or 290 stores additional modules and data structures not described above.

In some embodiments, a discovery system 250 for screening a target entity to determine whether it has a first property is a smart phone (e.g., an iPHONE), laptop, tablet computer, desktop computer, or other form of electronic device (e.g., a gaming console). In some embodiments, the discovery system 250 is not mobile. In some embodiments, the discovery system 250 is mobile.

In some embodiments the discovery system 250 is a tablet computer, desktop computer, or other form or wired or wireless networked device. In some embodiments, the discovery system 250 has any or all of the circuitry, hardware components, and software components found in the discovery system 250 depicted in FIG. 18 or 19. In the interest of brevity and clarity, only a few of the possible components of the discovery system 250 are shown in order to better emphasize the additional software modules that are installed on the discovery system 250.

Now that details of a system 48 for screening a target entity to determine whether it has a first property have been disclosed, details regarding aspects of methods for screening a target entity to determine whether it has a first property are disclosed below.

In some embodiments, device 104 is a mass spectrometer. In some embodiments the analytical signature 210 of a reference entity 210, the analytical signature 216 of a test entity 214, and/or the analytical signature 302 or 306 of a target entity is acquired using a mass spectrometer. In some embodiments the analytical signature 210 of a reference entity 210, the analytical signature 216 of a test entity 214, and/or the analytical signature 302 or 306 of a target entity is acquired using a mass spectrometer conducted in positive ion mode. In some embodiments, the analytical signature 210 of a reference entity 210, the analytical signature 216 of a test entity 214, and/or the analytical signature 302 or 306 of a target entity is determined using Deep-MALDI TOF mass spectrometry.

### Deep-MALDI TOF Mass Spectrometry

Deep-MALDI (matrix assisted laser desorption ionization) refers to methods of analyzing biological samples, for example serum or other blood-based samples, using a MALDI-TOF (time of flight) mass spectrometer instrument. The method is described in more detail in U.S. Pat. No. 9,279,798, incorporated herein in its entirety. The method includes the steps of applying the sample to a sample spot on a MALDI-TOF sample plate and directing a large number of laser shots, e.g., more than 20,000, at the sample spot, and collecting mass-spectral data. Any number of laser shots can be used, for example at least 50,000, at least 75,000, at least 100,000, at least 200,000, or at least 500,000 shots are directed onto the sample. Employing a large number of laser shots leads to a reduction in the noise level in the resulting mass spectra, and a significant amount of additional spectral information can be obtained from the sample as compared to traditional MALDI techniques. Furthermore, peaks visible at lower number of shots are better defined and allow for more reliable comparisons between different samples.

In traditional MALDI techniques it is typically difficult to obtain more than 20,000 shots from a single MALDI spot. For example, one issue with using many hundreds of thousands of shots from a MALDI sample spot is that in common spot preparation only some shot locations within a spot yield sufficient ion current to contribute substantially to the signal in a combined spectrum. In deep-MALDI however, specific procedures such as automated raster scanning affords the capability of performing vastly more shots on a single spot than in traditional MALDI techniques. Manual processes to visually select high ion yield locations within a given spot on a MALDI plate for laser shots can be used, but automation of the process to select locations for laser shots is also possible, and preferred for a high throughput implementation. Improving the quality of MALDI spots in such a way that most randomly selected locations yield a high ion current is also an approach that can be used.

Automation of the acquisition may include defining optimal movement patterns of the laser scanning of the spot in a raster fashion, and generation of a specified sequence for multiple raster scans at discrete X/Y coordinate locations within a spot to result in a multitude of shots, e.g., 750,000, 1,000,000, 2,000,000, or 3,000,000 shots from one or more spots. Spectra acquired from 250,000 shots per each of several sample spots can be combined into a 1,000,000 shot spectrum. Hundreds of thousands of shots to millions of shots collected on multiple spots containing the same sample can be averaged together to create one spectrum. Further methods of automation include generation of raster files for non-contiguous X/Y raster scanning of a sample spot, dividing the spot into a grid of sub-spots (e.g., a 3×3 or 5×5 grid), and generating raster files for raster scanning at discrete X/Y coordinate locations of the sub-spots, and using image analysis techniques to identify areas of interest containing relatively high concentrations of sample material for spectral acquisition (multiple shots) and/or those areas where the protein concentration is relatively low, and performing spectral acquisition in the areas with relatively high protein concentration.

Another deep-MALDI technique relates to optimizing the process of sample application to the MALDI plate (“spotting”) to produce uniform, homogeneous crystals of the sample/matrix within a single spot. This process facilitates obtaining hundreds of thousands of shots from a single spot on the MALDI plate using automated methods.

Deep-MALDI has many applications, including biomarker discovery, test development, substance testing, validation of existing tests, and hypothesis generation, e.g., in biomarker discovery efforts. Deep-MALDI also enhances the potential of “dilute and shoot” methods in mass spectrometry research by its ability to reproducibly quantify the amount of many more proteins in a complex sample in a high throughput fashion, as compared to traditional techniques.

### The Diagnostic Cortex

The Diagnostic Cortex refers to methods and systems for classifier generation including obtaining data for classification of a multitude of samples, the data for each of the samples consisting of a multitude of physical measurement feature values and a class label. The methods and their application are described in more detail in U.S. Pat. Nos. 7,736,905, 8,914,238, 8,718,996, 7,858,389, 7,858,390, and 9,477,906, and U.S. Patent Application Publications No. 2011/0208433, and 2013/0344111, incorporated herein in their entireties. Individual mini-classifiers are generated using sets of features from the samples. The performance of the mini-classifiers is tested, and those that meet a performance threshold are retained. A master classifier is then generated by conducting a regularized ensemble training of the retained/filtered set of mini-classifiers to the classification labels for the samples, e.g., by randomly selecting a small fraction of the filtered mini-classifiers (drop out regularization) and conducting logistical training on such selected mini-classifiers. The set of samples are randomly separated into a test set and a training set. The steps of generating the mini-classifiers, filtering and generating a master classifier are repeated for different realizations of the separation of the set of samples into test and training sets, thereby generating a plurality of master classifiers. A final classifier is defined from one or a combination of more than one of the master classifiers.

In contrast to standard applications of machine learning focusing on developing classifiers when large training data sets are available, i.e., the big data challenge, in bio-life-sciences the problem setting is different. Typically, the problem is that the number (n) of available samples, arising typically from clinical studies, is often limited, and the number of attributes (measurements) (p) per sample usually exceeds the number of samples. Rather than obtaining information from many instances, in these deep data problems one attempts to gain information from a deep description of individual instances. The methods involved in the Diagnostic Cortex take advantage of this insight, and are particularly useful in problems where p>>n.

Methods for generating a classifier include a step of obtaining physical measurement data for classification from a plurality of samples (e.g., blood, tissue, or other type of biological sample). The data for classification for each of the samples consists of a multitude of feature values (e.g., integrated intensity values at particular m/Z ranges in mass spectrometry data, fluorescence intensity measurements associated with mRNA transcript, protein, or gene expression levels) and an associated class or group label. The class or group label can take various forms, and it can be iteratively defined in generation of the classifier, and in some embodiments may have some diagnostic or therapeutic meaning or attribute. Further steps include constructing a multitude of individual mini-classifiers using sets of feature values from the samples up to a pre-selected feature set size (s, integer). For example, mini-classifiers are constructed for individual features (s=1) and/or pairs of features (s=2). For example, if the initial feature set contains 100 features, the number of mini-classifiers for s=1 would be 100, and for s=2 would be 4950=100*99/2. The mini-classifiers execute a classification algorithm, such as k-nearest neighbors, in which the values for a feature or pairs of features of a sample instance are compared to the values of the same feature or features in a training set and the nearest neighbors (e.g., k=5) in feature space are identified and by majority vote a class label is assigned to the sample instance by each mini-classifier. Other supervised classification methods could be used as an alternative to k-nearest neighbors, e.g., tree-based classification, linear discriminants, support vector machines, etc. It will be understood that one could use larger values of s, and the number of possible feature combinations would increase resulting in larger computational resource requirements. Further steps include testing the performance of individual mini-classifiers to classify at least some of the multitude of biological samples (e.g., a training set, a subset of an entire development set), and retaining only those mini-classifiers whose classification accuracy or predictive power, or any suitable other performance metric, exceeds a pre-defined threshold, to thereby arrive at a filtered (pruned) set of mini-classifiers. Other steps include generating a master classifier by combining the filtered mini-classifiers using a regularized combination method. This regularized combination method can take, in some embodiments, the form of repeatedly conducting a logistic training of the filtered set of mini-classifiers to the class labels for the samples, which can be done by randomly selecting a small fraction of the filtered mini-classifiers as a result of carrying out an extreme dropout from the filtered set of mini-classifiers (a technique referred to as drop-out regularization), and conducting logistical training on such selected mini-classifiers. Further steps include randomly separating the samples into a test set and a training set, and repeating the previous steps in a programmed computer for different realizations of the separation of the set of samples into test and training sets, thereby generating a plurality of master classifiers, one for each realization of the separation of the set of samples into training and test sets. The methods also include defining a final classifier from one or a combination of more than one of the plurality of master classifiers, final classifier which can be defined in a variety of ways, including by selection of a single master classifier from the plurality of master classifiers having typical or representative performance, by majority vote of all the master classifiers, by modified majority vote, by weighted majority vote, or otherwise.

### T Cells and TILs in Personalized Cancer Treatments

In one embodiment, the invention provides a method of predicting whether a cancer patient is likely to benefit from administration of a population of T cells, either alone or in addition to another anti-cancer therapy, comprising the steps of: obtaining an analytical signature of a blood-derived sample from the patient; and determining that the analytical signature is correlated or anti-correlated with a biological marker that correlates or anti-correlates with the likelihood of the patent to benefit from such administration. In some embodiments, such likelihood is determined by reference to one or more populations of patients which either benefited, or did not benefit from similar administrations of T cells. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of predicting whether a cancer patient is likely to benefit from administration of a population of T cells, either alone or in addition to another anti-cancer therapy, comprising the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of predicting whether a cancer patient is likely to benefit from administration of a population of tumor infiltrating lymphocytes (TILs), either alone or in addition to another anti-cancer therapy, comprising the steps of: obtaining an analytical signature of a blood-derived sample from the patient; and determining that the analytical signature is correlated or anti-correlated with a biological marker that correlates or anti-correlates with the likelihood of the patent to benefit from such administration. In some embodiments, such likelihood is determined by reference to one or more populations of patients which either benefited, or did not benefit from similar administrations of TILs.

In one embodiment, the invention provides a method of predicting whether a cancer patient is likely to benefit from administration of a population of tumor infiltrating lymphocytes (TILs), either alone or in addition to another anti-cancer therapy, comprising the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In some embodiments, the analytical signature is obtained by a mass spectrometry method, an electrophoresis method, or a chromatography method. In some embodiments, the analytical signature is obtained by a mass spectrometry method, and comprises integrated intensity values of selected mass spectral features over predefined m/z ranges. In some embodiments, the mass spectral m/z ranges are one or more ranges listed in Table 16. In some embodiments, the mass spectral features are one or more features listed in Table 22. In some embodiments, mass-spectrometry is conducted in positive ion mode.

In one embodiment, the invention provides a method of treating cancer in a patient having a cancer-related tumor, wherein the patient is likely to benefit from administration of T cells comparative to a group of other cancer patients that have been administered T cells, comprising the steps of: contacting a first population of T cells with a first cell culture medium; and performing an initial expansion of the first population of T cells in the first cell culture medium to obtain a second population of T cells. In one embodiment, the invention provides a method of treating cancer in a patient having a cancer-related tumor, wherein the patient is likely to benefit from administration of T cells comparative to a group of other cancer patients that have been administered T cells, comprising the steps of: obtaining a population of T cells; contacting the population with a first cell culture medium; and performing an initial expansion of the first population of T cells in the first cell culture medium to obtain a second population of T cells. In some embodiments, the method comprises receiving a first population of T cells from the patient. In some embodiments, the second population of T cells is at least 5-fold greater in number than the first population of T cells. In some embodiments, the first cell culture medium comprises IL-2. In some embodiments, the method further comprises performing a rapid expansion of the second population of T cells in a second cell culture medium to obtain a third population of T cells. In some embodiments, the third population of TILs is at least 50-fold greater in number than the second population of T cells after 7 days from the start of the rapid expansion. In some embodiments, the second cell culture medium comprises IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further comprises harvesting the third population of TILs; and administering a therapeutically effective portion of the third population of T cells to the patient. In some embodiments, the likelihood of beneficial administration of T cells is determined by a serum based analytical assay comprising: obtaining an analytical signature of a blood-derived sample from the patient; comparing the analytical signature with a training set of analytical signatures of samples from a group of other cancer patients that have been administered T cells, wherein the analytical signatures are class-labeled good, intermediate, bad, late, early, plus (+), or minus (−); and classifying the patient sample with the class label good, late, or plus (+). In some embodiments, subgroups of the other cancer patients that have been administered T cells achieved a complete response, a partial response, no response, a stable disease state, or a progressive disease state. In some embodiments, subgroups of the other cancer patients that have been administered T cells had no disease progression for about one year, about two years, about three years, about four years, about five years, or more than five years. In some embodiments, subgroups of the other cancer patients that have been administered T cells achieved progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the class label good, late, or plus (+), is associated with progression free survival of about 24 months, about 30 months, about 36 months, about 42 months, about 48 months, about 54 months, about 60 months, up to 60 months, or more than 60 months. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient is likely to benefit from administration of TILs comparative to a group of other cancer patients that have been administered TILs, comprising the steps of: obtaining from the patient a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In one embodiment, the invention provides a method of treating cancer in a patient having a cancer-related tumor, wherein the patient is likely to benefit from administration of TILs comparative to a group of other cancer patients that have been administered TILs, comprising the steps of: receiving a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In some embodiments, the likelihood of beneficial administration of TILs is determined by a serum based analytical assay comprising: obtaining an analytical signature of a blood-derived sample from the patient; comparing the analytical signature with a training set of analytical signatures of samples from a group of other cancer patients that have been administered TILs, wherein the analytical signatures are class-labeled good, intermediate, bad, late, early, plus (+), or minus (−); and classifying the patient sample with the class label good, late, or plus (+). In some embodiments, subgroups of the other cancer patients that have been administered TILs achieved a complete response, a partial response, no response, a stable disease state, or a progressive disease state. In some embodiments, subgroups of the other cancer patients that have been administered TILs had no disease progression for about one year, about two years, about three years, about four years, about five years, or more than five years. In some embodiments, subgroups of the other cancer patients that have been administered TILs achieved progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the class label good, late, or plus (+), is associated with progression free survival of about 24 months, about 30 months, about 36 months, about 42 months, about 48 months, about 54 months, about 60 months, up to 60 months, or more than 60 months.

In some embodiments, the analytical signature is obtained by a mass spectrometry method, an electrophoresis method, or a chromatography method. In some embodiments, the analytical signature is obtained by a mass spectrometry method, and the analytical signature comprises integrated intensity values of selected mass spectral features over predefined m/z ranges. In some embodiments, the mass spectral features are correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

In one embodiment, the invention provides a method of treating cancer in a patient having a cancer-related tumor, wherein the patient is likely to benefit from administration of T cells, comprising the steps of: obtaining a population of T cells; contacting the population with a first cell culture medium; and performing an initial expansion of the first population of T cells in the first cell culture medium to obtain a second population of T cells. In one embodiment, the invention provides a method of treating cancer in a patient having a cancer-related tumor, wherein the patient is likely to benefit from administration of T cells, comprising the steps of: receiving a population of T cells; contacting the population with a first cell culture medium; and performing an initial expansion of the first population of T cells in the first cell culture medium to obtain a second population of T cells. In some embodiments, the second population of T cells is at least 5-fold greater in number than the first population of T cells. In some embodiments, the first cell culture medium comprises IL-2. In some embodiments, the method further comprises performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium comprises IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further comprises harvesting the third population of T cells and administering a therapeutically effective portion of the third population of T cells to the patient. In some embodiments, the likelihood of beneficial administration of T cells is determined by a serum based analytical method, comprising the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the analytical signature is obtained by a mass spectrometry method, an electrophoresis method, or a chromatography method. In some embodiments, the analytical signature is obtained by a mass spectrometry method, and the analytical signature comprises integrated intensity values of selected mass spectral features over predefined m/z ranges. In some embodiments, the mass spectral m/z ranges are one or more ranges listed in Table 16. In some embodiments, the mass spectral features are one or more features listed in Table 22. In some embodiments, mass-spectrometry is conducted in positive ion mode. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient is likely to benefit from administration of TILs, comprising the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In one embodiment, the invention provides a method of treating cancer in a patient having a cancer-related tumor, wherein the patient is likely to benefit from administration of TILs, comprising the steps of: receiving a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In some embodiments, the likelihood of beneficial administration of TILs is determined by a serum based analytical method, comprising the steps of: obtaining an analytical signature of a blood-derived sample from the patient; and determining that the analytical signature is correlated or anti-correlated with: the complement system protein functional group, the acute inflammation protein functional group, the acute response protein functional group, or the acute phase protein functional group; or the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin. In some embodiments, the analytical signature is obtained by a mass spectrometry method, an electrophoresis method, or a chromatography method. In some embodiments, the analytical signature is obtained by a mass spectrometry method, and the analytical signature comprises integrated intensity values of selected mass spectral features over predefined m/z ranges. In some embodiments, the mass spectral m/z ranges are one or more ranges listed in Table 16. In some embodiments, the mass spectral features are one or more features listed in Table 22. In some embodiments, mass-spectrometry is conducted in positive ion mode.

As described herein, various methods of T cells and/or TILs expansion can be used. In some embodiments, the initial expansion is performed over a period of 21 days or less. In some embodiments, the initial expansion is performed over a period of 11 days or less. In some embodiments, the rapid expansion is performed over a period of 7 days or less. In some embodiments, the IL-2 is present at an initial concentration of between 1000 IU/mL and 6000 IU/mL in the first cell culture medium. In some embodiments, the IL-2 is present at an initial concentration of between 1000 IU/mL and 6000 IU/mL and the OKT-3 antibody is present at an initial concentration of about 30 ng/mL in the second cell culture medium. In some embodiments, the initial expansion is performed using a gas permeable container. In some embodiments, the rapid expansion is performed using a gas permeable container. In some embodiments, the first cell culture medium further comprises a cytokine selected from the group consisting of IL-4, IL-7, IL-15, IL-21, and combinations thereof. In some embodiments, the second cell culture medium further comprises a cytokine selected from the group consisting of IL-4, IL-7, IL-15, IL-21, and combinations thereof.

In one embodiment, the invention provides a method of treating cancer in a patient having a cancer-related tumor, wherein the patient is likely to benefit from administration of T cells, comprising administering to the patient a therapeutically effective population of T cells, and an additional therapeutic method, method step, or agent. In some embodiments, the methods of treatment provided here further comprise the step of treating the patient with a non-myeloablative lymphodepletion regimen prior to administering the third population of T cells to the patient. In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days. In some embodiments, the methods of treatment provided here further comprise the step of treating the patient with a high-dose IL-2 regimen starting on the day after administration of the third population of T cells to the patient. In some embodiments, the high-dose IL-2 regimen further comprises aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient is likely to benefit from administration of TILs, comprising administering to the patient a therapeutically effective population of TILs, and an additional therapeutic method, method step, or agent. In some embodiments, the methods of treatment provided here further comprise the step of treating the patient with a non-myeloablative lymphodepletion regimen prior to administering the third population of TILs to the patient. In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days. In some embodiments, the methods of treatment provided here further comprise the step of treating the patient with a high-dose IL-2 regimen starting on the day after administration of the third population of TILs to the patient. In some embodiments, the high-dose IL-2 regimen further comprises aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance.

In one embodiment, the invention provides a method of treating cancer in a patient having a cancer-related tumor, wherein the patient is likely to benefit from administration of T cells, comprising administering to the patient a therapeutically effective population of T cells. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient is likely to benefit from administration of TILs, comprising administering to the patient a therapeutically effective population of TILs. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma.

In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: obtaining a first population of T cells; contacting the population with a first cell culture medium; and performing an initial expansion of the first population of T cells in the first cell culture medium to obtain a second population of T cells. In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: receiving a first population of T cells; contacting the population with a first cell culture medium; and performing an initial expansion of the first population of T cells in the first cell culture medium to obtain a second population of T cells. In some embodiments, the second population of T cells is at least 5-fold greater in number than the first population of T cells. In some embodiments, the first cell culture medium comprises IL-2. In some embodiments, the method further comprises performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium comprises IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further comprises harvesting the third population of T cells, and administering a therapeutically effective portion of the third population of T cells to the patient. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁻/HER2⁺), triple negative breast cancer (ER⁻/PR⁻/HER2⁺), double-refractory melanoma, and uveal (ocular) melanoma. In some embodiments, the level of protein expression is increased or decreased as compared to a healthy subject. In some embodiments, the level of protein expression is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: obtaining a first population of T cells; contacting the population with a first cell culture medium; and performing an initial expansion of the first population of T cells in the first cell culture medium to obtain a second population of T cells. In one embodiment, the invention provides a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: receiving a first population of T cells; contacting the population with a first cell culture medium; and performing an initial expansion of the first population of T cells in the first cell culture medium to obtain a second population of T cells. In some embodiments, the second population of T cells is at least 5-fold greater in number than the first population of T cells. In some embodiments, the first cell culture medium comprises IL-2. In some embodiments, the method further comprises performing a rapid expansion of the second population of T cells in a second cell culture medium to obtain a third population of T cells. In some embodiments, the third population of T cells is at least 50-fold greater in number than the second population of T cells after 7 days from the start of the rapid expansion. In some embodiments, the second cell culture medium comprises IL-2, OKT-3 (anti-CD3 antibody), and irradiated allogeneic peripheral blood mononuclear cells (PBMCs). In some embodiments, the rapid expansion is performed over a period of 14 days or less. In some embodiments, the method further comprises harvesting the third population of T cells; and administering a therapeutically effective portion of the third population of T cells to the patient. In some embodiments, the different cancer patient has been previously treated with a population of T cells. In some embodiments, the other cancer patient achieved a post-treatment complete response, partial response, or a stable disease state. In some embodiments, the other cancer patient achieved had no post-treatment disease progression for about one year, about two years, about three years, about four years, about five years, or more than five years. In some embodiments, the other cancer patient achieved post-treatment progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁻/PR⁻/HER2⁺), triple negative breast cancer (ER⁻/PR⁻/HER2⁺), double-refractory melanoma, and uveal (ocular) melanoma. In some embodiments, the level of protein expression similarity is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-Hb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In one embodiment, the invention provides a method of treating cancer in a patient having a cancer-related tumor, wherein the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: receiving a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the level of protein expression is increased or decreased as compared to a healthy subject. In some embodiments, the level of protein expression is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In one embodiment, the invention provides a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: obtaining a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient, wherein the different cancer patient has been previously treated with a population of TILs. In one embodiment, the invention provides a method of treating cancer in a patient having a cancer-related tumor, wherein compared to a different cancer patient, the patient exhibits a similar level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of: receiving a tumor fragment comprising a first population of TILs; contacting the tumor fragment with a first cell culture medium; performing an initial expansion of the first population of TILs in the first cell culture medium to obtain a second population of TILs; wherein the second population of TILs is at least 5-fold greater in number than the first population of TILs; and wherein the first cell culture medium comprises IL-2; performing a rapid expansion of the second population of TILs in a second cell culture medium to obtain a third population of TILs; wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and harvesting the third population of TILs. In some embodiments, the method further comprises administering a therapeutically effective portion of the third population of TILs to the patient, wherein the different cancer patient has been previously treated with a population of TILs. In some embodiments, the other cancer patient achieved a post-treatment complete response, partial response, or a stable disease state. In some embodiments, the other cancer patient achieved had no post-treatment disease progression for about one year, about two years, about three years, about four years, about five years, or more than five years. In some embodiments, the other cancer patient achieved post-treatment progression free survival of less than 6 months, about 6 months, about 12 months, about 18 months, about 24 months, about 30 months, about 36 months, about 42 months, about 48 months, about 54 months, about 60 months, up to 60 months, or more than 60 months. In some embodiments, the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the level of protein expression similarity is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In one embodiment, the invention provides a method of treating cancer in a patient having a cancer-related tumor, wherein the patient is likely to benefit from administration of T cells, comprising administering to the patient a therapeutically effective population of T cells wherein the T cells where obtained through a method including one or more expansion steps, such as an initial expansion, and/or a rapid expansion, and including various culture mediums as described herein. In some embodiments, the initial expansion is performed over a period of 21 days or less. In some embodiments, the initial expansion is performed over a period of 11 days or less. In some embodiments, the rapid expansion is performed over a period of 7 days or less. In some embodiments, the IL-2 is present at an initial concentration of between 1000 IU/mL and 6000 IU/mL in the first cell culture medium. In some embodiments, the IL-2 is present at an initial concentration of between 1000 IU/mL and 6000 IU/mL and the OKT-3 antibody is present at an initial concentration of about 30 ng/mL in the second cell culture medium. In some embodiments, the initial expansion is performed using a gas permeable container. In some embodiments, the rapid expansion is performed using a gas permeable container. In some embodiments, the first cell culture medium further comprises a cytokine selected from the group consisting of IL-4, IL-7, IL-15, IL-21, and combinations thereof. In some embodiments, the second cell culture medium further comprises a cytokine selected from the group consisting of IL-4, IL-7, IL-15, IL-21, and combinations thereof. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, wherein the patient is likely to benefit from administration of TILs, comprising administering to the patient a therapeutically effective population of TILs wherein the TILs where obtained through a method including one or more expansion steps, such as an initial expansion, and/or a rapid expansion, and including various culture mediums as described herein. In some embodiments, the initial expansion is performed over a period of 21 days or less. In some embodiments, the initial expansion is performed over a period of 11 days or less. In some embodiments, the rapid expansion is performed over a period of 7 days or less. In some embodiments, the IL-2 is present at an initial concentration of between 1000 IU/mL and 6000 IU/mL in the first cell culture medium. In some embodiments, the IL-2 is present at an initial concentration of between 1000 IU/mL and 6000 IU/mL and the OKT-3 antibody is present at an initial concentration of about 30 ng/mL in the second cell culture medium. In some embodiments, the initial expansion is performed using a gas permeable container. In some embodiments, the rapid expansion is performed using a gas permeable container. In some embodiments, the first cell culture medium further comprises a cytokine selected from the group consisting of IL-4, IL-7, IL-15, IL-21, and combinations thereof. In some embodiments, the second cell culture medium further comprises a cytokine selected from the group consisting of IL-4, IL-7, IL-15, IL-21, and combinations thereof.

In one embodiment, the invention provides a method of treating cancer in a patient having a cancer-related tumor, comprising administering to the patient a population of T cells, the method further comprising the step of treating the patient with a non-myeloablative lymphodepletion regimen prior to administering the population of T cells to the patient. In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days. In some embodiments, the method further comprises the step of treating the patient with a high-dose IL-2 regimen starting on the day after administration of the population of T cells to the patient. In some embodiments, the high-dose IL-2 regimen further comprises aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In one embodiment, the invention provides a method of treating cancer in a patient having a cancer-related tumor, comprising administering to the patient a population of TILs, the method further comprising the step of treating the patient with a non-myeloablative lymphodepletion regimen prior to administering the population of TILs to the patient. In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days. In some embodiments, the method further comprises the step of treating the patient with a high-dose IL-2 regimen starting on the day after administration of the population of TILs to the patient. In some embodiments, the high-dose IL-2 regimen further comprises aldesleukin, or a biosimilar or variant thereof. In some embodiments, aldesleukin, or a biosimilar or variant thereof, is administered at a dose of 600,000 or 720,000 IU/kg, as a 15-minute bolus intravenous infusion every eight hours until tolerance.

Methods of Expanding T Cells and/or Tumor Infiltrating Lymphocytes

In an embodiment, the invention provides a process for expanding a population of T cells including a pre-rapid expansion (pre-REP) process and a rapid expansion process (REP), wherein the cell culture medium used for expansion comprises IL-2 at a concentration selected from the group consisting of between 100 IU/mL and 10,000 IU/mL, between 200 IU/mL and 5,000 IU/mL, between 300 IU/mL and 4,800 IU/mL, between 400 IU/mL and 4,600 IU/mL, between 500 IU/mL and 4,400 IU/mL, between 600 IU/mL and 4,200 IU/mL, between 700 IU/mL and 4,000 IU/mL, between 800 IU/mL and 3,800 IU/mL, between 900 IU/mL and 3,600 IU/mL, between 1,000 IU/mL and 3,400 IU/mL, between 1,100 IU/mL and 3,200 IU/mL, between 1,200 IU/mL and 3,000 IU/mL, between 1,300 IU/mL and 2,800 IU/mL, between 1,400 IU/mL and 2,600 IU/mL, between 1,500 IU/mL and 2,400 IU/mL, between 1,600 IU/mL and 2,200 IU/mL, between 1,700 IU/mL and 2,000 IU/mL, between 5,500 IU/mL and 9,500 IU/mL, between 6,000 IU/mL and 9,000 IU/mL, between 6500 IU/mL and 8,500 IU/mL, between 7,000 IU/mL and 8,000 IU/mL, and between 7,500 IU/mL and 8,000 IU/mL. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a process for expanding a population of TILs including a pre-rapid expansion (pre-REP) process and a rapid expansion process (REP), wherein the cell culture medium used for expansion comprises IL-2 at a concentration selected from the group consisting of between 100 IU/mL and 10,000 IU/mL, between 200 IU/mL and 5,000 IU/mL, between 300 IU/mL and 4,800 IU/mL, between 400 IU/mL and 4,600 IU/mL, between 500 IU/mL and 4,400 IU/mL, between 600 IU/mL and 4,200 IU/mL, between 700 IU/mL and 4,000 IU/mL, between 800 IU/mL and 3,800 IU/mL, between 900 IU/mL and 3,600 IU/mL, between 1,000 IU/mL and 3,400 IU/mL, between 1,100 IU/mL and 3,200 IU/mL, between 1,200 IU/mL and 3,000 IU/mL, between 1,300 IU/mL and 2,800 IU/mL, between 1,400 IU/mL and 2,600 IU/mL, between 1,500 IU/mL and 2,400 IU/mL, between 1,600 IU/mL and 2,200 IU/mL, between 1,700 IU/mL and 2,000 IU/mL, between 5,500 IU/mL and 9,500 IU/mL, between 6,000 IU/mL and 9,000 IU/mL, between 6500 IU/mL and 8,500 IU/mL, between 7,000 IU/mL and 8,000 IU/mL, and between 7,500 IU/mL and 8,000 IU/mL.

In an embodiment, the invention provides a process for expanding a population of T cells including a pre-rapid expansion (pre-REP) process and a rapid expansion process (REP), wherein the cell culture medium used for expansion comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a process for expanding a population of TILs including a pre-rapid expansion (pre-REP) process and a rapid expansion process (REP), wherein the cell culture medium used for expansion comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In an embodiment, the invention provides a pre-REP process for expanding a population of T cells, the process comprising the steps of contacting the population of T cells with a cell culture medium comprising IL-2 at an initial concentration of between 1000 IU/mL and 6000 IU/mL, wherein the population of T cells comprises T cells with a phenotype selected from the group consisting CD8⁺CD28⁺, CD8⁺CD27⁺, CD8⁺CD27⁺CD28⁺, CCR7⁺, and combinations thereof. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a pre-REP process for expanding a population of TILs, the process comprising the steps of contacting the population of TILs with a cell culture medium comprising IL-2 at an initial concentration of between 1000 IU/mL and 6000 IU/mL, wherein the population of TILs comprises T cells with a phenotype selected from the group consisting CD8⁺CD28⁺, CD8⁺CD27⁺, CD8⁺CD27⁺CD28⁺, CCR7⁺, and combinations thereof.

In an embodiment, the invention provides a pre-REP process of expanding a population of T cells, the process comprising the steps of contacting the population of T cells with a cell culture medium comprising IL-2 at an initial concentration of between 1000 IU/mL and 6000 IU/mL, wherein the population of T cells is expanded over a period of time selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 25 days, 30 days, 35 days, and 40 days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a pre-REP process of expanding a population of TILs, the process comprising the steps of contacting the population of TILs with a cell culture medium comprising IL-2 at an initial concentration of between 1000 IU/mL and 6000 IU/mL, wherein the population of TILs is expanded over a period of time selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 25 days, 30 days, 35 days, and 40 days.

In an embodiment, the invention provides a pre-REP process of expanding a population of T cells, the process comprising the steps of contacting the population of T cells with a cell culture medium, wherein the cell culture medium comprises IL-2 at an initial concentration of between 1000 IU/mL and 6000 IU/mL, wherein the population of TILs is expanded over a period of time selected from the group consisting of less than 1 day, less than 2 days, less than 3 days, less than 4 days, less than 5 days, less than 6 days, less than 7 days, less than 8 days, less than 9 days, less than 10 days, less than 11 days, less than 12 days, less than 13 days, less than 14 days, less than 15 days, less than 16 days, less than 17 days, less than 18 days, less than 19 days, less than 20 days, less than 21 days, less than 25 days, less than 30 days, less than 35 days, and less than 40 days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a pre-REP process of expanding a population of TILs, the process comprising the steps of contacting the population of TILs with a cell culture medium, wherein the cell culture medium comprises IL-2 at an initial concentration of between 1000 IU/mL and 6000 IU/mL, wherein the population of TILs is expanded over a period of time selected from the group consisting of less than 1 day, less than 2 days, less than 3 days, less than 4 days, less than 5 days, less than 6 days, less than 7 days, less than 8 days, less than 9 days, less than 10 days, less than 11 days, less than 12 days, less than 13 days, less than 14 days, less than 15 days, less than 16 days, less than 17 days, less than 18 days, less than 19 days, less than 20 days, less than 21 days, less than 25 days, less than 30 days, less than 35 days, and less than 40 days.

In an embodiment, the invention provides a method of expanding a population of T cells, the method comprising the steps of contacting the population of T cells with a cell culture medium, wherein the cell culture medium comprises IL-2 at an initial concentration of between 1000 IU/mL and 6000 IU/mL and OKT-3 antibody at an initial concentration of about 30 ng/mL. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a method of expanding a population of TILs, the method comprising the steps of contacting the population of TILs with a cell culture medium, wherein the cell culture medium comprises IL-2 at an initial concentration of between 1000 IU/mL and 6000 IU/mL and OKT-3 antibody at an initial concentration of about 30 ng/mL.

In an embodiment, the invention provides a REP process for expanding a population of T cells, the process comprising the steps of contacting the population of T cells with a cell culture medium, wherein the cell culture medium comprises IL-2 at an initial concentration of about 6000 IU/mL and OKT-3 antibody at an initial concentration of about 30 ng/mL. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process for expanding a population of TILs, the process comprising the steps of contacting the population of TILs with a cell culture medium, wherein the cell culture medium comprises IL-2 at an initial concentration of about 6000 IU/mL and OKT-3 antibody at an initial concentration of about 30 ng/mL.

In an embodiment, the invention provides a REP process of expanding a population of T cells, the process comprising the steps of contacting the population of T cells with a cell culture medium, wherein the population of T cells expands by at least 50-fold over a period of 7 days in the cell culture medium. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process of expanding a population of tumor infiltrating lymphocytes (TILs), the process comprising the steps of contacting the population of TILs with a cell culture medium, wherein the population of TILs expands by at least 50-fold over a period of 7 days in the cell culture medium.

In an embodiment, the invention provides a REP process of expanding a population of T cells, the process comprising the steps of contacting the population of T cells with a cell culture medium, wherein the population of T cells expands by at least 50-fold over a period of 7 days in the cell culture medium, and wherein the expansion is performed using a gas permeable container. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process of expanding a population of tumor infiltrating lymphocytes (TILs), the process comprising the steps of contacting the population of TILs with a cell culture medium, wherein the population of TILs expands by at least 50-fold over a period of 7 days in the cell culture medium, and wherein the expansion is performed using a gas permeable container.

In an embodiment, the invention provides a REP process of expanding a population of T cells, the process comprising the steps of contacting the population of T cells with a cell culture medium, wherein the population of T cells expands by at least 50-fold over a period of 7 days in the cell culture medium, and wherein the expansion is performed using a gas permeable container, wherein the gas permeable container is a gas permeable bag or a gas permeable flask. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process of expanding a population of tumor infiltrating lymphocytes (TILs), the process comprising the steps of contacting the population of TILs with a cell culture medium, wherein the population of TILs expands by at least 50-fold over a period of 7 days in the cell culture medium, and wherein the expansion is performed using a gas permeable container, wherein the gas permeable container is a gas permeable bag or a gas permeable flask.

In an embodiment, the invention provides a REP process of expanding a population of T cells, the process comprising the steps of contacting the population of T cells with a cell culture medium, wherein the cell culture medium comprises IL-2 at an initial concentration of between 1000 IU/mL and 6000 IU/mL and OKT-3 antibody at an initial concentration of about 30 ng/mL, wherein the population of T cells is rapidly expanded over a period of time selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 25 days, 30 days, 35 days, and 40 days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process of expanding a population of TILs, the process comprising the steps of contacting the population of TILs with a cell culture medium, wherein the cell culture medium comprises IL-2 at an initial concentration of between 1000 IU/mL and 6000 IU/mL and OKT-3 antibody at an initial concentration of about 30 ng/mL, wherein the population of TILs is rapidly expanded over a period of time selected from the group consisting of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 25 days, 30 days, 35 days, and 40 days.

In an embodiment, the invention provides a REP process of expanding a population of T cells, the process comprising the steps of contacting the population of T cells with a cell culture medium, wherein the cell culture medium comprises IL-2 at an initial concentration of between 1000 IU/mL and 6000 IU/mL and OKT-3 antibody at an initial concentration of about 30 ng/mL, wherein the population of T cells is rapidly expanded over a period of time selected from the group consisting of less than 1 day, less than 2 days, less than 3 days, less than 4 days, less than 5 days, less than 6 days, less than 7 days, less than 8 days, less than 9 days, less than 10 days, less than 11 days, less than 12 days, less than 13 days, less than 14 days, less than 15 days, less than 16 days, less than 17 days, less than 18 days, less than 19 days, less than 20 days, less than 21 days, less than 25 days, less than 30 days, less than 35 days, and less than 40 days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a REP process of expanding a population of TILs, the process comprising the steps of contacting the population of TILs with a cell culture medium, wherein the cell culture medium comprises IL-2 at an initial concentration of between 1000 IU/mL and 6000 IU/mL and OKT-3 antibody at an initial concentration of about 30 ng/mL, wherein the population of TILs is rapidly expanded over a period of time selected from the group consisting of less than 1 day, less than 2 days, less than 3 days, less than 4 days, less than 5 days, less than 6 days, less than 7 days, less than 8 days, less than 9 days, less than 10 days, less than 11 days, less than 12 days, less than 13 days, less than 14 days, less than 15 days, less than 16 days, less than 17 days, less than 18 days, less than 19 days, less than 20 days, less than 21 days, less than 25 days, less than 30 days, less than 35 days, and less than 40 days.

In an embodiment, REP can be performed in a gas permeable container by any suitable method. For example, T cells or TILs can be rapidly expanded using non-specific T cell receptor stimulation in the presence of interleukin-2 (IL-2) or interleukin-15 (IL-15). The non-specific T cell receptor stimulus can include, for example, about 30 ng/mL of OKT-3, a monoclonal anti-CD3 antibody (commercially available from Ortho-McNeil, Raritan, N.J. or Miltenyi Biotech, Auburn, Calif.). T cells or TILs can be rapidly expanded by further stimulation of the T cells or TILs in vitro with one or more antigens, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. T cells or TILs may also be rapidly expanded by re-stimulation with the same antigen(s) of the cancer pulsed onto HLA-A2-expressing antigen-presenting cells. Alternatively, the T cells or TILs can be further re-stimulated with, e.g., example, irradiated, autologous lymphocytes or with irradiated HLA-A2⁺ allogeneic lymphocytes and IL-2. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, a method for expanding T cells or TILs may include using about 5000 mL to about 25000 mL of cell culture medium, about 5000 mL to about 10000 mL of cell culture medium, or about 5800 mL to about 8700 mL of cell culture medium. In an embodiment, a method for expanding T cells or TILs may include using about 1000 mL to about 2000 mL of cell medium, about 2000 mL to about 3000 mL of cell culture medium, about 3000 mL to about 4000 mL of cell culture medium, about 4000 mL to about 5000 mL of cell culture medium, about 5000 mL to about 6000 mL of cell culture medium, about 6000 mL to about 7000 mL of cell culture medium, about 7000 mL to about 8000 mL of cell culture medium, about 8000 mL to about 9000 mL of cell culture medium, about 9000 mL to about 10000 mL of cell culture medium, about 10000 mL to about 15000 mL of cell culture medium, about 15000 mL to about 20000 mL of cell culture medium, or about 20000 mL to about 25000 mL of cell culture medium. In an embodiment, expanding the number of T cells or TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad Calif.). In this regard, the inventive methods advantageously reduce the amount of medium and the number of types of medium required to expand the number of T cells or TILs. In an embodiment, expanding the number of T cells or TILs may comprise feeding the cells no more frequently than every third or fourth day. Expanding the number of cells in a gas permeable container simplifies the procedures necessary to expand the number of cells by reducing the feeding frequency necessary to expand the cells. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the rapid expansion is performed using a gas permeable container. Such embodiments allow for cell populations to expand from about 5×10⁵ cells/cm² to between 10×10⁶ and 30×10⁶ cells/cm². In an embodiment, this expansion occurs without feeding. In an embodiment, this expansion occurs without feeding so long as medium resides at a height of about 10 cm in a gas-permeable flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739 A1, International Patent Application Publication No. WO 2014/210036 A1, U.S. Patent Application Publication No. US 2013/0115617 A1, International Publication No. WO 2013/188427 A1, U.S. Patent Application Publication No. US 2011/0136228 A1, U.S. Pat. No. 8,809,050, International Patent Application Publication No. WO 2011/072088 A2, U.S. Patent Application Publication No. US 2016/0208216 A1, U.S. Patent Application Publication No. US 2012/0244133 A1, International Patent Application Publication No. WO 2012/129201 A1, U.S. Patent Application Publication No. US 2013/0102075 A1, U.S. Pat. No. 8,956,860, International Patent Application Publication No. WO 2013/173835 A1, and U.S. Patent Application Publication No. US 2015/0175966 A1, the disclosures of which are incorporated herein by reference. Such processes are also described in Jin, et al., J. Immunotherapy 2012, 35, 283-292, the disclosure of which is incorporated by reference herein.

In an embodiment, the gas permeable container is a G-Rex 10 flask (Wilson Wolf Manufacturing Corporation, New Brighton, Minn., USA). In an embodiment, the gas permeable container includes a 10 cm² gas permeable culture surface. In an embodiment, the gas permeable container includes a 40 mL cell culture medium capacity. In an embodiment, the gas permeable container provides 100 to 300 million T cells or TILs after 2 medium exchanges. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the gas permeable container is a G-Rex 100 flask (Wilson Wolf Manufacturing Corporation, New Brighton, Minn., USA). In an embodiment, the gas permeable container includes a 100 cm² gas permeable culture surface. In an embodiment, the gas permeable container includes a 450 mL cell culture medium capacity. In an embodiment, the gas permeable container provides 1 to 3 billion T cells or TILs after 2 medium exchanges. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the gas permeable container is a G-Rex 100M flask (Wilson Wolf Manufacturing Corporation, New Brighton, Minn., USA). In an embodiment, the gas permeable container includes a 100 cm² gas permeable culture surface. In an embodiment, the gas permeable container includes a 1000 mL cell culture medium capacity. In an embodiment, the gas permeable container provides 1 to 3 billion T cells or TILs without medium exchange. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the gas permeable container is a G-Rex 100 L flask (Wilson Wolf Manufacturing Corporation, New Brighton, Minn., USA). In an embodiment, the gas permeable container includes a 100 cm² gas permeable culture surface. In an embodiment, the gas permeable container includes a 2000 mL cell culture medium capacity. In an embodiment, the gas permeable container provides 1 to 3 billion T cells or TILs without medium exchange. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the gas permeable container is a G-Rex 24 well plate (Wilson Wolf Manufacturing Corporation, New Brighton, Minn., USA). In an embodiment, the gas permeable container includes a plate with wells, wherein each well includes a 2 cm² gas permeable culture surface. In an embodiment, the gas permeable container includes a plate with wells, wherein each well includes an 8 mL cell culture medium capacity. In an embodiment, the gas permeable container provides 20 to 60 million cells per well after 2 medium exchanges.

In an embodiment, the gas permeable container is a G-Rex 6 well plate (Wilson Wolf Manufacturing Corporation, New Brighton, Minn., USA). In an embodiment, the gas permeable container includes a plate with wells, wherein each well includes a 10 cm² gas permeable culture surface. In an embodiment, the gas permeable container includes a plate with wells, wherein each well includes a 40 mL cell culture medium capacity. In an embodiment, the gas permeable container provides 100 to 300 million cells per well after 2 medium exchanges.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME).

In an embodiment, the duration of the method comprising obtaining a tumor tissue sample from the mammal; culturing the tumor tissue sample in a first gas permeable container containing cell medium therein; obtaining T cells from the tumor tissue sample; expanding the number of T cells in a second gas permeable container containing cell medium for a duration of about 14 to about 42 days, e.g., about 28 days. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the duration of the method comprising obtaining a tumor tissue sample from the mammal; culturing the tumor tissue sample in a first gas permeable container containing cell medium therein; obtaining TILs from the tumor tissue sample; expanding the number of TILs in a second gas permeable container containing cell medium for a duration of about 14 to about 42 days, e.g., about 28 days.

In an embodiment, the cell culture medium comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT-3 antibody. In a preferred embodiment, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody.

In an embodiment, T cells or TILs are expanded in gas-permeable containers. Gas-permeable containers have been used to expand TILs using PBMCs using methods, compositions, and devices known in the art, including those described in U.S. Patent Application Publication No. U.S. Patent Application Publication No. 2005/0106717 A1, the disclosures of which are incorporated herein by reference. In an embodiment, T cells or TILs are expanded in gas-permeable bags. In an embodiment, T cells or TILs are expanded using a cell expansion system that expands T cells or TILs in gas permeable bags, such as the Xuri Cell Expansion System W25 (GE Healthcare). In an embodiment, T cells or TILs are expanded using a cell expansion system that expands T cells or TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, about 20 L, about 25 L, and about 30 L. In an embodiment, the cell expansion system includes a gas permeable cell bag with a volume range selected from the group consisting of between 50 and 150 mL, between 150 and 250 mL, between 250 and 350 mL, between 350 and 450 mL, between 450 and 550 mL, between 550 and 650 mL, between 650 and 750 mL, between 750 and 850 mL, between 850 and 950 mL, and between 950 and 1050 mL. In an embodiment, the cell expansion system includes a gas permeable cell bag with a volume range selected from the group consisting of between 1 L and 2 L, between 2 L and 3 L, between 3 L and 4 L, between 4 L and 5 L, between 5 L and 6 L, between 6 L and 7 L, between 7 L and 8 L, between 8 L and 9 L, between 9 L and 10 L, between 10 L and 11 L, between 11 L and 12 L, between 12 L and 13 L, between 13 L and 14 L, between 14 L and 15 L, between 15 L and 16 L, between 16 L and 17 L, between 17 L and 18 L, between 18 L and 19 L, and between 19 L and 20 L. In an embodiment, the cell expansion system includes a gas permeable cell bag with a volume range selected from the group consisting of between 0.5 L and 5 L, between 5 L and 10 L, between 10 L and 15 L, between 15 L and 20 L, between 20 L and 25 L, and between 25 L and 30 L. In an embodiment, the cell expansion system utilizes a rocking time of about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, and about 28 days. In an embodiment, the cell expansion system utilizes a rocking time of between 30 minutes and 1 hour, between 1 hour and 12 hours, between 12 hours and 1 day, between 1 day and 7 days, between 7 days and 14 days, between 14 days and 21 days, and between 21 days and 28 days. In an embodiment, the cell expansion system utilizes a rocking rate of about 2 rocks/minute, about 5 rocks/minute, about 10 rocks/minute, about 20 rocks/minute, about 30 rocks/minute, and about 40 rocks/minute. In an embodiment, the cell expansion system utilizes a rocking rate of between 2 rocks/minute and 5 rocks/minute, 5 rocks/minute and 10 rocks/minute, 10 rocks/minute and 20 rocks/minute, 20 rocks/minute and 30 rocks/minute, and 30 rocks/minute and 40 rocks/minute. In an embodiment, the cell expansion system utilizes a rocking angle of about 2°, about 3°, about 4°, about 5°, about 6°, about 7°, about 8°, about 9°, about 10°, about 11°, and about 12°. In an embodiment, the cell expansion system utilizes a rocking angle of between 2° and 3°, between 3° and 4°, between 4° and 5°, between 5° and 6°, between 6° and 7°, between 7° and 8°, between 8° and 9°, between 9° and 10°, between 10° and 11°, and between 11° and 12°. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, a method of expanding T cells or TILs further comprises a step wherein T cells or TILs are selected for superior tumor reactivity. Any selection method known in the art may be used. For example, the methods described in U.S. Patent Application Publication No. 2016/0010058 A1, the disclosures of which are incorporated herein by reference, may be used for selection of T cells or TILs for superior tumor reactivity. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, the invention provides a method of expanding a population of TILs, the method comprising the steps as described in Jin, et al., J. Immunotherapy 2012, 35, 283-292, the disclosure of which is incorporated by reference herein. For example, the tumor or portion thereof may be placed in enzyme media and mechanically dissociated for approximately 1 minute. The mixture may then be incubated for 30 minutes at 37° C. in 5% CO₂ and then mechanically disrupted again for approximately 1 minute. After incubation for 30 minutes at 37° C. in 5% CO₂, the tumor or portion thereof may be mechanically disrupted a third time for approximately 1 minute. If after the third mechanical disruption, large pieces of tissue are present, 1 or 2 additional mechanical dissociations may be applied to the sample, with or without 30 additional minutes of incubation at 37° C. in 5% CO₂. At the end of the final incubation, if the cell suspension contains a large number of red blood cells or dead cells, a density gradient separation using Ficoll may be performed to remove these cells. TIL cultures were initiated in 24-well plates (Costar 24-well cell culture cluster, flat bottom; Corning Incorporated, Corning, N.Y.), each well may be seeded with 1×10⁶ tumor digest cells or one tumor fragment approximately 1 to 8 mm³ in size in 2 mL of complete medium (CM) with IL-2 (6000 IU/mL; Chiron Corp., Emeryville, Calif.). CM comprises Roswell Park Memorial Institute (RPMI) 1640 buffer with GlutaMAX, supplemented with 10% human AB serum, 25 mM Hepes, and 10 mg/mL gentamicin. Cultures may be initiated in gas-permeable flasks with a 40 mL capacity and a 10 cm² gas-permeable silicon bottom (G-Rex 10; Wilson Wolf Manufacturing, New Brighton, each flask may be loaded with 10-40×10⁶ viable tumor digest cells or 5-30 tumor fragments in 10-40 mL of CM with IL-2. G-Rex 10 and 24-well plates may be incubated in a humidified incubator at 37° C. in 5% CO₂ and 5 days after culture initiation, half the media may be removed and replaced with fresh CM and IL-2 and after day 5, half the media may be changed every 2-3 days. Rapid expansion protocol (REP) of TILs may be performed using T-175 flasks and gas-permeable bags or gas-permeable G-Rex flasks, as described elsewhere herein. For REP in T-175 flasks, 1×10⁶ TILs may be suspended in 150 mL of media in each flask. The TIL may be cultured in a 1 to 1 mixture of CM and AIM-V medium (50/50 medium), supplemented with 3000 IU/mL of IL-2 and 30 ng/mL of anti-CD3 antibody (OKT-3). The T-175 flasks may be incubated at 37° C. in 5% CO₂. Half the media may be changed on day 5 using 50/50 medium with 3000 IU/mL of IL-2. On day 7, cells from 2 T-175 flasks may be combined in a 3 L bag and 300 mL of AIM-V with 5% human AB serum and 3000 IU/mL of IL-2 may be added to the 300 mL of TIL suspension. The number of cells in each bag may be counted every day or two days, and fresh media may be added to keep the cell count between 0.5 and 2.0×10⁶ cells/mL. For REP in 500 mL capacity flasks with 100 cm² gas-permeable silicon bottoms (e.g., G-Rex 100, Wilson Wolf Manufacturing, as described elsewhere herein), 5×10⁶ or 10×10⁶ TILs may be cultured in 400 mL of 50/50 medium, supplemented with 3000 IU/mL of IL-2 and 30 ng/mL of anti-CD3 antibody (OKT-3). The G-Rex100 flasks may be incubated at 37° C. in 5% CO₂. On day five, 250 mL of supernatant may be removed and placed into centrifuge bottles and centrifuged at 1500 rpm (491 g) for 10 minutes. The obtained TIL pellets may be resuspended with 150 mL of fresh 50/50 medium with 3000 IU/mL of IL-2 and added back to the G-Rex 100 flasks. When TIL are expanded serially in G-Rex 100 flasks, on day seven the TIL in each G-Rex100 are suspended in the 300 mL of media present in each flask and the cell suspension may be divided into three 100 mL aliquots that may be used to seed 3 G-Rex100 flasks. About 150 mL of AIM-V with 5% human AB serum and 3000 IU/mL of IL-2 may then be added to each flask. G-Rex 100 flasks may then be incubated at 37° C. in 5% CO₂, and after four days, 150 mL of AIM-V with 3000 IU/mL of IL-2 may be added to each G-Rex 100 flask. After this, the REP may be completed by harvesting cells on day 14 of culture. In some embodiments, the method can be used to expand any T cell. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, a method of expanding or treating a cancer includes a step wherein T cells or TILs are obtained from a patient tumor sample. A patient tumor sample may be obtained using methods known in the art. For example, T cells or TILs may be cultured from enzymatic tumor digests and tumor fragments (about 1 to about 8 mm³ in size) from sharp dissection. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding T cells or TILs or methods treating a cancer. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, a rapid expansion process for T cells or TILs may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks, commercially available from Wilson Wolf Manufacturing Corporation, New Brighton, Minn., USA). For T cells or TIL rapid expansion in T-175 flasks, 1×10⁶ TILs suspended in 150 mL of media may be added to each T-175 flask. The T cells or TILs may be cultured in a 1 to 1 mixture of CM and AIM-V medium, supplemented with 3000 IU (international units) per mL of IL-2 and 30 ng per ml of anti-CD3 antibody (e.g., OKT-3). The T-175 flasks may be incubated at 37° C. in 5% CO₂. Half the media may be exchanged on day 5 using 50/50 medium with 3000 IU per mL of IL-2. On day 7 cells from two T-175 flasks may be combined in a 3 L bag and 300 mL of AIM V with 5% human AB serum and 3000 IU per mL of IL-2 was added to the 300 ml of TIL suspension. The number of cells in each bag was counted every day or two and fresh media was added to keep the cell count between 0.5 and 2.0×10⁶ cells/mL. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, for T cells or TIL rapid expansions in 500 mL capacity gas permeable flasks with 100² cm gas-permeable silicon bottoms (G-Rex 100, commercially available from Wilson Wolf Manufacturing Corporation, New Brighton, Minn., USA), 5×10⁶ or 10×10⁶ TIL may be cultured in 400 mL of 50/50 medium, supplemented with 5% human AB serum, 3000 IU per mL of IL-2 and 30 ng per mL of anti-CD3 (OKT-3). The G-Rex 100 flasks may be incubated at 37° C. in 5% CO₂. On day 5, 250 mL of supernatant may be removed and placed into centrifuge bottles and centrifuged at 1500 rpm (revolutions per minute; 491×g) for 10 minutes. The T cells or TIL pellets may be re-suspended with 150 mL of fresh medium with 5% human AB serum, 3000 IU per mL of IL-2, and added back to the original G-Rex 100 flasks. When T cells or TILs are expanded serially in G-Rex 100 flasks, on day 7 the T cells or TILs in each G-Rex 100 flask may be suspended in the 300 mL of media present in each flask and the cell suspension may be divided into 3100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-Rex 100 flask. The cells may be harvested on day 14 of culture. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In an embodiment, T cells or TILs may be prepared as follows. 2 mm³ tumor fragments are cultured in complete media (CM) comprised of AIM-V medium (Invitrogen Life Technologies, Carlsbad, Calif.) supplemented with 2 mM glutamine (Mediatech, Inc. Manassas, Va.), 100 U/mL penicillin (Invitrogen Life Technologies), 100 μg/mL streptomycin (Invitrogen Life Technologies), 5% heat-inactivated human AB serum (Valley Biomedical, Inc. Winchester, Va.) and 600 IU/mL rhIL-2 (Chiron, Emeryville, Calif.). For enzymatic digestion of solid tumors, tumor specimens are diced into RPMI-1640, washed and centrifuged at 800 rpm for 5 minutes at 15-22° C., and resuspended in enzymatic digestion buffer (0.2 mg/mL Collagenase and 30 units/ml of DNase in RPMI-1640) followed by overnight rotation at room temperature. T cells or TILs established from fragments may be grown for 3-4 weeks in CM and expanded fresh or cryopreserved in heat-inactivated HAB serum with 10% dimethylsulfoxide (DMSO) and stored at −180° C. until the time of study. Tumor associated lymphocytes (TAL) obtained from ascites collections can be seeded at 3×10⁶ cells/well of a 24 well plate in CM. T cells or TIL growth can be inspected about every other day using a low-power inverted microscope. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the methods of the present invention described for the expansion of TILs may also be applied to the expansion of T cells. In some embodiments, the methods of the present invention described for the expansion of TILs may also be applied to the expansion of CD8⁺ T cells. In some embodiments, the methods of the present invention described for the expansion of TILs may also be applied to the expansion of CD4⁺ T cells. In some embodiments, the methods of the present invention described for the expansion of TILs may also be applied to the expansion of T cells transduced with a chimeric antigen receptor (CAR-T). In some embodiments, the methods of the present invention described for the expansion of TILs may also be applied to the expansion of T cells comprising a modified T cell receptor (TCR). The CAR-T cells may be targeted against any suitable antigen, including CD19, as described in the art, e.g., in U.S. Pat. Nos. 7,070,995; 7,446,190; 8,399,645; 8,916,381; and 9,328,156; the disclosures of which are incorporated by reference herein. The modified TCR cells may be targeted against any suitable antigen, including NY-ESO-1, TRP-1, TRP-2, tyrosinase cancer antigen, MAGE-A3, SSX-2, and VEGFR2, or antigenic portions thereof, as described in the art, e.g., in U.S. Pat. Nos. 8,367,804 and 7,569,664, the disclosures of which are incorporated by reference herein.

### Pharmaceutical Compositions, Dosages, and Dosing Regimens for TILs

In an embodiment, T cells or TILs expanded using methods of the present disclosure are administered to a patient as a pharmaceutical composition. In an embodiment, the pharmaceutical composition is a suspension of T cells or TILs in a sterile buffer. T cells or TILs expanded using methods of the present disclosure may be administered by any suitable route as known in the art. Preferably, the T cells or TILs are administered as a single intra-arterial or intravenous infusion, which preferably lasts approximately 30 to 60 minutes. Other suitable routes of administration include intraperitoneal, intrathecal, and intralymphatic administration. Any suitable dose of T cells or TILs can be administered. Preferably, from about 2.3×10¹⁰ to about 13.7×10¹⁰ T cells or TILs are administered, with an average of around 7.8×10¹⁰ T cells or TILs, particularly if the cancer is melanoma. In an embodiment, about 1.2×10¹⁰ to about 4.3×10¹⁰ of T cells or TILs are administered. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the number of the T cells or TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the T cells or TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the concentration of the T cells or TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the concentration of the T cells or TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the concentration of the T cells or TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the concentration of the T cells or TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the amount of the T cells or TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the amount of the T cells or TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

The T cells or TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the T cells or TILs may also be used if appropriate. The amounts of the pharmaceutical compositions administered using the methods herein, such as the dosages of T cells or TILs, will be dependent on the human or mammal being treated, the severity of the disorder or condition, the rate of administration, the disposition of the active pharmaceutical ingredients and the discretion of the prescribing physician. In some embodiments, T cells or TILs may be administered in a single dose. Such administration may be by injection, e.g., intravenous injection. In some embodiments, T cells or TILs may be administered in multiple doses. Dosing may be once, twice, three times, four times, five times, six times, or more than six times per year. Dosing may be once a month, once every two weeks, once a week, or once every other day. Administration of T cells or TILs may continue as long as necessary. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, an effective dosage of T cells or TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of T cells or TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, an effective dosage of T cells or TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, an effective dosage of T cells or TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

An effective amount of the T cells or TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation or direct injection into tumor, or by inhalation.

In preferred embodiments, the invention provides a pharmaceutical composition for injection containing T cells or TILs, and combinations thereof, and a pharmaceutical excipient suitable for injection, including intratumoral injection or intravenous infusion. Components and amounts of agents in the compositions are as described herein.

In some embodiments, T cells or TILs are administered in a single dose. Such administration may be by injection, e.g., intravenous injection.

In some embodiments, T cells or TILs are administered in multiple doses. In a preferred embodiment, T cells or TILs are administered in multiple doses. Dosing of TILs may be once a month, once every two weeks, once a week, or once every other day.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating T cells or TILs in the required amounts in the appropriate media with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of T cells or TILs can be effected by any method that enables delivery to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation, intraadiposally or intrathecally.

The invention also provides kits. The kits include a combination of ready-to-administer T cells or TILs, either alone or in combinations in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, T cells or TILs and another active pharmaceutical ingredient are provided as separate compositions in separate containers within the kit. In selected embodiments, T cells or TILs are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

The kits described above are preferably for use in the treatment of the diseases and conditions described herein. In a preferred embodiment, the kits are for use in the treatment of cancer. In preferred embodiments, the kits are for use in treating solid tumor cancers. In a preferred embodiment, the kits of the present invention are for use in the treatment of cancer, including any of the cancers described herein.

### Methods of Treating Cancers

The compositions and combinations of T cells or TILs (and populations thereof) can be used in a method for treating hyperproliferative disorders. In a preferred embodiment, they are for use in treating cancers. In a preferred embodiment, the invention provides a method of treating a cancer, wherein the cancer is a hematological malignancy or a solid tumor. In a preferred embodiment, the invention provides a method of treating a cancer, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In a preferred embodiment, the invention provides a method of treating a cancer, wherein the cancer is selected from the group consisting of non-small cell lung cancer (NSCLC) or triple negative breast cancer, double-refractory melanoma, and uveal (ocular) melanoma. In a preferred embodiment, the invention provides a method of treating a cancer, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma with T cells or TILs. In a preferred embodiment, the invention provides a method of treating a cancer, wherein the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁻/PR⁻/HER2⁺), triple negative breast cancer (ER⁻/PR⁻/HER2⁻), double-refractory melanoma, and uveal (ocular) melanoma with T cells or TILs. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

In some embodiments, the invention provides a method of treating a cancer with a population of tumor infiltrating lymphocytes (TILs) comprising the steps of:
(a) resecting a tumor from a patient, the tumor comprising a first population of TILs;
(b) fragmenting the tumor to obtain tumor fragments;
(c) contacting the tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less;
(f) harvesting the third population of TILs; and
(g) administering a therapeutically effective portion of the third population of TILs to a patient with the cancer;
   wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma.

In some embodiments, the invention provides a method of treating a cancer with a population of tumor infiltrating lymphocytes (TILs) comprising the steps of:
(a) receiving a tumor or tumor fragment from a patient, the tumor or tumor fragment comprising a first population of TILs;
(b) optionally fragmenting the tumor to obtain tumor fragments;
(c) contacting the tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and
(f) harvesting the third population of TILs;
   wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the method further comprises administering a therapeutically effective portion of the third population of TILs to a patient with the cancer.

In some embodiments, the invention provides a method of treating a cancer with a population of tumor infiltrating lymphocytes (TILs) comprising the steps of:
(a) resecting a tumor from a patient, the tumor comprising a first population of TILs;
(b) fragmenting the tumor to obtain tumor fragments;
(c) contacting the tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less;
(f) harvesting the third population of TILs; and
(g) administering a therapeutically effective portion of the third population of TILs to a patient with the cancer;
   wherein the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma.

In some embodiments, the invention provides a method of treating a cancer with a population of tumor infiltrating lymphocytes (TILs) comprising the steps of:
(a) receiving a tumor or tumor fragment from a patient, the tumor or tumor fragment comprising a first population of TILs;
(b) optionally fragmenting the tumor to obtain tumor fragments;
(c) contacting the tumor fragments with a first cell culture medium;
(d) performing an initial expansion of the first population of TILs in the first cell culture medium to obtain a second population of TILs, wherein the second population of TILs is at least 5-fold greater in number than the first population of TILs, wherein the first cell culture medium comprises IL-2;
(e) performing a rapid expansion of the second population of TILs in a second cell culture medium to obtain a third population of TILs, wherein the third population of TILs is at least 50-fold greater in number than the second population of TILs after 7 days from the start of the rapid expansion; wherein the second cell culture medium comprises IL-2, OKT-3 (anti-CD3 antibody), and irradiated allogeneic peripheral blood mononuclear cells (PBMCs); and wherein the rapid expansion is performed over a period of 14 days or less; and
(f) harvesting the third population of TILs;
   wherein the cancer is selected from the group consisting of non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁺/PR⁺/HER2⁺), triple negative breast cancer (ER⁻/PR⁻/HER2⁻), double-refractory melanoma, and uveal (ocular) melanoma. In some embodiments, the method further comprises administering a therapeutically effective portion of the third population of TILs to a patient with the cancer.

Efficacy of the methods, compounds, and combinations of compounds described herein in treating, preventing and/or managing the indicated diseases or disorders can be tested using various animal models known in the art. Models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy of treatments for colorectal cancer, including the CT26 model, are described in Castle, et al., BMC Genomics, 2013, 15, 190; Endo, et al., Cancer Gene Therapy, 2002, 9, 142-148; Roth, et al., Adv. Immunol. 1994, 57, 281-351; Fearon, et al., Cancer Res. 1988, 48, 2975-2980.

Non-Myeloablative Lymphodepletion with Chemotherapy

In an embodiment, the invention provides a method of treating a cancer with a population of T cells or TILs, wherein a patient is pre-treated with non-myeloablative chemotherapy prior to an infusion of T cells or TILs. In an embodiment, the non-myeloablative chemotherapy is one or more chemotherapeutic agents. In an embodiment, the non-myeloablative chemotherapy is cyclophosphamide 60 mg/kg/d for 2 days (days 27 and 26 prior to T cells or TILs infusion) and fludarabine 25 mg/m²/d for 5 days (days 27 to 23 prior to TIL infusion). In an embodiment, after non-myeloablative chemotherapy and T cells or TILs infusion (at day 0) according to the present disclosure, the patient receives an intravenous infusion of IL-2 intravenously at 720,000 IU/kg every 8 hours to physiologic tolerance. In some embodiments, the T cells include tumor infiltrating lymphocytes (TILs). In some embodiments, the T cells include natural killer T cells. In some embodiments, the T cells include T helper cells. In some embodiments, the T cells include cytotoxic T cells. In some embodiments, the T cells include gamma delta T cells. In some embodiments, the T cells include allogeneic T cells. In some embodiments, the T cells include autologous T cells.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the T cells or TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/mL-10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL-10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide are together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: The BDX008 and IL2 Immunotherapy Tests

Adoptive cell transfer therapy can lead to durable complete regressions in patients with metastatic melanoma (Goff et al., J Clin Oncol, 2016, Jul. 10, 34(20):2389), but only in a minority of treated patients. It is of interest to be able to identify patients most likely or very unlikely to respond to such therapy to provide an enhanced durable response rate within a selected patient population.

Pretreatment serum samples collected from patients with metastatic melanoma in a prospective study of adoptive transfer of tumor-infiltrating lymphocytes following different intensities of lymphodepletion were provided. Two Biodesix immunotherapy tests were performed on the samples, respectively referred to herein as the BDX008 test and the IL2 test, and a new classifier development was carried out with the aim of creating a new test able to identify patients most likely to have durable benefit from therapy. The BDX008 test classifies samples as BDX008− or BDX008+. BDX008+ is more generally associated with longer periods of progression free survival than BDX008−. The IL2 test classifies samples as IL2 test Early (worse prognosis group) or IL2 test Late (better prognosis group). In other words IL2 test late is more generally associated with longer periods of progression free survival than IL2 test early.

Samples: the sample manifest included 90 samples. One sample (NV12_SP_2108_002) was missing and four samples were found to be hemolyzed on visual inspection (HARV_SP_0612_001, HARV_SP_0657_002, NV12_SP_0706_001, and NV12_SP_2186001). The remaining 85 samples were prepared for spectral acquisition and deep MALDI spectra acquired. The baseline clinical characteristics of the cohort of 85 patients with samples available for analysis are summarized in Table 1 including the baseline clinical characteristics of the analysis cohort of the 85 patients.

TABLE 1

| | | | Median (Range) | |
|---|---|---|---|---|
| Age | | | 47 | (20-65) |
| | | | n (%) | |
| | Gender | Male | 55 | (65) |
| | | Female | 30 | (35) |
| | Prior Adjuvant Therapy | Yes | 33 | (39) |
| | | No | 43 | (51) |
| | | N/A | 9 | (11) |
| | Treatment Line | 1 | 29 | (34) |
| | | 2 | 18 | (21) |
| | | 3 | 16 | (19) |
| | | 4 | 9 | (11) |
| | | 5 | 4 | (5) |
| | | N/A | 9 | (11) |
| | Race | White | 80 | (94) |
| | | Black | 2 | (2) |
| | | Asian | 1 | (1) |
| | | N/A | 2 | (2) |

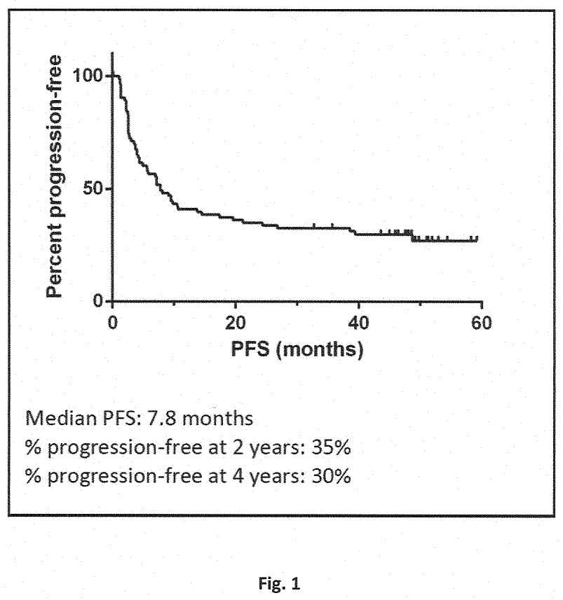

Response to therapy for the cohort is summarized in Table 2 and progression-free survival (PFS) is shown in FIG. 1 (CR: complete response; PR: partial response; NR: no response; SD: stable disease; PD: progressive disease).

TABLE 2

| Response | n (%) | |
|---|---|---|
| CR | 18 | (21) |
| Progression before 1 year | 0 | |
| Progression before 2 years | 1 | |
| Progression before 3 years | 1 | |
| Progression before 4 years | 3 | |
| PR | 28 | (33) |
| Progression before 1 year | 15 | |
| Progression before 2 years | 19 | |
| Progression before 3 years | 20 | |
| SD | 2 | (2) |
| Progression before 4 years | 0 | |
| PD | 35 | (41) |
| NR | 2 | (2) |

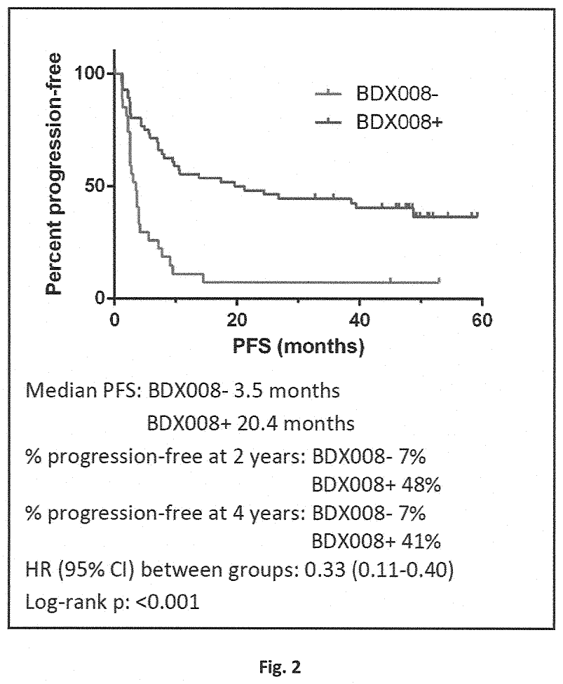

The BDX008 test was applied to the 85 samples suitable for mass spectral acquisition. Twenty-nine (34%) were classified as BDX008− and 56 (66%) as BDX008+. BDX008 classifications by sample are given in Table 3 (existing test classifications and batch allocations; batch in which mass spectra were collected, BDX008, and IL2 test classification by sample). FIG. 2 shows the Kaplan-Meier plot of PFS by BDX008 classification, and response by BDX008 classification is summarized in Table 4. Baseline characteristics by BDX008 classification are summarized in Table 5 (baseline clinical characteristics of the analysis cohort).

TABLE 3

| | | BDX008 | IL2 test |
|---|---|---|---|
| Sample ID | Batch | classification | classification |
| HARV_SP_0594_002 | Batch 1 | BDX008+ | Late |
| HARV_SP_0616_002 | Batch 1 | BDX008+ | Early |
| HARV_SP_0651_002 | Batch 2 | BDX008− | Early |
| HARV_SP_0653_002 | Batch 2 | BDX008− | Early |
| HARV_SP_0982_002 | Batch 1 | BDX008+ | Late |
| HARV_SP_1418_001 | Batch 1 | BDX008+ | Late |
| HARV_SP_1487_002 | Batch 1 | BDX008− | Early |
| HARV_SP_1490_002 | Batch 1 | BDX008+ | Late |
| HARV_SP_1506_002 | Batch 2 | BDX008+ | Late |
| HARV_SP_1592_002 | Batch 2 | BDX008+ | Early |
| HARV_SP_1709_001 | Batch 1 | BDX008− | Early |
| HARV_SP_1762_001 | Batch 1 | BDX008+ | Late |
| HARV_SP_1979_002 | Batch 1 | BDX008+ | Late |
| HARV_SP_2557_002 | Batch 2 | BDX008+ | Early |
| HARV_SP_2876_001 | Batch 1 | BDX008− | Early |
| HARV_SP_3065_001 | Batch 1 | BDX008+ | Early |
| NV12_SP_0096_002 | Batch 2 | BDX008− | Early |
| NV12_SP_0109_002 | Batch 2 | BDX008− | Early |
| NV12_SP_0142_002 | Batch 2 | BDX008+ | Late |
| NV12_SP_0157_002 | Batch 1 | BDX008− | Early |
| NV12_SP_0162_002 | Batch 1 | BDX008+ | Early |
| NV12_SP_0218_002 | Batch 2 | BDX008+ | Late |
| NV12_SP_0238_002 | Batch 2 | BDX008+ | Early |
| NV12_SP_0252_002 | Batch 1 | BDX008− | Early |
| NV12_SP_0257_002 | Batch 2 | BDX008− | Early |
| NV12_SP_0278_002 | Batch 2 | BDX008+ | Early |
| NV12_SP_0370_002 | Batch 1 | BDX008+ | Early |
| NV12_SP_0382_002 | Batch 1 | BDX008+ | Late |
| NV12_SP_0401_002 | Batch 2 | BDX008+ | Early |
| NV12_SP_0429_002 | Batch 2 | BDX008+ | Late |
| NV12_SP_0492_001 | Batch 2 | BDX008− | Early |
| NV12_SP_0495_002 | Batch 2 | BDX008+ | Early |
| NV12_SP_0572_002 | Batch 2 | BDX008− | Early |
| NV12_SP_0595_002 | Batch 1 | BDX008+ | Early |
| NV12_SP_0597_002 | Batch 1 | BDX008+ | Late |
| NV12_SP_0640_002 | Batch 2 | BDX008− | Early |
| NV12_SP_0745_001 | Batch 1 | BDX008+ | Early |
| NV12_SP_0754_002 | Batch 2 | BDX008+ | Late |
| NV12_SP_0768_002 | Batch 2 | BDX008+ | Late |
| NV12_SP_0792_002 | Batch 2 | BDX008+ | Late |
| NV12_SP_0841_002 | Batch 1 | BDX008+ | Early |
| NV12_SP_0872_001 | Batch 2 | BDX008+ | Late |
| NV12_SP_0935_002 | Batch 1 | BDX008+ | Early |
| NV12_SP_0961_002 | Batch 2 | BDX008+ | Early |
| NV12_SP_1014_002 | Batch 1 | BDX008+ | Early |
| NV12_SP_1034_002 | Batch 1 | BDX008− | Early |
| NV12_SP_1097_002 | Batch 1 | BDX008− | Early |
| NV12_SP_1104_002 | Batch 1 | BDX008− | Early |
| NV12_SP_1109_002 | Batch 1 | BDX008+ | Early |
| NV12_SP_1132_002 | Batch 2 | BDX008+ | Early |
| NV12_SP_1223_002 | Batch 1 | BDX008+ | Late |
| NV12_SP_1249_002 | Batch 1 | BDX008− | Early |
| NV12_SP_1268_002 | Batch 2 | BDX008+ | Early |
| NV12_SP_1333_002 | Batch 2 | BDX008+ | Early |
| NV12_SP_1335_002 | Batch 2 | BDX008− | Early |
| NV12_SP_1354_002 | Batch 1 | BDX008− | Early |
| NV12_SP_1419_002 | Batch 2 | BDX008− | Early |
| NV12_SP_1433_002 | Batch 2 | BDX008+ | Early |
| NV12_SP_1434_002 | Batch 1 | BDX008− | Early |
| NV12_SP_1454_002 | Batch 1 | BDX008+ | Early |
| NV12_SP_1495_002 | Batch 1 | BDX008+ | Early |
| NV12_SP_1534_002 | Batch 2 | BDX008+ | Late |
| NV12_SP_1562_001 | Batch 2 | BDX008+ | Late |
| NV12_SP_1563_002 | Batch 2 | BDX008+ | Early |
| NV12_SP_1566_001 | Batch 1 | BDX008− | Early |
| NV12_SP_1637_001 | Batch 1 | BDX008+ | Late |
| NV12_SP_1639_002 | Batch 2 | BDX008+ | Late |
| NV12_SP_1683_002 | Batch 1 | BDX008+ | Late |
| NV12_SP_1703_002 | Batch 1 | BDX008− | Early |
| NV12_SP_1726_001 | Batch 2 | BDX008− | Early |
| NV12_SP_1732_001 | Batch 1 | BDX008+ | Late |
| NV12_SP_1767_002 | Batch 1 | BDX008+ | Early |
| NV12_SP_1790_002 | Batch 2 | BDX008− | Early |
| NV12_SP_1830_001 | Batch 1 | BDX008− | Early |
| NV12_SP_1857_002 | Batch 2 | BDX008+ | Late |
| NV12_SP_1891_002 | Batch 2 | BDX008+ | Early |
| NV12_SP_1892_002 | Batch 1 | BDX008+ | Late |
| NV12_SP_1934_002 | Batch 2 | BDX008+ | Late |
| NV12_SP_1936_002 | Batch 1 | BDX008− | Early |
| NV12_SP_1996_001 | Batch 2 | BDX008− | Early |
| NV12_SP_2003_002 | Batch 1 | BDX008+ | Early |
| NV12_SP_2062_001 | Batch 2 | BDX008+ | Early |
| NV12_SP_2078_002 | Batch 2 | BDX008+ | Early |
| NV12_SP_2144_001 | Batch 2 | BDX008− | Early |
| NV12_SP_2189_001 | Batch 1 | BDX008+ | Early |

TABLE 4

| | | |
|---|---|---|
| | BDX008− | BDX008+ |
| Response | n (%) | n (%) |

| | | | | |
|---|---|---|---|---|
| CR | 3 | (10) | 15 | (27) |

| | | |
|---|---|---|
| Progression before 1 year | 0 | 0 |
| Progression before 2 years | 1 | 0 |
| Progression before 3 years | 1 | 0 |
| Progression before 4 years | 1 | 2 |

| | | | | |
|---|---|---|---|---|
| PR | 7 | (24) | 21 | (38) |

| | | |
|---|---|---|
| Progression before 1 year | 7 | 8 |
| Progression before 2 years | 7 | 12 |
| Progression before 3 years | 7 | 13 |

| | | | | |
|---|---|---|---|---|
| SD | 0 | (0) | 2 | (4) |
| PD | 17 | (59) | 18 | (32) |
| NE | 2 | (7) | 0 | (0) |

TABLE 5

| | | BDX008− | | BDX008+ | |
|---|---|---|---|---|---|
| | | Median (Range) | | | |
| Age | | 47 | (21-62) | 46 | (20-65) |
| | | n (%) | | | |
| Gender | Male | 19 | (66) | 36 | (64) |
| | Female | 10 | (34) | 20 | (36) |
| Prior Adjuvant Therapy | Yes | 12 | (41) | 21 | (38) |
| | No | 14 | (48) | 29 | (52) |
| | N/A | 3 | (10) | 6 | (11) |
| Treatment Line | 1 | 8 | (28) | 21 | (38) |
| | 2 | 7 | (24) | 11 | (20) |
| | 3 | 5 | (17) | 11 | (20) |
| | 4 | 4 | (14) | 5 | (9) |
| | 5 | 2 | (7) | 2 | (4) |
| | N/A | 3 | (10) | 6 | (11) |
| Race | White | 25 | (86) | 55 | (98) |
| | Black | 2 | (7) | 0 | (0) |
| | Asian | 0 | (0) | 1 | (2) |
| | N/A | 2 | (7) | 0 | (0) |

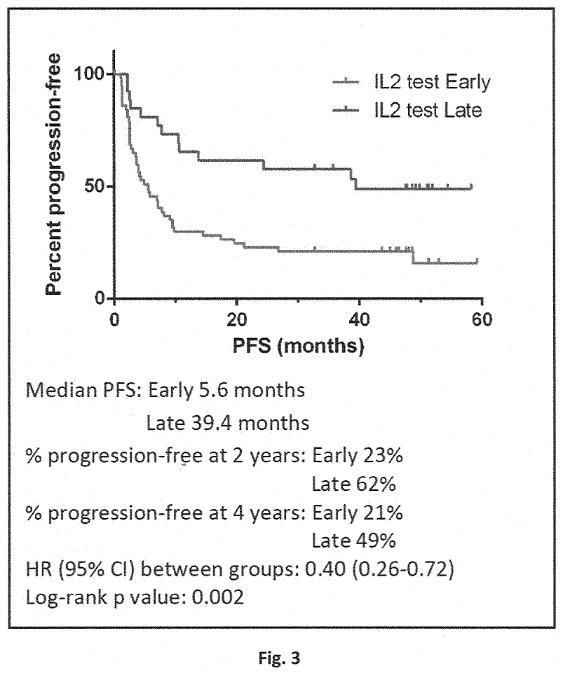

The IL2 test was applied to the 85 samples suitable for mass spectral acquisition. Fifty-nine (69%) were classified as IL2 test Early (worse prognosis group) and 26 (31%) as IL2 test Late (better prognosis group). IL2 test classifications by sample are given in Table 3. FIG. 3 shows the Kaplan-Meier plot of PFS by IL2 test classification, and response by IL2 test classification is summarized in Table 6. Baseline clinical characteristics by IL2 test classification are summarized in Table 7.

TABLE 6

| | | |
|---|---|---|
| | IL2 test Early | IL2 test Late |
| Response | n (%) | n (%) |

| CR | 7 | (12) | 11 | (42) |
|---|---|---|---|---|
| Progression before 1 year | 0 | | 0 | |
| Progression before 2 years | 1 | | 0 | |
| Progression before 3 years | 1 | | 0 | |
| Progression before 4 years | 1 | | 2 | |
| PR | 20 | (34) | 8 | (31) |
| Progression before 1 year | 12 | | 3 | |
| Progression before 2 years | 15 | | 4 | |
| Progression before 3 years | 15 | | 5 | |
| SD | 1 | (2) | 1 | (4) |
| PD | 29 | (49) | 6 | (23) |
| NE | 2 | (3) | 0 | (0) |

TABLE 7

| | | IL2 test Early | | IL2 test Late | |
|---|---|---|---|---|---|
| | | Median (Range) | | | |
| Age | | 47 | (20-65) | 47 | (30-60) |
| | | n (%) | | | |
| Gender | Male | 37 | (63) | 18 | (69) |
| | Female | 22 | (37) | 8 | (31) |
| Prior Adjuvant Therapy | Yes | 24 | (41) | 9 | (35) |
| | No | 30 | (51) | 13 | (50) |
| | N/A | 5 | (8) | 4 | (15) |
| Treatment Line | 1 | 20 | (34) | 9 | (35) |
| | 2 | 10 | (17) | 8 | (31) |
| | 3 | 13 | (22) | 3 | (12) |
| | 4 | 7 | (12) | 2 | (8) |
| | 5 | 4 | (7) | 0 | (0) |
| | N/A | 5 | (8) | 4 | (15) |
| Race | White | 55 | (93) | 25 | (96) |
| | Black | 2 | (3) | 0 | (0) |
| | Asian | 0 | (0) | 1 | (4) |
| | N/A | 2 | (3) | 0 | (0) |

### Example 2: New Classifier Development

### Sample Preparation

Samples were thawed and 3 μL aliquots of each sample and quality control serum (a pooled sample obtained from serum of five healthy patients, purchased from ProMedDx, “SerumP3”) spotted onto VeriStrat® serum cards (Therapak). The cards were allowed to dry for 1 hour at ambient temperature after which the whole serum spot was punched out with a 6 mm skin biopsy punch (Acuderm). Each punch was placed in a centrifugal filter with 0.45 μm nylon membrane (VWR). One hundred μL of HPLC grade water (JT Baker) was added to the centrifugal filter containing the punch. The punches were vortexed gently for 10 minutes, then spun down at 14,000 rcf for two minutes. The flow-through was removed and transferred back on to the punch for a second round of extraction. For the second round of extraction, the punches were vortexed gently for three minutes, then spun down at 14,000 rcf for two minutes. Twenty microliters of the filtrate from each sample was then transferred to a 0.5 mL Eppendorf tube for MALDI analysis.

All subsequent sample preparation steps were carried out in a custom designed humidity and temperature control chamber (Coy Laboratory). The temperature was set to 30° C. and the relative humidity at 10%.

An equal volume of freshly prepared matrix (25 mg of sinapinic acid per 1 mL of 50% acetonitrile: 50% water plus 0.1% TFA) was added to each 20 μL serum extract and the mix vortexed for 30 sec. The first three aliquots (3×2 μL) of sample:matrix mix were discarded into the tube cap. Eight aliquots of 2 μL sample:matrix mix were then spotted onto a stainless steel MALDI target plate (SimulTOF). The MALDI target was allowed to dry in the chamber before placement in the MALDI mass spectrometer.

This set of samples was processed for MALDI analysis in two batches. QC samples were added to the beginning (two preparations) and end (two preparations) of each batch run. The distribution of the samples run by batch is shown in Table 3.

The entire sample preparation and spectral acquisition process was repeated twice for all 85 samples suitable for generation of mass spectra, with a mass spectrometer qualification run before the first run, between the first run and the second run, and immediately following the second run. Samples were randomized separately for batch and MALDI plate spot for each run.

Mass Spectrometer Qualification: The instrument qualification procedure was conducted before and after acquiring spectra from the samples, ensuring expected performance was maintained throughout data collection on the mass spectrometry for the project. The procedure is defined below.

Sample set: the RuO40 sample set is composed of 40 human serum samples that are well characterized. A ‘gold standard’ or baseline run was acquired on the ST100 mass spectrometer using the deep MALDI sample preparation and acquisition procedure. The data were processed using an established processing method, independent of any ‘test’, and a feature table of expected values was generated for 90 mass spectral features that were selected to cover the m/z range of interest and cover the range of feature intensities.

Concordance analysis: to assess instrument performance, the concordance analysis is performed on the RuO40 sample set. In brief, the samples are prepared and spectra acquired. The data is then processed using the established processing methods, including background subtraction, normalization, alignment, and batch correction (to the gold standard) to arrive at a table of feature values. These 90 features are compared to the values that were collected in the gold standard run. Concordance plots are generated and linear regressions are performed for all 90 features. The slopes are used to compute a summary statistic (essentially a sum of residuals squared). To pass the concordance analysis the summary statistic must meet the requirements of an established metric (summary statistic >0.96). In addition, the spectra must pass all quality control measures that are included in the processing as a prerequisite to the concordance analysis.

Instrument Qualification Metrics for All Qualification Runs: as summarized in Table 8 and Table 9.

TABLE 8

| | Pass/Fail | Pass/Fail | Pass/Fail |
|---|---|---|---|
| | Before | After | After |
| | Running | Run 1 of | Run 2 of |
| Metric | Samples | Samples | Samples |
| Sufficient high quality raster | Pass | Pass | Pass |
| spectra collected for all | | | |
| reference samples | | | |
| Selection of pair of reference | Pass | Pass | Pass |
| spectra for batch correction | | | |
| Batch correction parameters | Pass | Pass | Pass |
| within specified limits | | | |

TABLE 9

| | Pass/Fail | Pass/Fail | Pass/Fail |
|---|---|---|---|
| | Before | After | After |
| | Run 1 of | Run 1 of | Run 2 of |
| Metric | Samples | Samples | Samples |
| Visual inspection of spectral | Pass | Pass | Pass |
| batch | | | |
| Visual inspection of | Pass | Pass | Pass |
| concordance plots compared | | | |
| to Gold Standard | | | |
| Summary statistic above | Pass | Pass | Pass |
| threshold (0.96) | | | |

### Spectral Acquisition

MALDI spectra were obtained using a MALDI-TOF mass spectrometer (SimulTOF 100 s/n: LinearBipolar 11.1024.01 from Virgin Instruments, Marlborough, Mass., USA). The instrument was set to operate in positive ion mode, with ions generated using a 349 nm, diode-pumped, frequency-tripled Nd:YLF laser operated at a laser repetition rate of 0.5 kHz. Immediately prior to each run of the test samples, the mass spectrometer underwent and passed machine qualification procedures to verify adequate mass spectrometer performance (see Table 8 and Table 9). External calibration was performed using the following peaks in the QC serum spectra: m/z=3317 Da, 4155 Da, 6635 Da, 9430 Da, 13888 Da, 15876 Da, and 28098 Da. After the second run of the test samples, the mass spectrometer again underwent and passed machine qualification.

Spectra from each MALDI spot were collected as 800 shot spectra that were ‘hardware averaged’ as the laser fires continuously across the spot while the stage is moving at a speed of 0.25 mm/sec. A minimum intensity threshold of 0.01 V was used to discard any ‘flat line’ spectra. All 800 shot spectra with intensity above this threshold were acquired without any further processing.

### Spectral Processing

For the new classifier development, spectral processing parameters were defined specifically.

Raster Spectral Processing—Alignment and filtering: all raster spectra of 800 shots were processed through an alignment workflow to align prominent peaks in the spectra to a set of 43 alignment points (see Table 10). A filter was applied that smooths noise and background was subtracted for peak identification. Given the identified peaks, the filtered spectra (without background subtraction) were aligned. Additional filtering parameters required that raster spectra have at least 20 peaks and used at least 5 alignment points to be included in the pool of rasters used to assemble the average spectrum.

TABLE 10
m/z

3168.00
4153.48
4183.00
4792.00
5773.00
5802.00
6432.79
6631.06
7202.00
7563.00
7614.00
7934.00
8034.00
8206.35
8684.25
8812.00
8919.00
8994.00
9133.25
9310.00
9427.00
10739.00
10938.00
11527.06
12173.00
12572.38
12864.24
13555.00
13762.87
13881.55
14039.60
14405.00
15127.49
15263.00
15869.06
17253.06
18629.76
21065.65
23024.00
28090.00
28298.00

Raster Averaging: averages were created from the pool of aligned and filtered raster spectra. A random selection of 500 raster spectra was averaged to create a final analysis spectrum for each sample of 400,000 shots.

### Average Spectra Processing

Load range: although spectra are typically collected in the m/z range of 3-75 kDa, the range for spectral processing, including feature generation, is limited to 3-30 kDa, as features above 30 kDa have poor resolution and have been found not to be reproducible at a feature value level.

Background estimation and subtraction: the Eilers method of background estimation was implemented following a preliminary analysis which identified the background estimation method superior to the standard two window method used in pre-processing for the IL2 test and BDX008. The selected parameters managed background across the m/z of interest, through all peak intensities, and reasonably well within peak clusters (where the improvement over the two window method is best observed).

TABLE 11
Background estimation Eilers parameters

| Log₁₀ (lambda) | P |
|---|---|
| 4.0 | 0.001 |

Normalization by bin method: the bin method was used to compare clinical groups of interest to ensure that normalization windows are not selected that have desirable characteristics for distinguishing the groups of interest. The windows, or bins, capture regions of similar behavior in the spectra. For example, peak clusters are contained within a single bin rather than evaluating single peaks individually. The initial normalization bin definitions can be found in Table 12. With the limited m/z range of interest, normalization bins greater than 30 kDa were excluded in the normalization bin analysis. As a second step, the normalization windows were reduced using the many replicates of reference samples that are spotted alongside test samples on every batch, which serve as quality control and for batch corrections, to remove bins that are intrinsically unstable. To do this, we evaluated the CVs of all bins for 160 reference replicates collected in 40 batches. A CV cutoff of 0.18 was applied. Bins with CVs greater than 0.18 were no longer considered for normalization as these surpassed the threshold. This reduced the normalization bins from 77 to 58 bins. The reduced set of bins can be found in Table 13.

TABLE 12
Normalization bins for serum samples

| Left | Right |
|---|---|
| 3191.81 | 3334.77 |
| 3335.74 | 3529.56 |
| 3530.68 | 3784.66 |
| 3785.03 | 4078.74 |
| 4078.78 | 4218.00 |
| 4220.21 | 4323.06 |
| 4323.55 | 4488.03 |
| 4488.68 | 4693.95 |
| 4695.90 | 4732.16 |
| 4732.81 | 4872.68 |
| 4873.33 | 5120.69 |
| 5123.93 | 5258.62 |
| 5260.63 | 5435.52 |
| 5436.47 | 5682.43 |
| 5683.32 | 6038.99 |
| 6050.42 | 6376.81 |
| 6377.92 | 6510.48 |
| 6510.85 | 6601.08 |
| 6602.94 | 6712.85 |
| 6713.22 | 6819.79 |
| 6821.17 | 7067.24 |
| 7069.18 | 7226.54 |
| 7229.72 | 7513.77 |
| 7544.49 | 7689.54 |
| 7690.19 | 7835.24 |
| 7839.13 | 7918.13 |
| 7918.78 | 8071.60 |
| 8073.54 | 8255.51 |
| 8262.34 | 8398.24 |
| 8402.18 | 8498.62 |
| 8499.51 | 8854.29 |
| 8856.95 | 9051.60 |
| 9054.25 | 9171.92 |
| 9172.81 | 9271.02 |
| 9274.56 | 9546.18 |
| 9547.95 | 9811.60 |
| 9816.95 | 10014.67 |
| 10037.22 | 10322.11 |
| 10324.76 | 10605.23 |
| 10606.12 | 10897.20 |
| 10908.61 | 11356.51 |
| 11357.83 | 12206.57 |
| 12217.91 | 12419.61 |
| 12425.27 | 12527.26 |
| 12528.39 | 13198.09 |
| 13202.62 | 13552.77 |
| 13557.04 | 13700.15 |
| 13701.44 | 14009.03 |
| 14012.92 | 14322.45 |
| 14342.46 | 14882.16 |
| 14886.47 | 14997.85 |
| 14998.49 | 15096.92 |
| 15100.81 | 15366.31 |
| 15372.78 | 15712.75 |
| 15714.69 | 15835.13 |
| 15837.08 | 16117.47 |
| 16120.06 | 16419.88 |
| 16420.16 | 16968.56 |
| 16993.85 | 17629.27 |
| 17710.35 | 18504.69 |
| 18505.83 | 19206.12 |
| 19212.92 | 20743.82 |
| 20746.09 | 21629.96 |
| 21632.22 | 22107.02 |
| 22108.95 | 22959.15 |
| 22962.61 | 23722.95 |
| 23738.50 | 24739.04 |
| 24755.19 | 27210.46 |
| 27497.49 | 29918.19 |
| 30042.68 | 32096.81 |
| 32930.23 | 35409.71 |
| 35433.92 | 38598.12 |
| 38618.86 | 41098.35 |
| 41218.18 | 48614.01 |
| 48689.99 | 55292.20 |
| 55511.71 | 63329.67 |
| 63490.08 | 72599.78 |

TABLE 13
Normalization bins for serum samples, excluding high m/z
bins and reduced by reference replicate CV threshold

| Left | Right |
|---|---|
| 3191.81 | 3334.77 |
| 3785.03 | 4078.74 |
| 4078.78 | 4218.00 |
| 4220.21 | 4323.06 |
| 4323.55 | 4488.03 |
| 4488.68 | 4693.95 |
| 4695.90 | 4732.16 |
| 4732.81 | 4872.68 |
| 4873.33 | 5120.69 |
| 5123.93 | 5258.62 |
| 5260.63 | 5435.52 |
| 5683.32 | 6038.99 |
| 6050.42 | 6376.81 |
| 6377.92 | 6510.48 |
| 6510.85 | 6601.08 |
| 6602.94 | 6712.85 |
| 6713.22 | 6819.79 |
| 6821.17 | 7067.24 |
| 7069.18 | 7226.54 |
| 7229.72 | 7513.77 |
| 7544.49 | 7689.54 |
| 7690.19 | 7835.24 |
| 7839.13 | 7918.13 |
| 7918.78 | 8071.60 |
| 8073.54 | 8255.51 |
| 8262.34 | 8398.24 |
| 8402.18 | 8498.62 |
| 8499.51 | 8854.29 |
| 8856.95 | 9051.60 |
| 9054.25 | 9171.92 |
| 9172.81 | 9271.02 |
| 9274.56 | 9546.18 |
| 9547.95 | 9811.60 |
| 9816.95 | 10014.67 |
| 10037.22 | 10322.11 |
| 10324.76 | 10605.23 |
| 10606.12 | 10897.20 |
| 10908.61 | 11356.51 |
| 11357.83 | 12206.57 |
| 12217.91 | 12419.61 |
| 12425.27 | 12527.26 |
| 12528.39 | 13198.09 |
| 13202.62 | 13552.77 |
| 13701.44 | 14009.03 |
| 14012.92 | 14322.45 |
| 14342.46 | 14882.16 |
| 14886.47 | 14997.85 |
| 14998.49 | 15096.92 |
| 15100.81 | 15366.31 |
| 15372.78 | 15712.75 |
| 15837.08 | 16117.47 |
| 16120.06 | 16419.88 |
| 16420.16 | 16968.56 |
| 17710.35 | 18504.69 |
| 18505.83 | 19206.12 |
| 20746.09 | 21629.96 |
| 21632.22 | 22107.02 |
| 27497.49 | 29918.19 |

To further prune the normalization windows, dependence on response category was used to evaluate CVs and univariate p values. Using the samples in the development set (n=85), the samples from patients achieving a complete response (CR, n=18) were compared to all other patient spectra (partial response, no response, stable disease, or progressive disease) to compute univariate p values for each of the bins. This approach was used to remove normalization windows that may be important for distinguishing the clinical groups CR vs other. A p value cutoff of 0.20 was applied (bins with p values below 0.20 were rejected) and a CV cutoff of 0.25 (bins above 0.25 were rejected). The list of normalization windows was reduced to 11 bins that can be found in Table 14.

TABLE 14
Normalization by bin windows

| Left Limit (m/z) | Right Limit (m/z) |
|---|---|
| 3785.03 | 4078.74 |
| 4732.81 | 4872.68 |
| 5260.63 | 5435.52 |
| 6510.85 | 6601.08 |
| 10606.12 | 10897.20 |
| 10908.61 | 11356.51 |
| 13202.62 | 13552.77 |
| 14998.49 | 15096.92 |
| 16420.16 | 16968.56 |
| 17710.35 | 18504.69 |
| 18505.83 | 19206.12 |

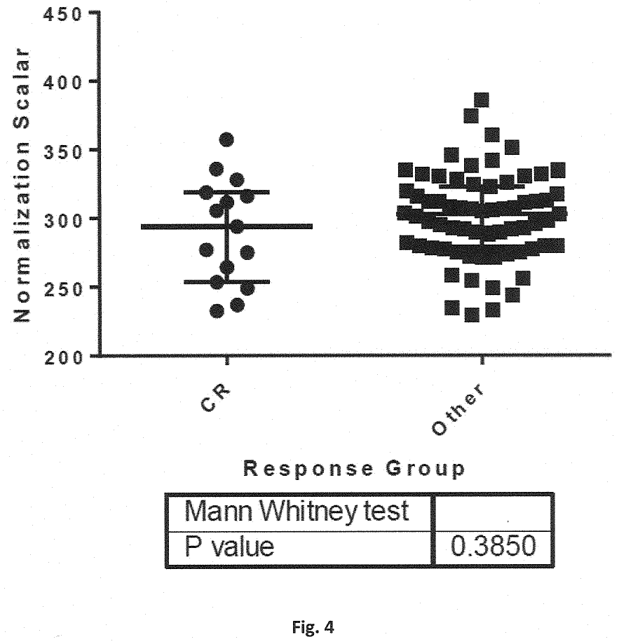

The resulting normalization scalars were compared between the response groups to ensure the combination of windows was not significantly associated with the clinical groups. The plot in FIG. 4 demonstrates that the distribution of normalization scalars was not associated with the clinical groups of interest.

Average spectra alignment: the peak alignment of the average spectra is typically very good; however, a fine-tune alignment step was performed to address minor differences in peak positions in the spectra. A set of 26 alignment points was identified and applied to the analysis spectra (Table 15) using a calibration tolerance of 800 ppm. The range of interest for calibration was limited to 3-32 kDa.

TABLE 15
Calibration points used to align the spectral averages
m/z

3315.17
4153.33
4456.88
4709.91
5066.47
6432.85
6631.27
7934.36
8916.29
9423.10
9714.25
12868.19
13766.39
14044.69
14093.30
15131.43
15871.93
16077.64
17255.58
17383.45
18630.93
21069.05
21168.45
28084.44
28292.86
67150.37

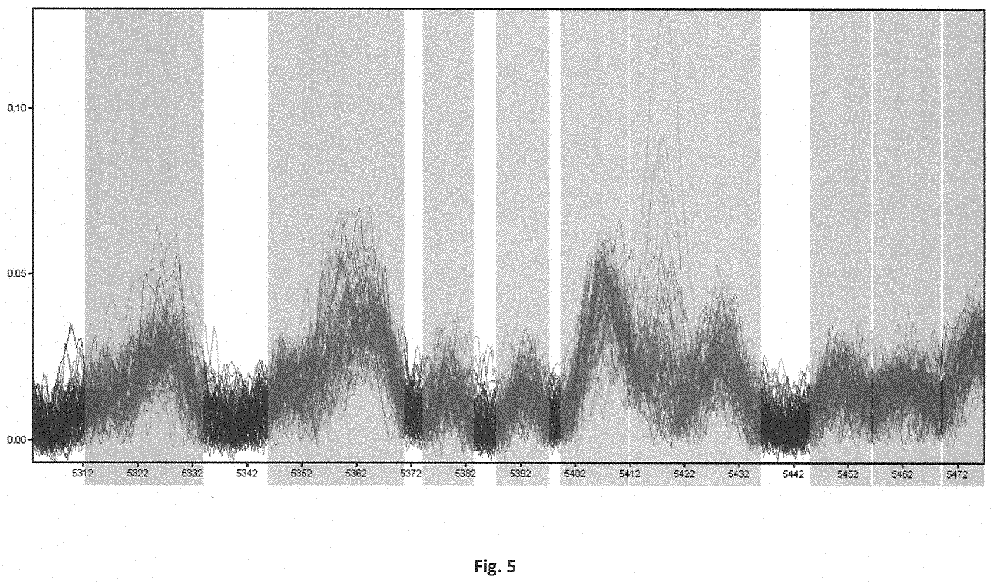

Feature Definitions were selected by comparing spectra from each clinical group (defined by CR or other). Several features were identified that may have heightened susceptibility to peptide modifications that take place during the sample preparation procedure. These manifest themselves in specific m/z regions of the spectra where the peaks change in intensity and shape and may depend on the position on the plate where the sample was spotted. These m/z regions were excluded from feature selection. A final set of 418 feature definitions were applied to the spectra, and these are listed in Table 16. An example of features defined using the described method is displayed in FIG. 5 with reference spectra shown in blue and spectra from batch 1 of test samples in red. Each turquoise highlighted region represents a separate feature definition. The feature value for a specific spectrum is the area under the spectrum within the m/z span of the feature definition.

TABLE 16
Feature Definitions (m/z)

| Left Limit | Center | Right Limit |
|---|---|---|
| 3069.03 | 3080.45 | 3091.87 |
| 3094.37 | 3105.25 | 3116.14 |
| 3118.81 | 3124.60 | 3130.38 |
| 3130.59 | 3137.59 | 3144.60 |
| 3146.02 | 3153.23 | 3160.44 |
| 3161.66 | 3173.54 | 3185.42 |
| 3189.89 | 3195.78 | 3201.67 |
| 3204.58 | 3214.03 | 3223.47 |
| 3229.15 | 3238.31 | 3247.47 |
| 3254.65 | 3261.38 | 3268.10 |
| 3273.55 | 3285.08 | 3296.61 |
| 3303.45 | 3312.44 | 3321.42 |
| 3339.04 | 3345.94 | 3352.83 |
| 3353.18 | 3364.25 | 3375.32 |
| 3384.94 | 3393.34 | 3401.75 |
| 3408.24 | 3414.44 | 3420.64 |
| 3420.87 | 3425.63 | 3430.38 |
| 3434.43 | 3442.26 | 3450.08 |
| 3454.37 | 3460.52 | 3466.66 |
| 3467.47 | 3477.67 | 3487.87 |
| 3495.64 | 3502.36 | 3509.08 |
| 3509.20 | 3516.33 | 3523.46 |
| 3524.38 | 3531.69 | 3538.99 |
| 3539.80 | 3551.45 | 3563.10 |
| 3577.94 | 3588.37 | 3598.80 |
| 3603.78 | 3610.56 | 3617.35 |
| 3621.75 | 3628.70 | 3635.66 |
| 3647.71 | 3654.32 | 3660.93 |
| 3667.54 | 3678.72 | 3689.91 |
| 3694.08 | 3701.90 | 3709.73 |
| 3713.78 | 3722.01 | 3730.24 |
| 3732.68 | 3738.18 | 3743.69 |
| 3746.01 | 3753.37 | 3760.73 |
| 3764.44 | 3773.07 | 3781.71 |
| 3786.23 | 3792.55 | 3798.86 |
| 3803.27 | 3815.32 | 3827.38 |
| 3832.25 | 3838.51 | 3844.77 |
| 3884.57 | 3889.69 | 3894.80 |
| 3898.51 | 3904.77 | 3911.03 |
| 3913.99 | 3919.35 | 3924.70 |
| 3926.45 | 3931.70 | 3936.95 |
| 3942.54 | 3949.78 | 3957.02 |
| 3970.01 | 3975.03 | 3980.04 |
| 3980.72 | 3986.47 | 3992.23 |
| 4001.18 | 4010.81 | 4020.44 |
| 4025.02 | 4029.93 | 4034.85 |
| 4039.09 | 4050.43 | 4061.78 |
| 4066.83 | 4073.59 | 4080.36 |
| 4083.93 | 4096.02 | 4108.10 |
| 4112.01 | 4119.11 | 4126.21 |
| 4126.48 | 4132.14 | 4137.79 |
| 4201.08 | 4206.10 | 4211.11 |
| 4220.94 | 4226.06 | 4231.18 |
| 4232.52 | 4238.05 | 4243.57 |
| 4244.17 | 4250.30 | 4256.43 |
| 4260.33 | 4265.14 | 4269.96 |
| 4274.20 | 4286.49 | 4298.78 |
| 4331.23 | 4339.88 | 4348.53 |
| 4353.58 | 4360.18 | 4366.78 |
| 4372.16 | 4378.90 | 4385.63 |
| 4385.97 | 4390.07 | 4394.18 |
| 4396.07 | 4406.47 | 4416.87 |
| 4426.50 | 4432.02 | 4437.54 |
| 4448.18 | 4459.52 | 4470.87 |
| 4485.14 | 4506.28 | 4527.42 |
| 4528.70 | 4533.79 | 4538.87 |
| 4539.95 | 4545.20 | 4550.45 |
| 4553.75 | 4564.69 | 4575.63 |
| 4579.13 | 4585.09 | 4591.05 |
| 4591.18 | 4598.29 | 4605.39 |
| 4616.63 | 4624.17 | 4631.72 |
| 4631.78 | 4634.81 | 4637.84 |
| 4638.78 | 4644.44 | 4650.10 |
| 4651.91 | 4671.24 | 4690.56 |
| 4699.58 | 4710.42 | 4721.26 |
| 4734.59 | 4740.32 | 4746.04 |
| 4748.33 | 4755.16 | 4762.00 |
| 4767.11 | 4773.91 | 4780.71 |
| 4781.12 | 4789.87 | 4798.62 |
| 4806.64 | 4817.95 | 4829.26 |
| 4846.43 | 4855.95 | 4865.48 |
| 4879.28 | 4888.51 | 4897.73 |
| 4905.07 | 4916.28 | 4927.49 |
| 4929.24 | 4936.98 | 4944.73 |
| 4952.60 | 4962.13 | 4971.66 |
| 5012.53 | 5019.06 | 5025.59 |
| 5026.80 | 5031.68 | 5036.56 |
| 5036.63 | 5042.72 | 5048.82 |
| 5055.08 | 5067.33 | 5079.59 |
| 5084.16 | 5100.66 | 5117.15 |
| 5120.69 | 5128.78 | 5136.86 |
| 5137.14 | 5148.68 | 5160.22 |
| 5164.92 | 5169.48 | 5174.04 |
| 5174.32 | 5190.22 | 5206.11 |
| 5216.20 | 5225.53 | 5234.86 |
| 5239.84 | 5249.10 | 5258.36 |
| 5273.84 | 5287.59 | 5301.35 |
| 5312.41 | 5323.19 | 5333.97 |
| 5345.72 | 5358.23 | 5370.74 |
| 5374.05 | 5378.68 | 5383.31 |
| 5387.46 | 5392.30 | 5397.14 |
| 5399.21 | 5405.36 | 5411.51 |
| 5411.79 | 5417.52 | 5423.26 |
| 5423.40 | 5429.55 | 5435.70 |
| 5444.68 | 5450.35 | 5456.02 |
| 5456.43 | 5462.58 | 5468.73 |
| 5469.01 | 5474.75 | 5480.48 |
| 5485.87 | 5491.68 | 5497.49 |
| 5498.31 | 5504.19 | 5510.06 |
| 5513.52 | 5519.88 | 5526.24 |
| 5541.16 | 5552.84 | 5564.52 |
| 5567.56 | 5573.58 | 5579.59 |
| 5581.83 | 5587.96 | 5594.09 |
| 5595.72 | 5601.47 | 5607.21 |
| 5609.83 | 5615.54 | 5621.26 |
| 5624.97 | 5635.12 | 5645.28 |
| 5669.95 | 5677.43 | 5684.92 |
| 5685.75 | 5691.45 | 5697.15 |
| 5698.10 | 5706.35 | 5714.61 |
| 5715.08 | 5720.78 | 5726.48 |
| 5726.60 | 5735.80 | 5745.01 |
| 5751.42 | 5763.24 | 5775.06 |
| 5785.98 | 5793.52 | 5801.06 |
| 5801.78 | 5814.90 | 5828.02 |
| 5829.09 | 5841.03 | 5852.96 |
| 5860.33 | 5868.94 | 5877.55 |
| 5879.92 | 5890.79 | 5901.66 |
| 5901.89 | 5907.12 | 5912.34 |
| 5916.03 | 5921.61 | 5927.19 |
| 5931.82 | 5945.30 | 5958.78 |
| 5958.90 | 5966.02 | 5973.15 |
| 5978.49 | 5987.46 | 5996.43 |
| 6000.70 | 6006.76 | 6012.82 |
| 6021.37 | 6029.80 | 6038.23 |
| 6054.03 | 6061.51 | 6068.99 |
| 6070.65 | 6076.29 | 6081.94 |
| 6082.17 | 6089.36 | 6096.54 |
| 6100.70 | 6108.30 | 6115.90 |
| 6121.25 | 6127.54 | 6133.83 |
| 6136.45 | 6149.21 | 6161.98 |
| 6164.47 | 6171.84 | 6179.20 |
| 6181.58 | 6192.03 | 6202.48 |
| 6203.55 | 6211.09 | 6218.63 |
| 6218.75 | 6224.98 | 6231.22 |
| 6234.66 | 6240.54 | 6246.42 |
| 6248.80 | 6254.56 | 6260.32 |
| 6275.04 | 6284.42 | 6293.81 |
| 6294.40 | 6302.12 | 6309.84 |
| 6323.26 | 6331.93 | 6340.60 |
| 6377.30 | 6390.89 | 6404.49 |
| 6417.32 | 6431.69 | 6446.06 |
| 6449.86 | 6455.38 | 6460.90 |
| 6518.15 | 6529.73 | 6541.31 |
| 6568.86 | 6585.96 | 6603.06 |
| 6609.00 | 6612.56 | 6616.13 |
| 6620.04 | 6632.99 | 6645.93 |
| 6649.50 | 6656.44 | 6663.39 |
| 6718.02 | 6729.36 | 6740.71 |
| 6751.99 | 6758.88 | 6765.77 |
| 6767.43 | 6774.79 | 6782.15 |
| 6785.95 | 6792.19 | 6798.42 |
| 6798.66 | 6807.33 | 6816.00 |
| 6828.35 | 6836.96 | 6845.57 |
| 6851.39 | 6859.29 | 6867.19 |
| 6870.28 | 6879.60 | 6888.92 |
| 6889.51 | 6898.30 | 6907.09 |
| 6913.50 | 6920.04 | 6926.57 |
| 6929.06 | 6939.45 | 6949.85 |
| 6950.44 | 6955.96 | 6961.48 |
| 6961.72 | 6969.08 | 6976.45 |
| 6977.04 | 6986.48 | 6995.93 |
| 6997.59 | 7005.49 | 7013.38 |
| 7013.86 | 7020.09 | 7026.33 |
| 7027.28 | 7041.53 | 7055.78 |
| 7056.97 | 7062.55 | 7068.13 |
| 7068.61 | 7074.55 | 7080.48 |
| 7080.72 | 7096.75 | 7112.79 |
| 7120.98 | 7142.24 | 7163.50 |
| 7179.77 | 7188.32 | 7196.87 |
| 7238.08 | 7243.96 | 7249.84 |
| 7253.04 | 7258.86 | 7264.68 |
| 7265.99 | 7271.81 | 7277.63 |
| 7282.85 | 7287.13 | 7291.40 |
| 7294.02 | 7299.42 | 7304.82 |
| 7309.81 | 7318.60 | 7327.39 |
| 7327.51 | 7333.56 | 7339.62 |
| 7350.90 | 7358.56 | 7366.22 |
| 7377.86 | 7389.32 | 7400.78 |
| 7406.96 | 7418.60 | 7430.24 |
| 7432.97 | 7440.75 | 7448.53 |
| 7461.59 | 7473.47 | 7485.34 |
| 7499.00 | 7506.84 | 7514.68 |
| 7526.91 | 7534.98 | 7543.06 |
| 7602.90 | 7616.21 | 7629.53 |
| 7730.47 | 7738.72 | 7746.97 |
| 7759.80 | 7767.16 | 7774.53 |
| 7776.90 | 7784.03 | 7791.15 |
| 7807.66 | 7820.55 | 7833.43 |
| 7848.16 | 7871.08 | 7894.00 |
| 7906.10 | 7912.37 | 7918.63 |
| 8126.20 | 8152.96 | 8179.71 |
| 8196.59 | 8206.30 | 8216.02 |
| 8238.46 | 8257.92 | 8277.39 |
| 8307.95 | 8315.08 | 8322.20 |
| 8324.12 | 8329.52 | 8334.92 |
| 8355.64 | 8366.35 | 8377.05 |
| 8381.08 | 8391.28 | 8401.47 |
| 8405.37 | 8412.82 | 8420.27 |
| 8423.02 | 8430.78 | 8438.55 |
| 8458.05 | 8464.06 | 8470.07 |
| 8472.18 | 8478.28 | 8484.39 |
| 8484.58 | 8490.33 | 8496.09 |
| 8500.30 | 8509.48 | 8518.65 |
| 8519.48 | 8530.48 | 8541.47 |
| 8555.60 | 8564.36 | 8573.12 |
| 8573.76 | 8591.18 | 8608.60 |
| 8650.09 | 8658.24 | 8666.39 |
| 8722.26 | 8736.80 | 8751.35 |
| 8757.16 | 8768.06 | 8778.96 |
| 8783.50 | 8787.59 | 8791.68 |
| 8792.00 | 8795.23 | 8798.46 |
| 8800.31 | 8819.84 | 8839.37 |
| 8850.37 | 8865.71 | 8881.05 |
| 8882.65 | 8891.63 | 8900.62 |
| 8905.54 | 8925.55 | 8945.56 |
| 8989.86 | 8997.37 | 9004.88 |
| 9012.62 | 9019.97 | 9027.32 |
| 9030.58 | 9037.17 | 9043.75 |
| 9057.37 | 9063.57 | 9069.77 |
| 9070.79 | 9078.53 | 9086.26 |
| 9090.10 | 9096.71 | 9103.33 |
| 9115.92 | 9133.31 | 9150.70 |
| 9236.49 | 9243.27 | 9250.04 |
| 9256.18 | 9262.76 | 9269.35 |
| 9274.46 | 9287.38 | 9300.29 |
| 9311.16 | 9319.02 | 9326.88 |
| 9343.95 | 9358.59 | 9373.23 |
| 9387.36 | 9395.22 | 9403.08 |
| 9413.18 | 9426.71 | 9440.23 |
| 9460.75 | 9468.32 | 9475.90 |
| 9476.28 | 9483.92 | 9491.56 |
| 9491.88 | 9505.24 | 9518.60 |
| 9518.66 | 9533.11 | 9547.56 |
| 9558.17 | 9583.71 | 9609.25 |
| 9619.86 | 9640.89 | 9661.93 |
| 9666.68 | 9674.09 | 9681.51 |
| 9701.69 | 9719.01 | 9736.34 |
| 9755.33 | 9788.42 | 9821.50 |
| 9834.77 | 9868.72 | 9902.66 |
| 9908.90 | 9919.05 | 9929.19 |
| 9929.66 | 9940.12 | 9950.58 |
| 10070.28 | 10079.80 | 10089.32 |
| 10091.51 | 10100.87 | 10110.24 |
| 10126.16 | 10137.63 | 10149.10 |
| 10151.28 | 10160.49 | 10169.70 |
| 10174.85 | 10185.15 | 10195.45 |
| 10201.85 | 10211.61 | 10221.36 |
| 10226.51 | 10235.95 | 10245.40 |
| 10246.96 | 10261.31 | 10275.67 |
| 10276.61 | 10284.80 | 10293.00 |
| 10294.71 | 10303.53 | 10312.35 |
| 10333.73 | 10346.30 | 10358.86 |
| 10411.92 | 10420.51 | 10429.09 |
| 10440.17 | 10448.84 | 10457.50 |
| 10463.58 | 10469.28 | 10474.98 |
| 10475.13 | 10482.39 | 10489.65 |
| 10490.59 | 10496.20 | 10501.82 |
| 10504.16 | 10510.33 | 10516.49 |
| 10522.11 | 10535.53 | 10548.96 |
| 10572.68 | 10586.26 | 10599.84 |
| 10617.32 | 10637.14 | 10656.96 |
| 10764.80 | 10774.32 | 10783.84 |
| 10795.24 | 10802.65 | 10810.06 |
| 10825.98 | 10837.46 | 10848.93 |
| 10849.24 | 10857.12 | 10865.00 |
| 10909.33 | 10921.50 | 10933.67 |
| 10953.50 | 10965.28 | 10977.06 |
| 10992.51 | 11002.58 | 11012.65 |
| 11034.50 | 11045.97 | 11057.44 |
| 11057.60 | 11066.41 | 11075.23 |
| 11094.12 | 11105.20 | 11116.28 |
| 11142.34 | 11150.46 | 11158.57 |
| 11288.89 | 11305.05 | 11321.20 |
| 11382.23 | 11392.53 | 11402.83 |
| 11436.07 | 11444.81 | 11453.55 |
| 11470.41 | 11480.16 | 11489.92 |
| 11513.48 | 11530.18 | 11546.88 |
| 11567.95 | 11577.24 | 11586.52 |
| 11615.55 | 11630.07 | 11644.58 |
| 11671.43 | 11687.19 | 11702.95 |
| 11726.05 | 11744.55 | 11763.04 |
| 11775.06 | 11785.52 | 11795.97 |
| 11826.25 | 11842.79 | 11859.34 |
| 11877.13 | 11899.29 | 11921.46 |
| 11934.10 | 11942.53 | 11950.95 |
| 11976.86 | 11998.32 | 12019.78 |
| 12084.55 | 12116.16 | 12147.76 |
| 12177.57 | 12210.42 | 12243.28 |
| 12270.28 | 12289.79 | 12309.29 |
| 12310.08 | 12319.44 | 12328.80 |
| 12405.75 | 12414.41 | 12423.07 |
| 12441.02 | 12454.29 | 12467.55 |
| 12553.70 | 12571.18 | 12588.66 |
| 12603.65 | 12614.26 | 12624.87 |
| 12651.72 | 12668.89 | 12686.05 |
| 12686.99 | 12695.81 | 12704.63 |
| 12722.26 | 12736.39 | 12750.51 |
| 12767.99 | 12781.34 | 12794.68 |
| 12794.84 | 12804.59 | 12814.34 |
| 12840.56 | 12861.63 | 12882.70 |
| 12942.17 | 12960.58 | 12979.00 |
| 13057.35 | 13079.90 | 13102.45 |
| 13123.52 | 13133.59 | 13143.66 |
| 13151.15 | 13158.56 | 13165.98 |
| 13166.60 | 13174.79 | 13182.99 |
| 13184.70 | 13195.63 | 13206.55 |
| 13266.80 | 13275.62 | 13284.43 |
| 13290.36 | 13297.54 | 13304.72 |
| 13308.78 | 13319.39 | 13330.01 |
| 13352.48 | 13364.03 | 13375.58 |
| 13402.42 | 13412.80 | 13423.18 |
| 13502.31 | 13509.80 | 13517.29 |
| 13517.61 | 13527.28 | 13536.96 |
| 13556.94 | 13570.20 | 13583.47 |
| 13601.57 | 13612.03 | 13622.49 |
| 13708.86 | 13722.38 | 13735.89 |
| 13756.04 | 13766.36 | 13776.68 |
| 13787.14 | 13797.22 | 13807.29 |
| 13837.03 | 13845.41 | 13853.79 |
| 13873.55 | 13886.25 | 13898.94 |
| 13906.78 | 13915.31 | 13923.83 |
| 13935.07 | 13942.58 | 13950.09 |
| 13964.43 | 13981.33 | 13998.24 |
| 13999.49 | 14004.19 | 14008.89 |
| 14030.01 | 14043.82 | 14057.62 |
| 14066.05 | 14071.09 | 14076.12 |
| 14081.55 | 14098.21 | 14114.88 |
| 14129.31 | 14147.23 | 14165.15 |
| 14186.27 | 14200.56 | 14214.85 |
| 14234.42 | 14254.62 | 14274.82 |
| 14286.35 | 14304.13 | 14321.90 |
| 14330.04 | 14358.42 | 14386.81 |
| 14407.64 | 14433.46 | 14459.27 |
| 14465.18 | 14485.04 | 14504.90 |
| 14516.82 | 14538.81 | 14560.80 |
| 14571.85 | 14590.83 | 14609.82 |
| 14629.29 | 14642.37 | 14655.45 |
| 14658.36 | 14670.37 | 14682.38 |
| 14682.58 | 14695.99 | 14709.41 |
| 14764.53 | 14783.42 | 14802.32 |
| 14822.38 | 14838.56 | 14854.73 |
| 14862.86 | 14880.16 | 14897.45 |
| 14959.56 | 14971.19 | 14982.83 |
| 16270.81 | 16299.57 | 16328.33 |
| 16618.02 | 16646.36 | 16674.69 |
| 16908.34 | 16930.20 | 16952.05 |
| 17008.82 | 17025.24 | 17041.67 |
| 18249.85 | 18269.48 | 18289.11 |
| 18611.95 | 18628.27 | 18644.59 |
| 18713.66 | 18726.78 | 18739.91 |
| 18740.86 | 18753.16 | 18765.45 |
| 18818.43 | 18842.79 | 18867.16 |
| 19060.63 | 19089.60 | 19118.57 |
| 19353.20 | 19372.24 | 19391.28 |
| 20766.66 | 20815.24 | 20863.83 |
| 20910.07 | 20950.46 | 20990.85 |
| 21026.56 | 21061.39 | 21096.22 |
| 21130.17 | 21164.71 | 21199.24 |
| 21229.10 | 21265.39 | 21301.68 |
| 21329.19 | 21368.12 | 21407.05 |
| 21436.32 | 21478.76 | 21521.19 |
| 21532.32 | 21580.02 | 21627.73 |
| 21666.95 | 21696.51 | 21726.07 |
| 21733.24 | 21752.72 | 21772.20 |
| 21780.07 | 21802.92 | 21825.77 |
| 21832.89 | 21854.43 | 21875.97 |
| 21882.72 | 21905.57 | 21928.42 |
| 22050.93 | 22085.02 | 22119.11 |
| 22135.97 | 22148.71 | 22161.44 |
| 22168.94 | 22183.36 | 22197.78 |
| 22201.53 | 22217.08 | 22232.62 |
| 22237.49 | 22252.10 | 22266.72 |
| 22270.46 | 22285.63 | 22300.81 |
| 22306.80 | 22322.35 | 22337.90 |
| 22339.02 | 22356.63 | 22374.24 |
| 22374.61 | 22388.47 | 22402.33 |
| 22571.67 | 22603.70 | 22635.73 |
| 22938.06 | 22961.47 | 22984.89 |
| 23004.00 | 23030.59 | 23057.19 |
| 23106.64 | 23130.43 | 23154.22 |
| 23155.35 | 23176.89 | 23198.43 |
| 23211.92 | 23242.64 | 23273.36 |
| 23319.81 | 23351.84 | 23383.87 |
| 23431.83 | 23462.73 | 23493.64 |
| 23532.98 | 23562.01 | 23591.05 |
| 23637.13 | 23665.97 | 23694.82 |
| 25154.39 | 25180.81 | 25207.22 |
| 25231.94 | 25263.22 | 25294.51 |
| 25358.94 | 25388.16 | 25417.39 |
| 25434.99 | 25470.21 | 25505.43 |
| 25522.66 | 25562.18 | 25601.71 |
| 25631.68 | 25680.57 | 25729.46 |
| 25754.56 | 25792.77 | 25830.98 |
| 25844.47 | 25873.69 | 25902.91 |
| 27916.94 | 27964.52 | 28012.10 |
| 28039.07 | 28084.97 | 28130.86 |
| 28143.97 | 28184.81 | 28225.64 |
| 28242.87 | 28292.33 | 28341.78 |
| 28354.89 | 28396.66 | 28438.43 |
| 28455.67 | 28503.06 | 28550.45 |
| 28568.81 | 28610.39 | 28651.97 |
| 28669.96 | 28718.47 | 28766.99 |
| 28823.56 | 28869.45 | 28915.34 |
| 28934.45 | 28975.10 | 29015.74 |
| 29036.72 | 29076.81 | 29116.90 |
| 29140.50 | 29185.08 | 29229.66 |
| 29238.28 | 29282.30 | 29326.32 |
| 29346.17 | 29391.31 | 29436.46 |

### Batch Correction of Analysis Spectra

Feature Reduction: a subset of 52 of the 418 features was used to select the individual reference spectra to be used for the baseline reference in batch correction and for computing the correction function used in batch correction. All 418 features were used in reference selection for all further batches. The criteria for selecting the subset were that there could only be 3 features per each m/z interval of approximately 1 kDa and that these should be representative of the intensity range within the kDa interval (i.e., represent high, medium, and low intensities). To ensure that stable features were used for batch correction, CVs over the features were computed using 160 replicate reference spectra. For each approximately 1 kDa interval, the features were ranked by CV and intensity. A visual inspection of each feature in combination with the ranked CV and the intensity demands were used to select the subset of 52 features.

Reference Spectrum Analysis: two preparations of the reference sample, SerumP3, were plated at the beginning (1,2) and end (3,4) of each batch of test samples. The purpose of these samples is to ensure that variations by batch due to slight changes in instrument performance (for example, aging of the detector) can be corrected for. The section below describes the batch correction procedure. To perform batch correction, one spectrum must serve as the reference for the batch and this is an average of the spectra from one of the preparations from the beginning and one from the end of the batch. A procedure for selecting the pair is first described.

The reference samples were preprocessed as described above. Fifty-two features were used to evaluate the possible combinations (1-3, 1-4, 2-3, 2-4). Each possible combination of replicates was compared using the function:

A=min(abs(1−ftrval1/ftrval2),abs(1−ftrval2/ftrval1))

where ftrval1 (ftrval2) is the value of a feature for the first (second) replicate of the replicate pair. This quantity A gives a measure of how similar the replicates of the pair are. For each feature, A is reported. If the value is >0.5, then the feature is determined to be discordant, or ‘Bad’. A tally of the bad features is reported for each possible combination. If the value of A is <0.1, then the feature is determined to be concordant and reported as ‘Good’. A tally of the Good features is reported for each possible combination. Using the tallies of Bad and Good features from each possible combination, we computed the ratio of Bad/Good. The combination with the lowest ratio was reported as the most similar combo and unlikely to contain any systematic or localized outlier behavior in either of the reference spectra. Finally, if no ratio can be found that is less than 0.2, then the batch is a failure. Table 17 reports the combinations that were found most similar for each batch.

TABLE 17
SerumP3 preparations found to be most similar by batch

| Batch | Combination |
|---|---|
| Run 1 Batch 1 | 1_4 |
| Run 1 Batch 2 | 1_3 |
| Run 2 Batch 1 | 1_4 |
| Run 2 Batch 2 | 2_3 |

Batch Correction: Run 1 Batch 1 was used as the baseline batch to correct all other batches. The reference spectrum was used to find the correction coefficients for each of the batches by the following procedure. Within each batch j ( ), the ratio

$${\hat{r}}_{i}^{j} = \frac{{A}_{i}^{j}}{{A}_{i}^{1}}$$

and the average amplitude Āᵢ^{j}=1/2(Aᵢ^{j}+Aᵢ¹) are defined for each i^{th} feature centered at (m/z)ᵢ, where Aᵢ^{j} is the average reference spectra amplitude of feature i in the batch being corrected and is the reference spectra amplitude of feature i in batch 1 (the reference standard). It is assumed that the ratio of amplitudes between two batches follows the dependence

r(Ā,(m/z))=(a₀+a₁ ln(Ā))+(b₀+b₁ ln(Ā))(m/z)+c₀(m/z)².

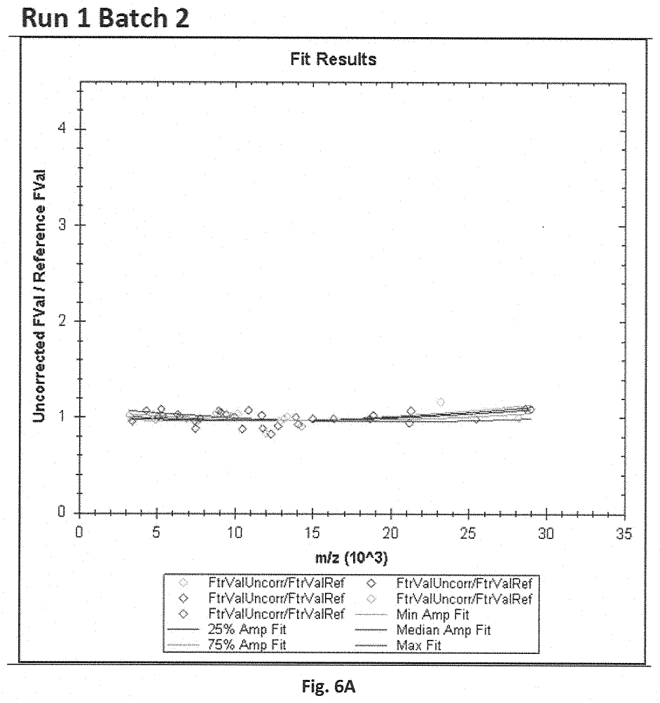

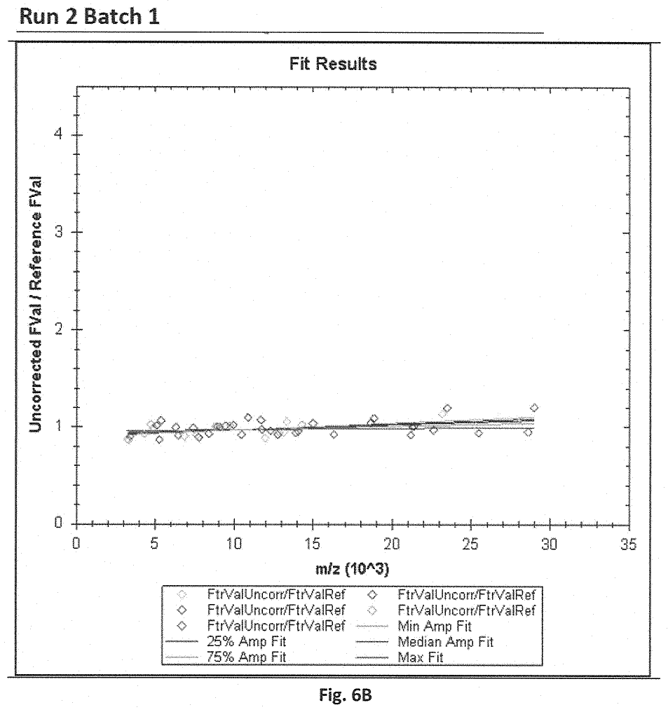

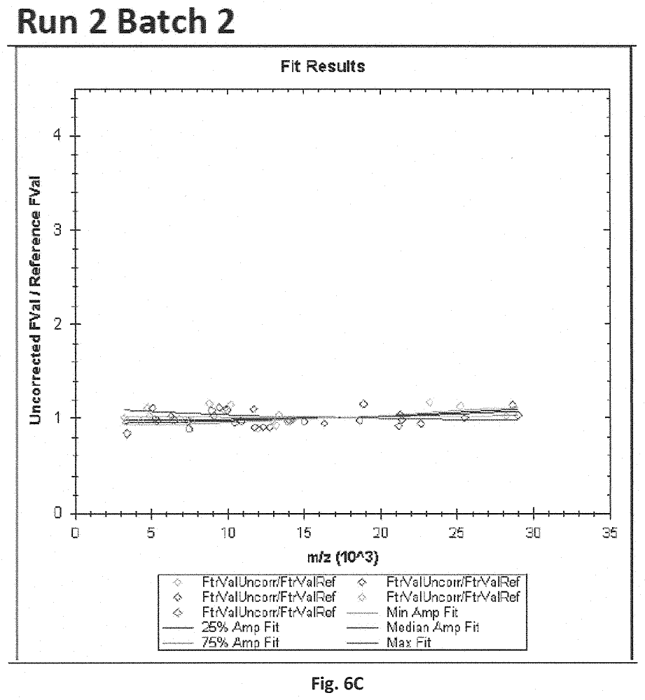

On a batch to batch basis, a continuous fit is constructed by minimizing the sum of the square residuals, Δ^{j}=Σᵢ(\hat{r}ᵢ^{j}−r^{j}(a₀,a₁,b₀,b₁,c₀))², and using the experimental data of the reference sample. The SerumP3 reference samples are used to calculate the correction function. Steps were taken to not include outlier points in order to avoid bias in the parameter estimates. The values of the coefficients a₀, a₁, b₀, b₁ and c₀, obtained for the different batches are listed in Table 18. The projection in the \hat{r}ij versus (m/z)ᵢ plane of the points used to construct the fit for each batch of reference spectra, together with the surface defined by the fit itself, is shown in FIGS. 6A, 6B, and 6C.

TABLE 18
Batch Correction coefficients pre-correction

| Model | A0 | A1 | B0 | B1 | C | ResSD |
|---|---|---|---|---|---|---|
| Run 1 Batch 2 | 1.019E+00 | 2.424E−02 | −9.192E−06 | −1.748E−06 | 4.182E−10 | 5.998E−02 |
| Run 2 Batch 1 | 9.104E−01 | 1.109E−02 | 6.083E−06 | −1.066E−06 | −8.815E−12 | 6.806E−02 |
| Run 2 Batch 2 | 9.593E−01 | 3.684E−02 | 6.837E−07 | −2.158E−06 | 1.324E−10 | 7.453E−02 |

Once the final fit, r^{j}(Ā,(m/z)), is determined for each batch, the next step is to correct, for all the samples, all 418 features (with amplitude A at (m/z)) according to

$${A}_{corr} = \frac{A}{{r}^{j} ( \overline{A} , ( m / z ) )} .$$

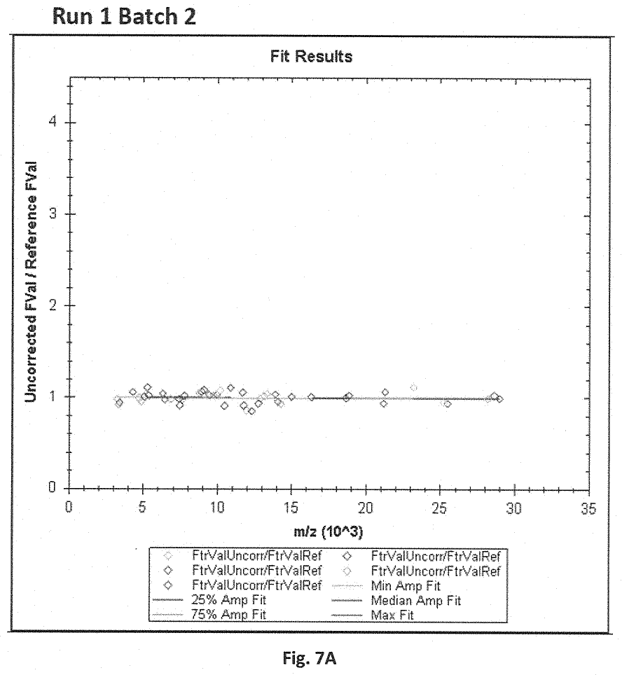

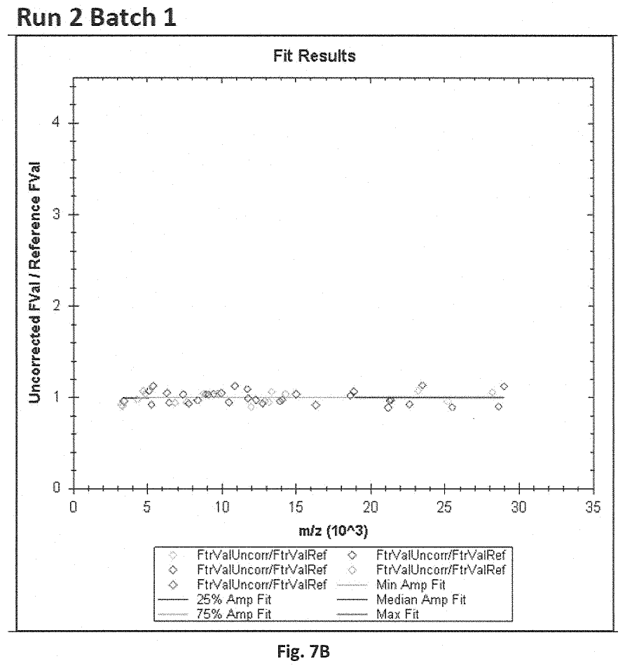

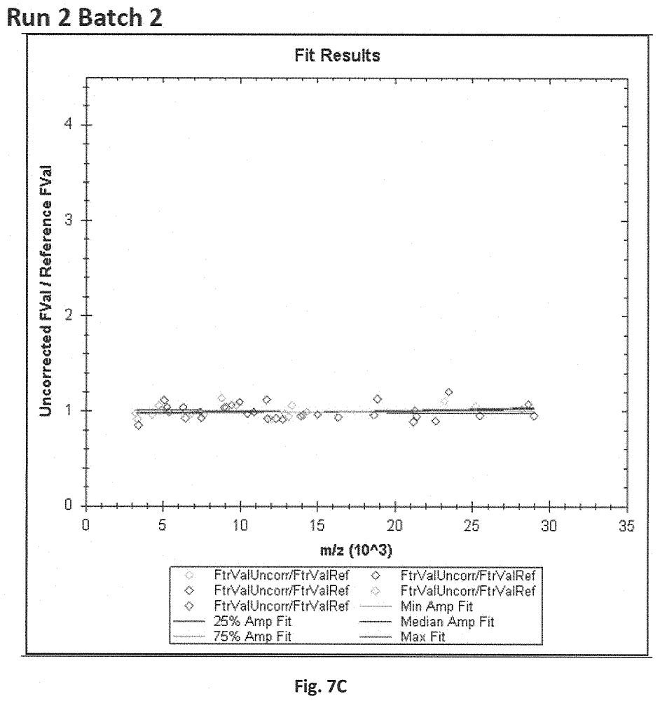

After this correction, the corrected (Āᵢ^{j}, (m/z)ᵢ,\hat{r}ᵢ^{j}) feature values calculated for reference spectra lie around the horizontal line defined by r=1, as shown in FIGS. 6A, 6B, and 6C. Post correction coefficients are calculated to compare to quality control thresholds. These coefficients can be found in Table 19, and the corresponding plots in FIGS. 7A, 7B, and 7C.

TABLE 19

| Model | A0 | A1 | B0 | B1 | C | ResSD |
|---|---|---|---|---|---|---|
| Run 1 Batch 2 | 1.001E+00 | 3.936E−04 | −2.396E−07 | −2.907E−08 | 8.613E−12 | 6.069E−02 |
| Run 2 Batch 1 | 9.994E−01 | −1.384E−04 | 1.193E−07 | 2.268E−08 | −3.765E−12 | 6.819E−02 |
| Run 2 Batch 2 | 9.778E−01 | 1.249E−02 | 7.435E−07 | −8.698E−07 | 4.055E−11 | 7.398E−02 |

Partial Ion Current (PIC) normalization: the dataset was examined to find regions of intrinsic stability to use as the final normalization windows. First, p values comparing the original response groups (CR vs other) were computed. Features with p values less than 0.10 were excluded resulting in 271 features (of 418) to be used in the PIC analysis. As a result of the PIC analysis, 39 features were selected for PIC normalization and these are listed in Table 20.

TABLE 20
Features used for PIC normalization
Feature (m/z)

3238
3393
3722
3773
3815
3905
4011
4286
4916
5067
5101
5190
5288
5323
5475
5520
5553
6030
6192
7259
7299
7473
7739
8366
8478
9263
10922
14591
14642
16646
16930
19090
20815
21580
21854
21906
22357
22388
22604

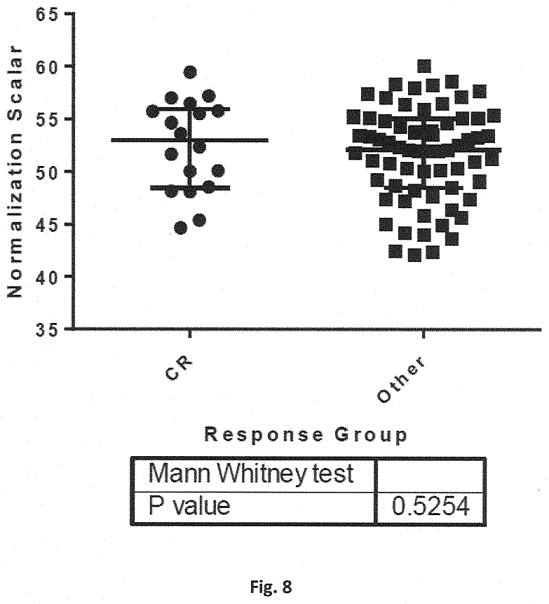

To normalize, the feature values from the listed features were summed for each spectrum to compute a normalization scalar. All feature values were then divided by the normalization scalar per sample to arrive at the final table used in for new classifier development. The normalization scalars were again examined by clinical group to check that the combined features, i.e., the normalization scalars themselves, were not correlated with clinical group. The plot in FIG. 8 illustrates the distribution of the scalars by group. The final feature table, containing all 85 samples in the analysis cohort, was prepared using the PIC normalization features listed above.

### The Diagnostic Cortex™

New classifier development was carried out using the Diagnostic Cortex platform, shown schematically in FIG. 9.

Definition of Class Labels: while some preliminary approaches explored for classifier development employed well-defined class labels, such as response categories, these proved to be unsuccessful in creating classifiers with good performance. All approaches used for purposes of the invention use time-to-event data for classifier training. In this situation class labels are not obvious and, as shown in FIG. 9, the diagnostic cortex uses an iterative method to refine class labels at the same time as creating the classifier. An initial guess is made for the class labels. Typically the samples are sorted on either PFS or OS and half of the samples with the lowest time-to-event outcome are assigned the “Early” class label (early death or progression, i.e., poor outcome) while the other half are assigned the “Late” class label (late death or progression, i.e., good outcome). For the classifiers disclosed herein PFS was used. A classifier is then constructed using the outcome data and these class labels. This classifier can then be used to generate classifications for the development set samples and these are then used as the new class labels for a second iteration of the classifier construction step. This process is iterated until convergence.

Creation and Filtering of Mini-Classifiers: the development set samples are split into training and test sets in multiple different random realizations. Six hundred and twenty five realizations were used. The diagnostic cortex platform works best when training classes have the same number of samples. Hence, if classes have different numbers of members, they are split in different ratios into test and training.

Many k-nearest neighbor (kNN) mini-classifiers (mCs) that use the training set as their reference set are constructed using subsets of features. All classifiers described herein use k=9. The classifiers described herein use only mCs with single features and pairs of features.

To target a final classifier that has certain performance characteristics, the mCs are filtered as follows. Each mC is applied to its training set and performance metrics are calculated from the resulting classifications of the training set. Only mCs that satisfy thresholds on these performance metrics pass filtering to be used further in the process. The mCs that fail filtering are discarded. All classifiers presented in this report used filtering based on hazard ratios. For hazard ratio filtering, the mC is applied to its training set. The hazard ratio for a specified outcome (here PFS) is then calculated between the group classified as Early and the rest classified as Late. The hazard ratio must lie within specified bounds for the mC to pass filtering.

Combination of mini-classifiers using logistic regression with dropout: once the filtering of the mCs is complete, the mCs are combined into one master classifier (MC) using a logistic regression trained on the training set class labels. To help avoid overfitting the regression is regularized using extreme drop out with only a small number of the mCs chosen randomly for inclusion in each of the logistic regression iterations. The number of dropout iterations is selected based on the typical number of mCs passing filtering to ensure that each mC is likely to be included within the drop out process multiple times. Classifiers presented in this report left in 10 randomly selected mCs per drop out iteration and used either 10,000 or 100,000 drop out iterations.

Training/Test splits: the use of multiple training/test splits avoids selection of a single, particularly advantageous or difficult training set for classifier creation and avoids bias in performance assessment from testing on a test set that could be especially easy or difficult to classify.

The output of the logistic regression that defines each MC is a probability of being in one of the two training classes (Early or Late). Applying a threshold to this output produces a binary label (Early or Late) for each MC. For all classifiers presented herein, a cutoff threshold of 0.5 was used. To produce an overall final classification, a majority vote is done across all MCs (“ensemble average”). When classifying samples in the development set this is modified to incorporate in the majority vote only MCs where the sample is not in the training set (“out-of-bag majority vote”).

It is also possible to directly average the MC probabilities to yield one average probability for a sample. When working with the development set, this approach is adjusted to average over MCs for which a given sample is not included in the training set (“out-of-bag” estimate). These average probabilities can then be converted into a binary classification by applying a cutoff. Applying a cutoff of 0.5 to the averaged probabilities gives very similar classifications to using a cutoff of 0.5 on the individual MC probabilities and then performing the majority vote over the MCs. However, this approach was not used to produce the results shown herein.

### Classifiers Developed and Their Performance

Classifier 1/Design: this classifier consists of a hierarchical combination of 2 sub-classifiers, each of them developed using subsets of mass spectral features which have been identified as being associated with the Complement and Acute Response protein functional groups, respectively. This was done using the principles of gene set enrichment analysis (GSEA).

Gene Set Enrichment Analysis (GSEA) is a method frequently used in gene expression analysis studies when expression values for a large number of genes are available for a number of biological samples for which either categorical class information or the value of some continuous variable is also known [Mootha et al., Nat Genet. 2003; 34(3):267-73; Subramanian et al., Proc Natl Acad Sci USA 2005; 102(43): 15545-50]. The approach looks for a pattern of correlations of gene expression of the samples with the associated categorical or continuous variable depending on the biological function of the genes. The approach was developed for use in gene expression studies, but it can be equally well applied to protein expression data, and this is the context in which it will be discussed here.

The general approach is to rank the entire list of measured proteins according to their correlation with a categorical label or continuous variable, from highest to lowest. Subsets of proteins from the universe of measured proteins are defined based on their biological functions, e.g., using well-known databases such as UniProt or GeneOntology/AmiGO2. The method then looks for over- or under-representation of the proteins in each subset as a function of rank in the ranked list of all measured proteins. The method implemented herein follows the approach of Subramanian. No corrections are made for multiple comparisons.

A cohort of 49 serum samples is available with matched protein expression data and deep MALDI spectra. The protein expression data comes from running the SomaLogic 1129 protein panel on the serum samples. Any mass spectral feature values or test classifications can be generated on this spectra; data and correlated with the protein expression data. Investigations used 29 different protein sets defined as the intersection of the results of querying protein databases on specific biological process and the list of 1129 measured proteins. Protein sets were selected to include functions expected to play a role in the immune system and cancer treatment efficacy in general, as well as others not expected to be relevant, as a control. There is overlap between some of the protein sets, as would be expected from the similar biological keywords used in their construction.

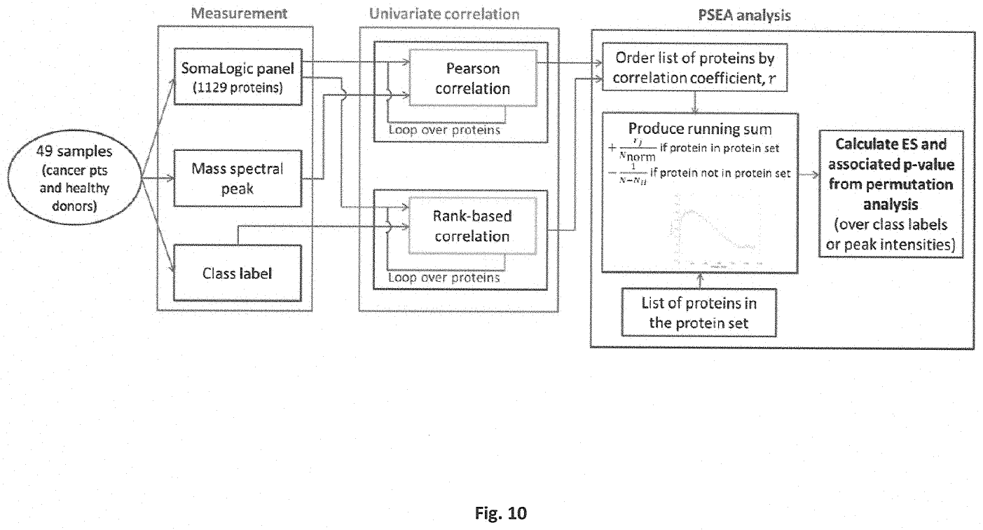

GSEA method for association of mass spectral features with protein functional groups: for this application the correlation of protein expression data with mass spectral feature values is investigated, i.e., the continuous variable used in GSEA is a mass spectral feature value. The GSEA method was applied for each of the 418 mass spectral features. Features with a p<0.05 for the GSEA for a particular protein functional set were designated as associated with that biological function. This is illustrated schematically in FIG. 10. In this way, subsets of the 418 mass spectral features were generated associated with each of the tested protein functional sets. For example, it was determined that 37 mass spectral features were associated with acute response and 142 with complement activation. These subsets of features were used in the creation of Classifier 1.

GSEA method for association of test classifications with protein functional groups: for this application, a developed test (Classifier 2) is applied to the deep MALDI spectra acquired from the 49 sample cohort and test classifications are generated which are then correlated with the protein expression data. This method was used to assess what biological functions may be associated with test classifications.

The first sub-classifier was designed using 83 of the 85 samples in the analysis cohort as the development set. Spectra from two patients not evaluable for response were not included in the training of this sub-classifier. The subset of 142 mass spectral features associated with complement activation and with m/z<25 kDa were used in the Diagnostic Cortex platform to create a classifier able to stratify patients into two groups with better and worse PFS. No feature deselection was used, i.e., all 142 mass spectral features associated with complement were used at each step of refinement of the class labels and first sub-classifier. Twenty-nine samples of the analysis cohort were assigned to the poor performing group and these were given an “Early” classification. The remaining 56 samples, assigned to the good performing group, were used as the development set for a second sub-classifier. This sub-classifier was trained on the subset of 37 mass spectral features which had been identified as being associated with acute response (AR). The second classifier again used no feature deselection and stratified patients well into groups with better or worse PFS. Samples in the good outcome group were assigned a “Late” classification and samples in the poor outcome group were assigned an “Early” classification. The feature subsets used in the creation of the first sub-classifier and the second sub-classifier are given in Table 21. In some embodiments, for each respective feature given in Table 21, the corresponding m/z range given in Table 16 was used to calculate the feature value for the respective feature. For example, for the feature “3125” listed in Table 21 for sub-classifier 1, a mass spectrograph of a sample from a target entity was integrated between 3118.81 (m/z) and 3130.38 (m/z) as specified in Table 16 (entry number 3: 3118.81, 3124.60, 3130.38) in order to arrive at the feature value for this feature. This feature value was then used in pattern classification techniques as discussed herein in order to classify a target entity.

TABLE 21
Features used in Classifier 1 Schema

| Sub-classifier 1 | Sub-classifier 2 |
|---|---|
| (complement) | (acute response) |
| Column 1 | Column 2 |
| 3125 | 3611 |
| 3138 | 3702 |
| 3214 | 3839 |
| 3238 | 4534 |
| 3261 | 4585 |
| 3312 | 4916 |
| 3516 | 5249 |
| 3532 | 5288 |
| 3588 | 5392 |
| 3702 | 5553 |
| 3722 | 5635 |
| 3738 | 5721 |
| 3753 | 5987 |
| 3773 | 6076 |
| 3839 | 6880 |
| 3890 | 6898 |
| 3932 | 6920 |
| 4030 | 6986 |
| 4074 | 7005 |
| 4340 | 7244 |
| 4379 | 7473 |
| 4460 | 7912 |
| 4585 | 8153 |
| 4710 | 8509 |
| 4755 | 9869 |
| 4818 | 11786 |
| 4856 | 11843 |
| 4937 | 12414 |
| 4962 | 12736 |
| 5019 | 13570 |
| 5288 | 13766 |
| 5504 | 13981 |
| 5520 | 18269 |
| 5553 | 18628 |
| 5691 | 18843 |
| 5721 | 22388 |
| 5736 | 23031 |
| 5763 | |
| 5815 | |
| 5841 | |
| 5869 | |
| 5891 | |
| 5922 | |
| 5945 | |
| 6007 | |
| 6076 | |
| 6089 | |
| 6149 | |
| 6172 | |
| 6211 | |
| 6241 | |
| 6432 | |
| 6455 | |
| 6633 | |
| 6807 | |
| 6837 | |
| 6859 | |
| 6880 | |
| 6939 | |
| 6969 | |
| 6986 | |
| 7042 | |
| 7142 | |
| 7359 | |
| 7419 | |
| 7473 | |
| 8490 | |
| 8564 | |
| 8591 | |
| 8768 | |
| 8892 | |
| 8926 | |
| 9064 | |
| 9097 | |
| 9133 | |
| 9287 | |
| 9359 | |
| 9427 | |
| 9468 | |
| 9505 | |
| 9584 | |
| 9641 | |
| 9719 | |
| 9788 | |
| 9869 | |
| 9919 | |
| 10138 | |
| 10212 | |
| 10637 | |
| 11003 | |
| 11150 | |
| 11393 | |
| 11445 | |
| 11480 | |
| 11530 | |
| 11577 | |
| 11630 | |
| 11687 | |
| 11745 | |
| 11786 | |
| 11843 | |
| 11899 | |
| 11998 | |
| 12116 | |
| 12210 | |
| 12290 | |
| 12319 | |
| 12414 | |
| 12736 | |
| 13134 | |
| 13319 | |
| 13364 | |
| 13510 | |
| 13612 | |
| 13722 | |
| 13766 | |
| 13886 | |
| 13943 | |
| 13981 | |
| 14098 | |
| 14304 | |
| 14433 | |
| 14539 | |
| 14642 | |
| 14670 | |
| 14783 | |
| 18269 | |
| 18628 | |
| 18727 | |
| 18843 | |
| 19090 | |
| 20950 | |
| 21061 | |
| 21165 | |
| 21265 | |
| 23031 | |
| 23130 | |
| 23243 | |
| 23352 | |
| 23463 | |
| 23562 | |
| 23666 | |

Samples classified by the first sub-classifier (based on complement-associated mass spectral (MS) features) as belonging to the poor performing group were given the “Bad” final classification. Those assigned to the good performing group by the first sub-classifier were given a classification of “Good” if the second sub-classifier (based on acute response-related MS features) gave a classification of “Late”, and a classification of “Intermediate” if the second sub-classifier gave a classification of “Early.”

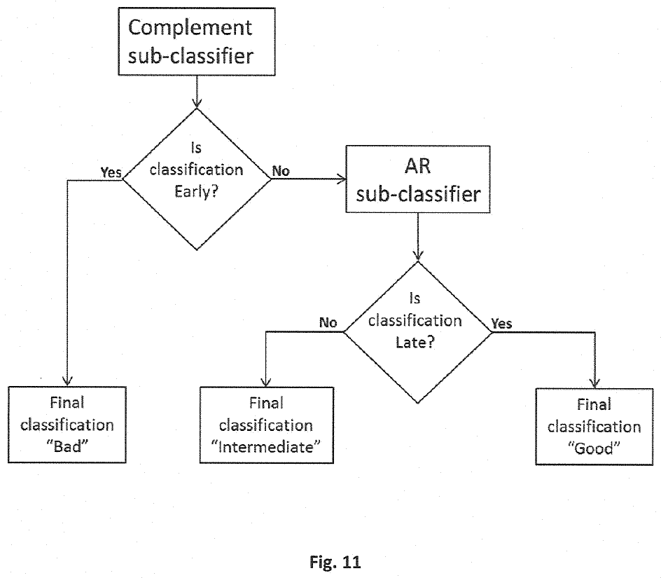

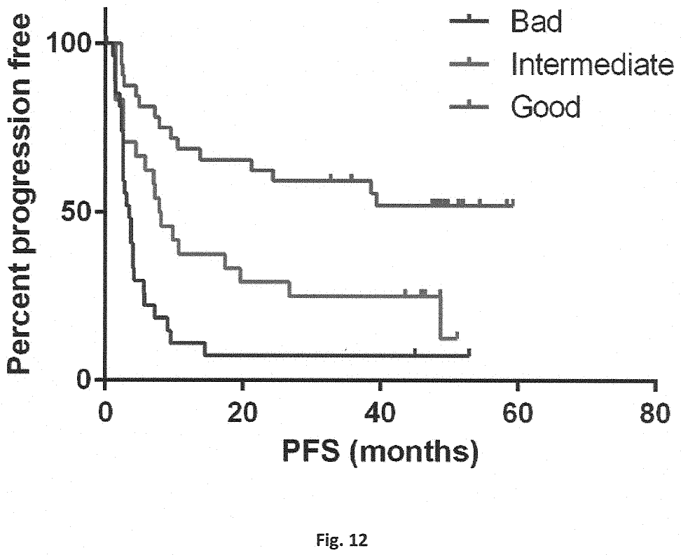

Results: the developed classifier assigned 29 Bad classifications (34%), 24 Intermediate classifications (28%), and 32 Good classifications (38%). Classifications by sample are given in Table 22. Baseline characteristics by test classification are summarized in Table 23 and response to therapy also split by test classification is shown in Table 24. Kaplan-Meier plots of PFS split by test classification are shown in FIG. 11 and a performance summary is presented in Table 25.

TABLE 22
TEST CLASSIFICATIONS BY SAMPLE

| Sample ID | Classifier 1 | Classifier 2 |
|---|---|---|
| HARV_SP_0594_002 | Good | Good |
| HARV_SP_0616_002 | Intermediate | Bad |
| HARV_SP_0651_002 | Bad | Bad |
| HARV_SP_0653_002 | Bad | Bad |
| HARV_SP_0982_002 | Good | Good |
| HARV_SP_1418_001 | Intermediate | Good |
| HARV_SP_1487_002 | Bad | Bad |
| HARV_SP_1490_002 | Good | Good |
| HARV_SP_1506_002 | Good | Good |
| HARV_SP_1592_002 | Intermediate | Bad |
| HARV_SP_1709_001 | Bad | Bad |
| HARV_SP_1762_001 | Good | Good |
| HARV_SP_1979_002 | Good | Good |
| HARV_SP_2557_002 | Good | Good |
| HARV_SP_2876_001 | Bad | Bad |
| HARV_SP_3065_001 | Intermediate | Bad |
| NV12_SP_0096_002 | Bad | Bad |
| NV12_SP_0109_002 | Bad | Bad |
| NV12_SP_0142_002 | Good | Good |
| NV12_SP_0157_002 | Bad | Bad |
| NV12_SP_0162_002 | Intermediate | Bad |
| NV12_SP_0218_002 | Good | Good |
| NV12_SP_0238_002 | Intermediate | Bad |
| NV12_SP_0252_002 | Bad | Bad |
| NV12_SP_0257_002 | Bad | Bad |
| NV12_SP_0278_002 | Good | Good |
| NV12_SP_0370_002 | Intermediate | Bad |
| NV12_SP_0382_002 | Good | Good |
| NV12_SP_0401_002 | Intermediate | Bad |
| NV12_SP_0429_002 | Good | Good |
| NV12_SP_0492_001 | Intermediate | Bad |
| NV12_SP_0495_002 | Intermediate | Good |
| NV12_SP_0572_002 | Bad | Bad |
| NV12_SP_0595_002 | Intermediate | Bad |
| NV12_SP_0597_002 | Good | Good |
| NV12_SP_0640_002 | Bad | Bad |
| NV12_SP_0745_001 | Good | Good |
| NV12_SP_0754_002 | Good | Good |
| NV12_SP_0768_002 | Good | Good |
| NV12_SP_0792_002 | Intermediate | Good |
| NV12_SP_0841_002 | Good | Good |
| NV12_SP_0872_001 | Good | Good |
| NV12_SP_0935_002 | Intermediate | Bad |
| NV12_SP_0961_002 | Good | Good |
| NV12_SP_1014_002 | Intermediate | Good |
| NV12_SP_1034_002 | Intermediate | Bad |
| NV12_SP_1097_002 | Bad | Bad |
| NV12_SP_1104_002 | Bad | Bad |
| NV12_SP_1109_002 | Intermediate | Good |
| NV12_SP_1132_002 | Intermediate | Bad |
| NV12_SP_1223_002 | Good | Good |
| NV12_SP_1249_002 | Bad | Bad |
| NV12_SP_1268_002 | Intermediate | Good |
| NV12_SP_1333_002 | Good | Good |
| NV12_SP_1335_002 | Bad | Bad |
| NV12_SP_1354_002 | Bad | Bad |
| NV12_SP_1419_002 | Bad | Bad |
| NV12_SP_1433_002 | Intermediate | Bad |
| NV12_SP_1434_002 | Bad | Bad |
| NV12_SP_1454_002 | Intermediate | Bad |
| NV12_SP_1495_002 | Good | Good |
| NV12_SP_1534_002 | Good | Good |
| NV12_SP_1562_001 | Good | Good |
| NV12_SP_1563_002 | Intermediate | Good |
| NV12_SP_1566_001 | Bad | Bad |
| NV12_SP_1637_001 | Intermediate | Good |
| NV12_SP_1639_002 | Good | Good |
| NV12_SP_1683_002 | Good | Good |
| NV12_SP_1703_002 | Bad | Bad |
| NV12_SP_1726_001 | Bad | Bad |
| NV12_SP_1732_001 | Good | Good |
| NV12_SP_1767_002 | Intermediate | Good |
| NV12_SP_1790_002 | Bad | Bad |
| NV12_SP_1830_001 | Bad | Bad |
| NV12_SP_1857_002 | Good | Good |
| NV12_SP_1891_002 | Bad | Bad |
| NV12_SP_1892_002 | Good | Good |
| NV12_SP_1934_002 | Good | Good |
| NV12_SP_1936_002 | Bad | Bad |
| NV12_SP_1996_001 | Bad | Bad |
| NV12_SP_2003_002 | Bad | Bad |
| NV12_SP_2062_001 | Good | Good |
| NV12_SP_2078_002 | Good | Good |
| NV12_SP_2144_001 | Bad | Bad |
| NV12_SP_2189_001 | Intermediate | Bad |

TABLE 23
Baseline clinical characteristics by Classifier 1 classification
of the analysis cohort of 85 patients

| | | Bad | | Intermediate | | Good | |
|---|---|---|---|---|---|---|---|
| | | (N = 29) | | (N = 24) | | (N = 32) | |
| | | n (%) | | n (%) | | n (%) | |
| Age, median (Range) | | 47 | (21-62) | 47 | (20-65) | 45 | (30-60) |
| Gender | Male | 18 | (62) | 15 | (63) | 22 | (69) |
| | Female | 11 | (38) | 9 | (38) | 10 | (31) |
| Prior Adjuvant | Yes | 11 | (38) | 8 | (33) | 14 | (44) |
| Therapy | No | 15 | (52) | 14 | (58) | 14 | (44) |
| | N/A | 3 | (10) | 2 | (8) | 4 | (13) |
| Treatment Line | 1 | 7 | (24) | 10 | (42) | 12 | (38) |
| | 2 | 7 | (24) | 3 | (13) | 8 | (25) |
| | 3 | 6 | (21) | 5 | (21) | 5 | (16) |
| | 4 | 4 | (14) | 2 | (8) | 3 | (9) |
| | 5 | 2 | (7) | 2 | (8) | 0 | (0) |
| | N/A | 3 | (10) | 2 | (8) | 4 | (13) |
| Race | White | 29 | (90) | 23 | (96) | 31 | (97) |
| | Black | 1 | (3) | 1 | (4) | 0 | (0) |
| | Asian | 0 | (0) | 0 | (0) | 1 | (3) |
| | N/A | 2 | (7) | 0 | (0) | 0 | (0) |

TABLE 24
Response to therapy by Classifier 1 classification
for the analysis cohort of 85 patients

| | Bad | | Intermediate | | Good | |
|---|---|---|---|---|---|---|
| | (N = 29) | | (N = 24) | | (N = 32) | |
| Response | n (%) | | n (%) | | n (%) | |
| CR | 3 | (10) | 2 | (8) | 13 | (41) |
| Progression before 1 yr | 0 | | 0 | | 0 | |
| Progression before 2 yrs | 1 | | 0 | | 0 | |
| Progression before 3 yrs | 1 | | 0 | | 0 | |
| Progression before 4 yrs | 1 | | 0 | | 2 | |
| PR | 6 | (21) | 11 | (46) | 11 | (34) |
| Progression before 1 yr | 6 | | 6 | | 3 | |
| Progression before 2 yrs | 6 | | 8 | | 5 | |
| Progression before 3 yrs | 6 | | 8 | | 6 | |
| SD | 0 | (0) | 1 | (4) | 1 | (3) |
| PD | 18 | (62) | 10 | (42) | 7 | (22) |
| NE | 2 | (7) | 0 | (0) | 0 | (0) |

TABLE 25
Performance statistics for Classifier 1

| | PFS HR | PFS | PFS Median | % progression- | % progression- |
|---|---|---|---|---|---|
| | (95% CI) | log-rank p | (months) | free at 2 years | free at 4 years |
| Good vs Bad | 0.24 | <0.001 | Bad: 3.5 | Bad: 7% | Bad: 7% |
| | (0.08-0.31) | | Good: not reached | Good: 63% | Good: 52% |
| Good vs | 0.42 | 0.008 | Intermediate: 8.0 | Intermediate: 29% | Intermediate: 25% |
| Intermediate | (0.19-0.76) | | Good: not reached | Good: 63% | Good: 52% |
| Intermediate | 0.50 | 0.014 | Bad: 3.5 | Bad: 7% | Bad: 7% |
| vs Bad | (0.25-0.83) | | Intermediate: 8.0 | Intermediate: 29% | Intermediate: 25% |

Reproducibility: a rerun of all 85 analysis samples was performed. This was carried out with completely independent sample preparation and spectral acquisition and processing after a second machine qualification run. The generated spectra were analyzed and classifications compared with those of the initial run to evaluate the reproducibility of the test. Table 26 shows the test classification concordance between the two runs. The overall concordance is 76/85=89%.

TABLE 26
Classifier 1 concordance between run 1 and run 2

Original run - run 1
(development)

| | | Bad | Intermediate | Good |
|---|---|---|---|---|
| | | (n = 29) | (n = 24) | (n = 32) |
| Rerun - | Bad (n = 28) | 28 | 0 | 0 |
| run 2 | Intermediate | 1 | 19 | 3 |
| | (n = 20) | | | |
| | Good (n = 34) | 0 | 5 | 29 |

Classifier 2/Design: this classifier consists of the combination of the 2 sub-classifiers of classifier 1 and an existing third sub-classifier from a previously developed test (“IS13”). This pre-existing test was constructed using melanoma samples with the goal of identifying patients with durable benefit from immunotherapies in poor prognosis groups and assigns the classifications of EarlyEarly or EarlyLate (worse or better outcome on immunotherapy).

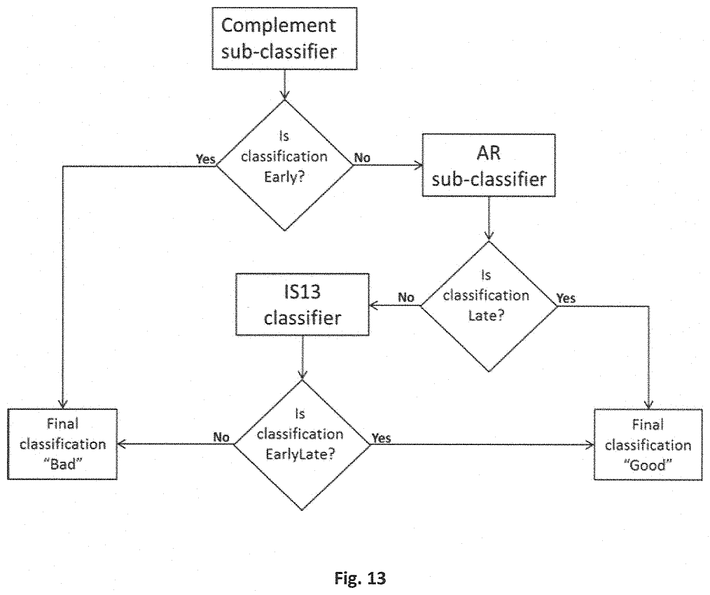

Samples classified by the first sub-classifier (based on complement MS features) as belonging to the poor performing group were given the “Bad” final classification. Those samples assigned to the good performing group both by the first and second sub-classifiers were given a classification of “Good”. The classification of the remaining samples, assigned to the good performing group by the first sub-classifier and to the poor performing group by the second sub-classifier, was based on the classification given by the third sub-classifier: if the classification was EarlyEarly the final classification was Bad and if the classification was EarlyLate the final classification was Good. This procedure for assigning classifications is summarized in FIG. 13.

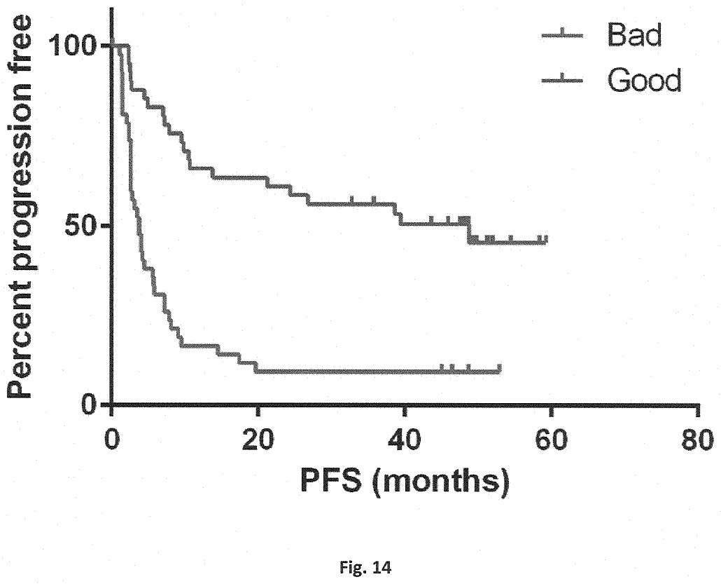

Results: classifier 2 assigned 44 Bad classifications (52%) and 41 Good classifications (48%). Classifications by sample are listed in Table 22. Baseline characteristics by test classification are summarized in Table 27, and response to therapy also split by test classification is shown in Table 28. Kaplan-Meier plots of PFS split by test classification are shown in FIG. 14 and a performance summary is presented in Table 29.

TABLE 27
Baseline clinical characteristics by Classifier 2 classification
of the analysis cohort of 85 patients

| | | Bad | | Good | |
|---|---|---|---|---|---|
| | | (N = 44) | | (N = 41) | |
| | | n (%) | | n (%) | |
| Age, median (Range) | | 47 | (21-62) | 46 | (20-60) |
| Gender | Male | 28 | (64) | 27 | (66) |
| | Female | 16 | (36) | 14 | (34) |
| Prior Adjuvant | Yes | 17 | (39) | 16 | (39) |
| Therapy | No | 24 | (55) | 19 | (46) |
| | N/A | 3 | (7) | 6 | (15) |
| Treatment Line | 1 | 12 | (27) | 17 | (41) |
| | 2 | 9 | (20) | 9 | (22) |
| | 3 | 11 | (25) | 5 | (12) |
| | 4 | 5 | (11) | 4 | (10) |
| | 5 | 4 | (9) | 0 | (0) |
| | N/A | 3 | (7) | 6 | (15) |
| Race | White | 40 | (91) | 40 | (98) |
| | Black | 2 | (5) | 0 | (0) |
| | Asian | 0 | (0) | 1 | (2) |
| | N/A | 2 | (5) | 0 | (0) |

TABLE 28
Response to therapy by Classifier 2 classification
for the analysis cohort of 85 patients

| | | |
|---|---|---|
| | Bad | Good |
| | (N = 44) | (N = 41) |
| Response | n (%) | n (%) |

| CR | 4 | (9) | 14 | (34) |
|---|---|---|---|---|
| Progression before 1 yr | 0 | | 0 | |
| Progression before 2 yrs | 1 | | 0 | |
| Progression before 3 yrs | 1 | | 0 | |
| Progression before 4 yrs | 1 | | 2 | |
| PR | 13 | (30) | 15 | (37) |
| Progression before 1 yr | 10 | | 5 | |
| Progression before 2 yrs | 12 | | 7 | |
| Progression before 3 yrs | 12 | | 8 | |
| SD | 0 | (0) | 2 | (5) |
| PD | 25 | (57) | 10 | (24) |
| NE | 2 | (5) | 0 | (0) |

TABLE 29
Summary of the performance of Classifier 2 on the analysis cohort

| | | PFS | | |
|---|---|---|---|---|
| PFS HR | PFS log- | Median | % progression- | % progression- |
| (95% CI) | rank p | (months) | free at 2 years | free at 4 years |
| 0.28 | <0.001 | Bad: 3.7 | Bad: 10% | Bad: 10% |
| (0.13-0.37) | | Good: 48.7 | Good: 61% | Good: 50% |

Reproducibility: a rerun of all 85 analysis samples was performed. This was carried out with completely independent sample preparation and spectral acquisition and processing after a second machine qualification run. The generated spectra were analyzed and classifications compared with those of the initial run to evaluate the reproducibility of the test. Table 30 shows the test classification concordance between the two runs. The overall concordance is 78/85=92%.

TABLE 30
Classifier 2 concordance between run 1 and run 2

Original run - run 1
(development)

| | | Bad (n = 44) | Good (n = 41) |
|---|---|---|---|
| Rerun - | Bad (n = 39) | 39 | 2 |
| run 2 | Good (n = 43) | 5 | 39 |

### Relation to Protein Functional Groups

Protein Set Enrichment Analysis (PSEA), a method inspired by gene set enrichment analysis, was used to look for an association of the test classifications (Classifier 2) with protein functional groups. To do this, an independent set of 49 samples was used where paired deep MALDI spectra and protein panel (Somalogic, Boulder, Colo.) results were available. Of the 49 samples 35 classified as Bad and 14 as Good.

The results for 29 different protein functional groups tested are shown in Table 31. P values are not corrected for multiple comparisons. At the α=0.05 significance level, associations of the test classifications were found with acute inflammation, complement, acute response and acute phase. In addition, at the α=0.10 significance level, associations of the test classifications were found with glycolytic processes and extracellular matrix.

TABLE 31
Results of Protein Set Enrichment Analysis for Classifier 2

| Protein Set Description | Enrichment score | p value |
|---|---|---|
| Acute inflammation | 0.399 | 0.037 |
| Innate Immune Response | 0.495 | 0.359 |
| Adaptive immune response | 0.335 | 0.561 |
| Gh colytic Process | −0.642 | 0.068 |
| Immune T-cells | −0.220 | 0.658 |
| Immune B-cells | −0.223 | 0.881 |
| Cell cycle | −0.287 | 0.279 |
| NK regulation | −0.376 | 0.471 |
| Complement | 0.564 | 0.008 |
| Cancer - experimental | 0.839 | 0.318 |
| Acute response | 0.580 | 0.049 |
| Cytokine activity | −0.221 | 0.735 |
| Wound healing | −0.343 | 0.178 |
| Interferon | −0.227 | 0.690 |
| Interleukin-10 | 0.181 | 0.839 |
| GFR* signaling | −0.191 | 0.749 |
| Immune response | 0.261 | 0.116 |
| Immune Response Type 1 | 0.319 | 0.805 |
| Immune Response Type 2 | −0.279 | 0.968 |
| Immune Response - Complement | −0.219 | 0.400 |
| Immune Response - Complement - | −0.263 | 0.127 |
| Acute | | |
| Acute phase | 0.525 | 0.032 |
| Hypoxia | −0.286 | 0.437 |
| Cancer | 0.174 | 0.787 |
| Cell adhesion | −0.176 | 0.876 |
| Mesenchymal transition | 0.304 | 0.773 |
| Extracellular matrix - restricted | −0.467 | 0.064 |
| source, UNIPROT | | |
| Extracellular matrix - from | −0.372 | 0.082 |
| different sources | | |
| Angiogenesis | −0.245 | 0.480 |

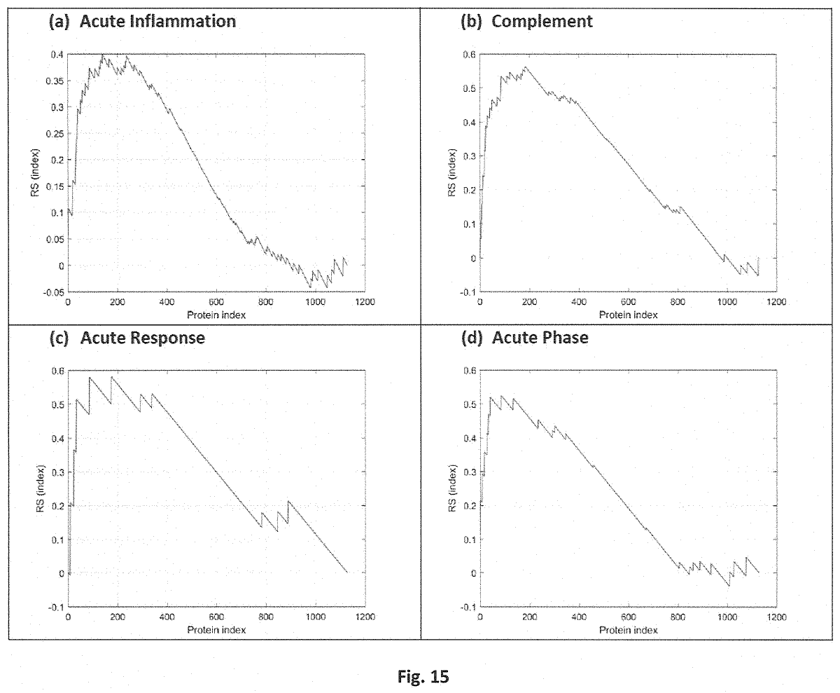

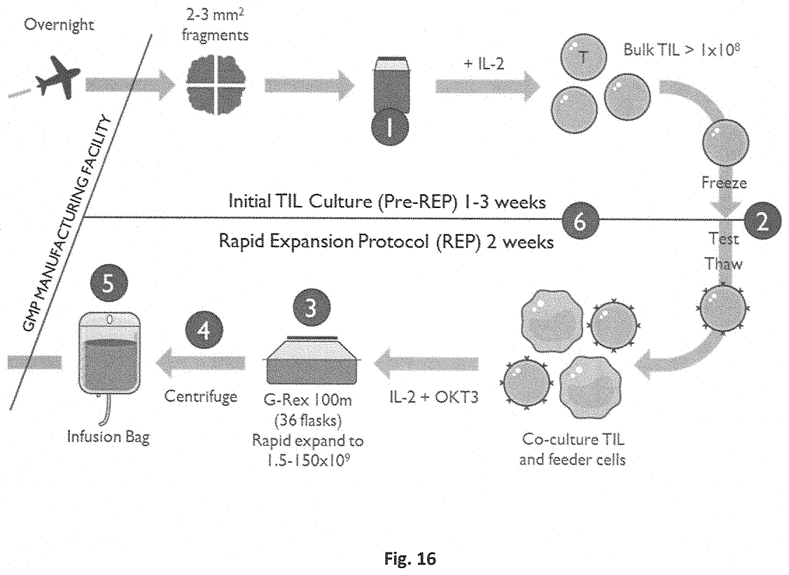

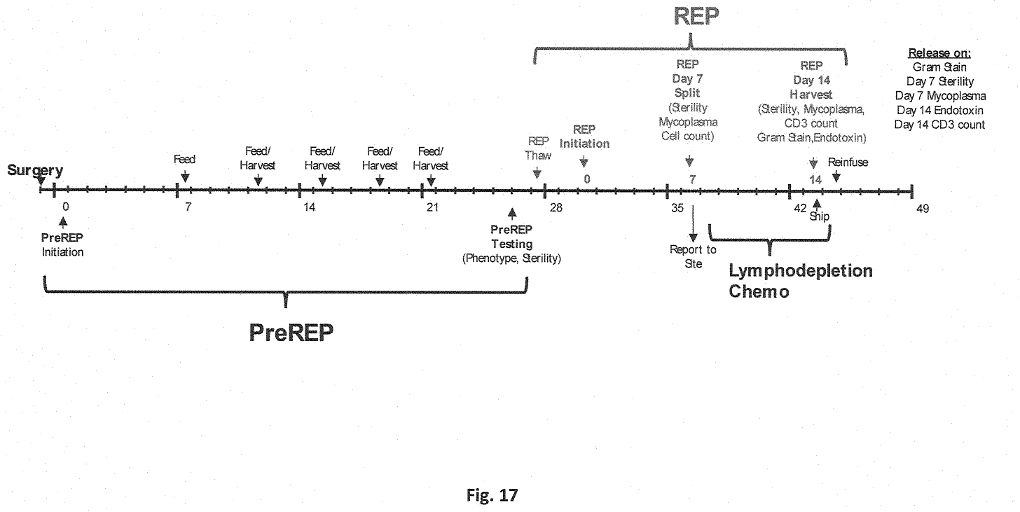

Plots of the running sum, RS(i), produced during PSEA are shown in FIG. 15 for acute inflammation, complement, acute response and acute phase [Subramanian et al., Proc Natl Acad Sci USA 2005; 102(43): 15545], suggest an approach of examining the subset of the protein set (associated with a relevant biological function) that comprises the “leading edge” of the RS plot, i.e., the subset of the protein set that contributes to the increase in RS up to its maximum deviation from the x axis. Both proteins from the set that are either highly correlated or highly anti-correlated with classifier labels (Bad and Good) were included. In addition to the proteins included up the leading edge, the correlation of the protein at the maximum deviation is found and proteins that have greater absolute correlation but opposite sign are also included in an “extended leading edge” set. The extended leading edge sets are listed for the four protein sets shown in FIG. 15 and Tables 32-35. The correlations given in Tables 32-35 are a scaled version of the rank sum statistic so that 1 represents perfect correlation, −1 perfect anti-correlation, and 0 no correlation.

TABLE 32
Proteins included in the extended leading
edge set of acute inflammation.

| UniProt | | | |
|---|---|---|---|
| ID | Protein Name | Correlation | P value |
| P01009 | alpha1-Antitrypsin | 0.743 | <0.001 |
| P02741 | C-reactive protein | 0.682 | <0.001 |
| P01024 | Complement C3a anaphylatoxin | 0.461 | 0.012 |
| P02679 | Fibrinogen gamma chain dimer | 0.457 | 0.013 |
| P01024 | Complement C3 | 0.412 | 0.025 |
| Q14624 | Inter-alpha-trypsin inhibitor | 0.408 | 0.027 |
| | heavy chain H4 | | |
| Q8NEV9 | Interleukin-27 | 0.396 | 0.032 |
| P07951 | Tropomyosin beta chain | 0.388 | 0.036 |
| P02743 | Serum amyloid P | 0.376 | 0.042 |
| Q00535 | Cyclin-dependent kinase 5: | 0.347 | 0.060 |
| | activator p35 complex | | |
| P33681 | T-lymphocyte activation antigen | 0.327 | 0.077 |
| | CD80 | | |
| P05156 | Complement factor I | 0.306 | 0.097 |
| P11226 | Mannose-binding protein C | 0.290 | 0.116 |
| P47710 | alpha-S1-casein | 0.286 | 0.121 |
| P07357 | Complement C8 | 0.253 | 0.170 |
| P27797 | Calreticulin | 0.241 | 0.192 |
| P00738 | Haptoglobin | 0.233 | 0.207 |
| Q9Y5Y7 | Lymphatic vessel endothelial | 0.224 | 0.224 |
| | hyaluronic acid receptor 1 | | |
| P10636 | Microtubule-associated protein tau | −0.253*† | 0.170 |
| P02745 | Complement C1q | −0.261*† | 0.157 |
| P08887 | Interleukin-6 receptor alpha chain | −0.286*† | 0.121 |
| P38919 | Eukaryotic translation initiation | −0.327*† | 0.077 |
| | factor 4A-III | | |
| P08514 | Integrin alpha-IIb: beta-3 complex | −0.351*† | 0.057 |
| P08697 | alpha2-Antiplasmin | −0.376*† | 0.042 |
| P02649 | Apolipoprotein E | −0.481*† | 0.009 |

*indicates proteins to the right of the minimum of RS and
†indicates proteins with anti-correlations of at least as great magnitude as that at the maximum of RS

TABLE 33
Proteins included in the extended leading edge set of complement.

| UniProt | | | |
|---|---|---|---|
| ID | Protein Name | Correlation | P value |
| P02741 | C-reactive protein | 0.682 | <0.001 |
| P01024 | Complement C3b | 0.657 | <0.001 |
| P01024 | Complement C3b, inactivated | 0.563 | 0.002 |
| P0C0L5 | Complement C4b | 0.522 | 0.005 |
| P02748 | Complement C9 | 0.498 | 0.007 |
| P01024 | Complement C3a anaphylatoxin | 0.461 | 0.012 |
| P00751 | Complement factor B | 0.461 | 0.012 |
| P05155 | C1-Esterase Inhibitor | 0.441 | 0.017 |
| P00736 | Complement C1r | 0.437 | 0.018 |
| P01024 | Complement C3 | 0.412 | 0.025 |
| P02743 | Serum amyloid P | 0.376 | 0.042 |
| P06681 | Complement C2 | 0.359 | 0.051 |
| P05156 | Complement factor I | 0.306 | 0.097 |
| P11226 | Mannose-binding protein C | 0.290 | 0.116 |
| Q07021 | Complement C1q subcomponent- | 0.290 | 0.116 |
| | binding protein, mitochondrial | | |
| P01031 | Complement C5a | 0.286 | 0.121 |
| P07357 | Complement C8 | 0.253 | 0.170 |
| P09871 | Complement C1s | 0.245 | 0.184 |
| P01031 | Complement C5b, 6 Complex | 0.216 | 0.241 |
| P12956 | ATP-dependent DNA helicase II | 0.210 | 0.254 |
| | 70 kDa subunit | | |
| P48740 | Mannan-binding lectin serine | 0.208 | 0.259 |
| | peptidase 1 | | |
| P13671 | Complement C6 | 0.200 | 0.278 |
| P16109 | P-Selectin | −0.645*† | <0.001 |
| O75636 | Ficolin-3 | −0.386† | 0.036 |
| P27658 | Collagen alpha-1(VIII) chain | −0.331† | 0.073 |

*indicates proteins to the right of the minimum of RS and
†indicates proteins with anti-correlations of at least as great magnitude as that at the maximum of RS

TABLE 34
Proteins included in the extended
leading edge set of acute response

| UniProt | | | |
|---|---|---|---|
| ID | Protein Name | Correlation | P value |
| P18428 | Lipopolysaccharide-binding | 0.563 | 0.002 |
| | protein | | |
| P05155 | C1-Esterase Inhibitor | 0.441 | 0.017 |
| Q14624 | Inter-alpha-trypsin inhibitor | 0.408 | 0.027 |
| | heavy chain H4 | | |
| Q07021 | Complement C1q subcomponent- | 0.290 | 0.116 |
| | binding protein, mitochondrial | | |
| P48740 | Mannan-binding lectin serine | 0.208 | 0.259 |
| | peptidase 1 | | |

TABLE 35
Proteins included in the extended leading edge set of acute phase.

| UniProt | | | |
|---|---|---|---|
| ID | Protein Name | Correlation | P value |
| P01009 | alpha1-Antitrypsin | 0.743 | <0.001 |
| P02741 | C-reactive protein | 0.682 | <0.001 |
| P18428 | Lipopolysaccharide-binding protein | 0.563 | 0.002 |
| P02671 | D-dimer | 0.473 | 0.010 |
| P0DJI8 | Serum amyloid A | 0.420 | 0.023 |
| Q14624 | Inter-alpha-trypsin inhibitor | 0.408 | 0.027 |
| | heavy chain H4 | | |
| P02743 | Serum amyloid P | 0.376 | 0.042 |
| P11226 | Mannose-binding protein C | 0.290 | 0.116 |
| P08697 | alpha2-Antiplasmin | −0.376*† | 0.042 |
| P02787 | Transferrin | −0.306*† | 0.097 |
| P08887 | Interleukin-6 receptor alpha chain | −0.2861† | 0.121 |

*indicates proteins to the right of the minimum of RS and
†indicates proteins with anti-correlations of at least as great magnitude as that at the maximum of RS

### CONCLUSIONS

Both BDX008 and IL2 tests were able to stratify patients receiving adoptive cell transfer therapy into two groups with better and worse progression-free survival. BDX008 identified a group of approximately one third of patients with particularly poor outcomes (2 year PFS of 7%). The IL2 test identified a group of around one third of patients with particularly good outcomes (4 year PFS of 49%).

New classifier development was able to produce two new tests specifically tailored to the adoptive cell transfer application. Classifier 1 split the analysis cohort into three groups with poor, intermediate and good outcomes. The best performing group, containing 38% of patients, had four year PFS of 52% and a response rate (CR+PR) of 75%. Classifier 2 integrated classifier 1 with an existing Biodesix classifier to stratify patients into two roughly equal sized groups with better and worse outcomes. The good performing group had four year PFS of 50%, a response rate of 71%, and also included the two patients who experienced stable disease in excess of four years. Validation of these new tests can be performed in independent patient cohorts.

Appendix: test classifications are provided for 16 plasma samples collected before adoptive cell transfer (Table 36).

TABLE 36

| | IL2 Test | Classifier 1 | Classifier 2 |
|---|---|---|---|
| Sample ID | Classification | Classification | Classification |
| 15310197_55 | Early | Intermediate | Bad |
| 15336362_55 | Late | Intermediate | Bad |
| 15336364_55 | Early | Intermediate | Bad |
| 15359858_55 | Early | Bad | Bad |
| 15359859_55 | Early | Bad | Bad |
| 15359860_55 | Early | Bad | Bad |
| 15387039_55 | Early | Intermediate | Bad |
| 15387043_55 | Late | Good | Good |
| 16174074_55 | Late | Intermediate | Bad |
| 16174075_55 | Early | Good | Good |
| 16252334_55 | Late | Good | Good |
| 16253684_55 | Early | Bad | Bad |
| 16253686_55 | Early | Bad | Bad |
| 16291658_55 | Early | Bad | Bad |
| 16291661_55 | Early | Bad | Bad |
| 16321374_55 | Early | Good | Good |

1.-59. (canceled)

60. A method of treating cancer in a patient having a cancer-related tumor, wherein the patient is likely to benefit from administration of TILs comparative to a group of other cancer patients that have been administered TILs, comprising the steps of:
obtaining from the patient a tumor fragment comprising a first population of TILs;
contacting the tumor fragment with a first cell culture medium;
performing an initial expansion of the first population of TILs in the first cell culture medium to obtain a second population of TILs, wherein the second population of TILs is at least 5-fold greater in number than the first population of TILs, and wherein the first cell culture medium comprises IL-2;
performing a rapid expansion of the second population of TILs in a second cell culture medium to obtain a third population of wherein the third population of TILs is at least 50-fold greater in number than the second population of TILs after 7 days from the start of the rapid expansion, wherein the second cell culture medium comprises IL-2, OKT-3 (anti-CD3 antibody), and irradiated allogeneic peripheral blood mononuclear cells (PBMCs), and wherein the rapid expansion is performed over a period of 14 days or less;
harvesting the third population of TILs; and
administering a therapeutically effective portion of the third population of TILs to the patient,
wherein the likelihood of beneficial administration of TILs to the patient is determined by a serum based analytical assay comprising:
obtaining an analytical signature of a blood-derived sample from the patient,
comparing the analytical signature with a training set of analytical signatures of samples from the group of other cancer patients that have been administers TILs, wherein the analytical signatures of the training set are class-labeled good, intermediate, bad, late, early, plus (+), or minus (−); and
classifying the analytical signature of the blood-derived sample from the patient with the class label good, late, or plus (+).

61. (canceled)

62. The method of claim 60, wherein subgroups of the other cancer patients that have been administered TILs achieved a complete response, a partial response, no response, a stable disease state, or a progressive disease state.

63. The method of claim 60, wherein subgroups of the other cancer patients that have been administered TILs had no disease progression for about one year, about two years, about three years, about four years, about five years, or more than five years.

64. The method of claim 60, wherein subgroups of the other cancer patients that have been administered TILs achieved progression free survival of less than 6 months, about 6 months, about 12 months, about 18 months, about 24 months, about 30 months, about 36 months, about 42 months, about 48 months, about 54 months, about 60 months, up to 60 months, or more than 60 months.

65. The method of claim 64, wherein the class label good, late, or plus (+) is associated with progression free survival of about 24 months, about 30 months, about 36 months, about 42 months, about 48 months, about 54 months, about 60 months, up to 60 months, or more than 60 months.

66. The method of claim 60, wherein the analytical signature of the blood-derived sample from the patient is obtained by a mass spectrometry method, an electrophoresis method, or a chromatography method.

67. The method of claim 60, wherein the analytical signature of the blood-derived sample from the patient is obtained by a mass spectrometry method, and the analytical signature comprises integrated intensity values of selected mass spectral features over predefined m/z ranges.

68. The method of claim 67, wherein the mass spectral features are correlated or anti-correlated with:
the complement system protein functional group, the acute inflammation protein functional group, the acute response protein functional group, or the acute phase protein functional group; or
the level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin.

69.-91. (canceled)

92. A method of treating cancer in a patient having a cancer-related tumor, wherein, compared to a healthy subject, the patient exhibits an increased or decreased level of expression of a protein selected from the group consisting of alpha1-Antitrypsin, C-reactive protein, fibrinogen gamma chain dimer, inter-alpha-trypsin inhibitor heavy chain H4, interleukin-27, tropomyosin beta chain, serum amyloid P, cyclin-dependent kinase 5:activator p35 complex, T-lymphocyte activation antigen CD80, mannose-binding protein C, alpha-S1-casein, calreticulin, haptoglobin, lymphatic vessel endothelial hyaluronic acid receptor 1, microtubule-associated protein tau, complement C1q, interleukin-6 receptor alpha chain, eukaryotic translation initiation factor 4A-III, integrin alpha-IIb: beta-3 complex, alpha2-antiplasmin, apolipoprotein E, C-reactive protein, complement C3b, complement C3b inactivated, complement C4b, complement C9, complement C3a anaphylatoxin, complement factor B, C1-esterase inhibitor, complement C1r, complement C3, serum amyloid P, complement C2, complement factor I, mitochondrial complement C1q subcomponent-binding protein, complement C5a, complement C8, complement C1s, complement C5b,6 complex, ATP-dependent DNA helicase II 70 kDa subunit, mannan-binding lectin serine peptidase 1, complement C6, P-selectin, ficolin-3, collagen alpha-1(VIII) chain, lipopolysaccharide-binding protein, D-dimer, serum amyloid A, and transferrin, the method comprising the steps of:
obtaining a tumor fragment comprising a first population of TILs;
contacting the tumor fragment with a first cell culture medium;
performing an initial expansion of the first population of TILs in the first cell culture medium to obtain a second population of TILs, wherein the second population of TILs is at least 5-fold greater in number than the first population of TILs, and wherein the first cell culture medium comprises IL-2;
performing a rapid expansion of the second population of TILs in a second cell culture medium to obtain a third population of wherein the third population of TILs is at least 50-fold greater in number than the second population of TILs after 7 days from the start of the rapid expansion, wherein the second cell culture medium comprises IL-2, OKT-3 (anti-CD3 antibody), and irradiated allogeneic peripheral blood mononuclear cells (PBMCs), and wherein the rapid expansion is performed over a period of 14 days or less;
harvesting the third population of TILs; and
administering a therapeutically effective portion of the third population of TILs to the patient.

93. The method of claim 92, wherein the cancer is selected from the group consisting of melanoma, ovarian cancer, cervical cancer, lung cancer, bladder cancer, breast cancer, head and neck cancer, renal cell carcinoma, acute myeloid leukemia, colorectal cancer, sarcoma, non-small cell lung cancer (NSCLC), estrogen receptor positive (ER⁺) breast cancer, progesterone receptor positive (PR⁺) breast cancer, human epidermal growth factor receptor 2 (HER2⁺) breast cancer, triple positive breast cancer (ER⁻/PR⁺/HER2⁺), triple negative breast cancer (ER⁻/PR⁻/HER2⁻), double-refractory melanoma, and uveal (ocular) melanoma.

94. (canceled)

95. The method of claim 92, wherein the level of expression of the protein is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

96.-101. (canceled)

102. The method of claim 92, wherein the initial expansion is performed over a period of 21 days or less.

103. The method of claim 102, wherein the initial expansion is performed over a period of 11 days or less.

104. The method of claim 92, wherein the rapid expansion is performed over a period of 7 days or less.

105. The method of claim 92, wherein the IL-2 is present at an initial concentration of between 1000 IU/mL and 6000 IU/mL in the first cell culture medium.

106. The method of claim 92, wherein the IL-2 is present at an initial concentration of between 1000 IU/mL and 6000 IU/mL and the OKT-3 antibody is present at an initial concentration of about 30 ng/mL in the second cell culture medium.

107.-110. (canceled)

111. The method of claim 92, further comprising the step of treating the patient with a non-myeloablative lymphodepletion regimen prior to administering the therapeutically effective portion of the third population of TILs to the patient.

112. The method of claim 111, wherein the non-myeloablative lymphodepletion regimen comprises the steps of administering cyclophosphamide to the patient at a dose of 60 mg/m²/day for two days followed by administering fludarabine to the patient at a dose of 25 mg/m²/day for five days.

113. The method of claim 92, further comprising the step of treating the patient with a high-dose IL-2 regimen starting on the day after administering the therapeutically effective portion of the third population of TILs to the patient.

114. The method of claim 113, wherein the high-dose IL-2 regimen further comprises aldesleukin, or a biosimilar or variant thereof.

115. The method of claim 114, wherein the aldesleukin, or the biosimilar or variant thereof, is administered at a dose of 600,000 or 720,000 IU/kg, as a 15-minute bolus intravenous infusion every eight hours until tolerance.

116. The method of claim 92, wherein the rapid expansion comprises co-culturing the second population of TILs with feeder cells.

117. The method of claim 92, wherein the IL-2 is present at an initial concentration of between 1000 IU/mL and 6000 IU/mL in the second cell culture medium.

118. The method of claim 92, wherein the the OKT-3 antibody is present at an initial concentration of about 30 ng/mL in the second cell culture medium.

119. The method of claim 92, wherein the first population of TILs comprises T cells with a phenotype selected from the group consisting of CD8⁺CD28⁺, CD8⁺CD27⁺, CD8⁺CD27⁺CD28⁺, CCR7⁺, and combinations thereof.


SEQUENCE LISTING (8 sequences)

SEQ ID NO: 1 - protein, 450 aa, Artificial Sequence
  Muromonab heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Muromonab light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, Homo sapiens
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, Artificial Sequence
  Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 130 aa, Homo sapiens
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 153 aa, Homo sapiens
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 7 - protein, 115 aa, Homo sapiens
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 132 aa, Homo sapiens
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS